(19) United States
(12) Patent Application Publication
(10) Pub. No.: US 2025/0213881 A1
(43) Pub. Date: Jul. 3, 2025

(54) METHODS TO MITIGATE SIDE EFFECTS OF CANCER TREATMENTS

(71) Applicant: All-Cells Healing Inc., New Castle, DE (US)

(72) Inventors: James Zhou Liu, New Castle, DE (US); Yuehua Gu, New Castle, DE (US)

(73) Assignee: All-Cells Healing Inc., New Castle, DE (US)

(21) Appl. No.: 19/009,885

(22) Filed: Jan. 3, 2025

Related U.S. Application Data
(63) Continuation-in-part of application No. 18/735,463, filed on Jun. 6, 2024.
Application No. 18/735,463 is a continuation-in-part of application No. 17/409,665, filed on Aug. 23, 2021.
(60) Provisional application No. 63/069,142, filed on Aug. 23, 2020.

(51) Int. Cl. A61N 5/06; A61K 45/06

(52) U.S. Cl. CPC A61N 5/06; A61K 45/06

(57) ABSTRACT

A bioenergy generator to increase the life force of a biological system is provided. The bioenergy generator includes fine pieces of natural stone, fine pieces of natural sand, fine pieces of metal, water and a supporting material operable to hold the natural sand, the natural stone, the metal, and the water together. Uses of the bioenergy generator are also provided, in particular, to enhance natural healing ability of a biological body and to treat a disease in a mammal, and to mitigate side effects of cancer treatments.

### CROSS-REFERENCES TO RELATED APPLICATIONS

This application is continuation-in-part of U.S. application Ser. No. 18/735,463, filed on Jun. 6, 2024, which is a continuation-in-part of U.S. application Ser. No. 17/409,665, filed on Aug. 23, 2021, which claims priority to U.S. Provisional Application No. 63/069,142, filed on Aug. 23, 2020, the disclosures of which are incorporated herein by reference in their entireties.

### BACKGROUND

### Field of the Invention

Maintaining the body free of any disease is a universal desire. The continuation of exploring the natural or artificial resource for reaching that healthy goal is the effort of mankind generation after generation. However, due to exposure to a variety of pathogenic factors, anybody would have aged cells/organs and get certain diseases during their lifetime. The questions were who would get which disorder/disease at which frequency. Before there is a way to have an endless lifespan, it is acceptable that the disease would be postponed as long as possible, or occur at the very late stage of life when the important cells of the body lost their cellular activities. The current invention is to provide a new bioenergy sphere generator to restore cellular activities, to slow down or even relatively reverse the aging process, and to treat diseases.

### Description of Related Art

About one hundred years ago, Nicola Telsa envisioned that “if you want to find the nature of the universe, think in terms of energy, frequency, and vibration.” Quantum phenomena were phenomenally baffling to many. First was wave-particle duality, in which light can act like particles, and particles such as electrons interfere like light waves. Second, a particle's position and momentum are hard-wired into physics. Third, we could have only probabilistic knowledge of a system: in Schrödinger's thought experiment, a cat in a box is both dead and alive until it is seen. Fourth, particles can become entangled. For example, two particles might have opposite spins, no matter how far apart they are: if you measure one to be spin up, you instantly know that the other is spin down. Einstein called this “spooky action at a distance”. The so-called spooky action has many health benefits revealed by many recent scientific studies.

Scalar waves (with spooky action) can strengthen human body health through energy. Preventing a disease from happening is the ideal medicine. Balanced nutrition is essential for maintaining normal health and body functions because nutrients are needed for every cell in the body. Facilitating nutrients absorption to the cell is one of the effective methods to main ideal nutrition. Vaccination is the most successful approach to prevent infectious disease by building an acquired immunity. A healthy lifestyle and positive attitude play very important roles for a long, healthy, and happy life. Chemoprevention with a modern drug is used to prevent or postpone certain diseases. When a person or an animal is sick, there is possibly more than one type of therapy to stop the disease process. Administration of a drug is the most popular treatment of a disease, and then, a surgery, as needed. However, if any therapy can directly increase the life force of a mammal to enhance innate healing, that therapy would be safer and much preferred. One of them was the energy medicine using subtle energy of scalar wave.

Subtle energy research helped advance the understanding of scalar waves. There are many different types of scalar waves. For example, there are vital scalar waves (corresponding with the vital life force or “Qi” body), emotional scalar waves, mental scalar waves, causal scalar waves, and so forth. In essence, as far as we are aware, all “subtle” energies are, made up of various types of scalar waves and biophoton. Scalar waves are conceived as longitudinal waves, as are sound waves. Unlike the transversal waves of electromagnetism, which move up and down perpendicularly to the direction of propagation, longitudinal waves vibrate in line with the direction of propagation. Transversal waves can be observed in water ripples: the ripples move up and down as the overall waves move outward, such that there are two actions; one moving up and down, and the other propagating in a specific direction outward. Longitudinal waves are waves to promote health. Some general properties of scalar electromagnetic waves (of the beneficial kind) include: travel faster than the speed of light; seem to transcend space and time; cause the molecular structure of water to become coherently reordered; positively increase immune function in mammals; are involved in the formation process in nature. Particularly, healthy scalar waves are beneficial to brain functions.

A healthy, high-functioning brain is essential to thrive in today's busy society. Keep a sharp mind by creating an environment that energetically supports top mental performance by improving focus, lifting mood, promoting overall brain health, and much more with an energetic boost.

Spiritual development is the right way for mental and overall health. One very important aspect of health is spiritual wellness. Subtle energy helps a wide range of applications that are designed to help heal, grow, and awaken spiritual and psychic abilities. This variety includes programs to open one's pineal gland, or the third eye, heal the heart center, and clear and protect the subtle energy fields from negative influences on life.

“What is life” and “How to have a healthy life” are very basic research questions for modern medicine. Science has focused on gathering all the right elements and on placing them under the right circumstances to initiate life. To a certain extent, elements and circumstances are important, but recent discoveries indicate that only when the elements become organized do they sustain the flow of energy and the flow of information necessary for the existence of life. This type of organization refers to a molecular organization like that found in crystals—repeating geometric patterns that can amplify and conduct signals. The molecular organization of crystals facilitates the flow of energy and information.

Many tissues and cellular components have now been identified as liquid crystals. They sustain the flow of energy and at the same time, the flow of energy sustains the liquid crystalline state. Quantum biologists and quantum physicists have begun to discover evidence such that life itself can be defined in terms of an organism's ability to maintain the liquid crystalline state. Lifeforce can be measured with a biometer.

The biometer is a scale that was developed by a French physician, Antoine Bovis, and the English Doctor Oscar Brunler, at the beginning of the last century. Initially classified utilizing commuting to unknown “vital rays”, this scale was intended to distinguish healthy and unhealthy foods and to enable better medical use. In the course of further research, it became clear that radiation is an emission of light quantum. Based on the “Bovis” scale were therefore the wavelengths of light. Due to the relativity of the intensity and vitality of the radiation, Bovis introduced the term “Bovis Unit.”

Dividing the “Bovis Units” by 10 gives the value for the corresponding wavelengths in nanometers (nm). In 1970, the Bovis scale was calibrated by a French engineer Siometon and is used ever since to determine general average values for various things in life. 6,300-6,800 BEH are considered for both a healthy diet and a healthy human. Lower values indicate a lack of energy; values above an increased vitality. A distinction as to whether drinks, food, living rooms, etc. do rather have an exhaustive or a stimulating effect. We need to know that life energy tends to achieve balance. If we take a bath in vital-poor water, the body energy tries to balance the energy of the water. That is why we do not feel refreshed after a bath in “dead” water, but rather sluggish and tired. Below were Bovis Values and meanings.
1,000—rotten or radioactively contaminated food, mold, asbestos
2,000—sweetened foods, saturated fats, chemical solvents
3,000—lead, aluminum, body care products with synthetic additives
4,000—fat cheese, fried potatoes, mercury, coke, preserved vegetables
5,000—iron, homogenized milk, tap water
6,000—cottage cheese, yogurt, cream
6,300-6,800—fresh vegetables, fresh milk, boiled potatoes, green lettuce, apple, pear, shitake mushrooms, wine, beer, fresh meat, brass, silver, wood, clay, cotton, wool.
7,000-8,000—rice, linseed oil, safflower oil, pineapple, leather
9,000—crystal rock, silk, lemon, Himalayan salt, spirulina algae
11,000—spring water, living water
12,000—religious building (Sacred Geometry)
28,000—Cheops pyramid (King's Chamber)
   Only an experienced person can measure Bovis life force units.

It is advantageous to have more than one energy source to treat certain disorders. Infrared (IR) treatment is a safe and effective therapy. Infrared radiation is electromagnetic radiation with wavelengths between 760 nm and 100,000 nm. Low-level light therapy (LLLT) or photobiomodulation (PBM) therapy generally employs light at red and near-infrared wavelengths (600-100 nm) to modulate biological activity. Many factors, conditions, and parameters influence the therapeutic effects of IR, including fluency, irradiance, treatment timing and repetition, pulsing, and wavelength. Increasing evidence suggests that IR can carry out photostimulation and photobiomodulation effects particularly benefiting neural stimulation, wound healing, and cancer treatment. Nerve cells respond particularly well to IR, which has been proposed for a range of neurostimulation and neuromodulation applications, and recent progress in neural stimulation and regeneration was reviewed by scholars. Far infrared rays penetrate the human body up to 6 inches deep into the muscles, tissues, joints, and more. Infrared heating pad emits far-infrared rays which can increase local blood circulation, remove metabolic wastes, and provide temporary relief of minor muscle and joint pain and stiffness, the relief of joint pain associated with arthritis, muscles spasms and strains, muscular back pain, muscle relaxation. A systematic review and meta-analysis of its effects and mechanism of action on pain relief and quality of life were performed by a group of investigators.

Infrared and scalar wave energy has safely been used for many years as medical devices. The applicant did a google search and determined that there were existing literature, standards, clinical and pre-clinical data that can adequately support the inventor's determination that there is no risk of using the new device.

Scalar wave energy has been safely used for more than 20 years in a variety of healing and diagnostic products. This was reviewed in The Barron Report that the NASA engineers were to develop the device of using scalar energy to care for the astronauts. A number of the health-beneficial products made by using scalar wave energy are in the consumer market, such as in www.amazon.com, like Scalar Wave Laser, or Tesla watches. Scalar wave energy is non-toxic, with no negative impact on the environment, no known side effects, non-invasive, and drug-free. The other published studies also support the use of scalar wave energy for promoting health, directly or indirectly. It is the right source for promoting health and wellness.

It is commonly known that better sleep can have many health benefits. When an efficient sleep was experienced nights after nights, many users gained positive health benefits like less stress, more energy, improved circulation, ease to breathe, reduced inflammation, faster recovery from heavy physical works or exercise, accelerated wound healing, body detoxification, normalizing frequency of urination, relief of muscle pain, headaches and muscle tension, reduced chronic pain, and a natural feeling of wellness, calm and balance in daily life, as reviewed by WebMD (WebMD: https://www.webmd.com/sleep-disorders/benefits-sleep-more#1) and NIH. (https://newsinhealth.nih.gov/2013/04/benefits-slumber).

Several scalar-wave-based devices have been used to treat a variety of diseases. The commonly used one type was the scalar wave laser. More than 50 types of diseases were successfully treated with scalar wave laser, such as asthma, allergy, sleep disorder, Alzheimer's disease, high blood pressure, stress, etc. Typing in “scalar wave laser” yielded 1.6 million entries in google search. However, performing any laser to achieve the actual therapeutic effect needs a therapist with a special skill. The example was that acupuncture needles can be used to treat a variety of diseases/disorders, even for anesthetizing the patient during surgery. However, using the needle requires former training either in school or by a long-term coach. If a patient can use a device for needleless acupuncture or self-healing, such as a bioenergy generator 100 provided in the current invention, that will be very welcome.

The other available scalar wave device is a scalar wave watch or Tesla watch. Some diseases and conditions were treated with scalar wave watches, such as stress. It is used as a wellness product made to combat the effects of electromagnetic hypersensitivity (EHS). Human bodies are disrupted by electromagnetic fields put out by everything from smartphones, microwave ovens, overhead electric cables, and even Wi-Fi. Those who suffer from it have a range of symptoms including headaches, poor sleep, hard to concentrate, and can reproduce flu-like symptoms. However, the Tesla watch can only provide a very low level of scalar wave energy. Treating certain important diseases would need a high level of scalar wave energy.

In the current market, a scalar wave device called Quantum Resonance Crystal Bed is used. It is a combination of crystals and light working together to heal the body. The transmitted energy cleanses, energizes, and balances the chakras or energy centers in the human body. It was reported that this Quantum Resonance Crystal Bed brings a deep sense of peace; reduces stress and anxiety, restores the body and boosts energy, balances the energy system inside the body, provides deeper, restive sleep, and more. Because the treatments are non-invasive and easy to perform, they are suitable for all ages. Those who benefit the most are people who are encountering stress, not feeling energetic or those who want to clear their minds and start anew. For those who try the crystal bed sessions complete with quantum resonance, the results have been quite noticeable. However, the Quantum Resonance Crystal Bed was not used as a typical bed. The seven crystal lights must focus on the 7 chakras to be effective.

Many scalar wave pendulums are sold in the market. Like a scalar wave watch, scalar wave pendulums can only provide a very low level of scalar wave energy. Its therapeutic effect is limited.

One of the devices used in Quantum Medicine is Quantum Resonance System (QRS). The QRS is to enhance cell preservation and cell regeneration. It can be applied to diseases and physical maladies. The QRS home system can be operated comfortably at home. It was reported that the QRS transports hydrogen to accomplish cellular entry of calcium, sodium, potassium, chlorine, and magnesium ions. Thus, it allows for better cell supply and freed the same cell of waste products and toxins. The QRS significantly increased oxygen in the tissues with the oxygen diffusion rate increased by 80 to 900%, and the energy molecules (ATP) were formed. This allows the QRS system to transport the materials through solidified or slackened tissue structures. The QRS, however, only provides stationary scalar energy to the human body. Thus, the body would not be uniformly energized from all directions. It would be more desirable that the bioenergy sphere is an important part to let the whole body receive the energy from all directions, not only from the blanket-like device the person laid on.

Spooky2 Scalar acts slightly differently from QRS. It has both a sender and a receiver of scalar-wave. The sender is tuned to the receiver so they are perfectly linked. There is only a single wire between the two. A scalar field is only produced when the two units are tuned. They become charge-coupled, effectively becoming one entity; one transmitting a regular electrical signal, and the other returning the signals in the form of a scalar field. The lids become a transmitting antenna of scalar energy, with a scalar field between them. The return path is the space between the lids. The scalar field goes in all directions, but a receiver will absorb this energy, in a similar way to the ionized gas in a plasma ball. The finger absorbs the energy, and a stream of electrons passes through the glass to the finger. When this happens, the energy becomes more focused. The person being treated sits between the sender and receiver, and they also become a tuned receiver. Spooky scalar is another device to broadcast scalar waves in a stationary manner, not in a circulatory manner. Therefore, the whole body would not be uniformly energized from all directions. Rather, the energy waves enter into the body from one side and leave the body from the other side.

It would be more desirable for the Spooky system to release the energy to form a sphere to let the whole body receive the energy from all directions, not only from one side go through the other side of the body.

Our disclosed invention was to provide a new composition and process to manufacture the bioenergy generator without using any electricity, as well as the uses thereof. The newly made products were test-used by many volunteers with different health conditions after each of them signed the informed consent form.

### SUMMARY OF THE PRESENT DISCLOSURE

In accordance with embodiments of the invention, a bioenergy generator to increase the life force of a biological system is provided. The bioenergy generator includes fine pieces of natural stone selected from the group of diamond, amethyst, tourmaline, jadestone with any color, and obsidian. The bioenergy generator further includes fine pieces of natural sand selected from the group of white sand, black sand, pink sand, red sand, yellow sand, orange sand, and green sand. The bioenergy generator further includes fine pieces of metal selected from the group of steel, iron, copper, zinc, aluminum, calcium, magnesium, silver, and gold. The bioenergy generator further includes water and a supporting material operable to hold the natural sand, the natural stone, the metal, and the water together.

The invention offers both stationary and portable bioenergy generators to produce life force (Qi, or Biophoton) and to meet a variety of purposes. The new bioenergy generators are powerful in producing life force without using any electricity, either plug-in or battery. The bioenergy field is formed by the presence of the bioenergy generator. The bioenergy field is around the entire body and increases the cellular bioenergy, to increase cellular repair rate, to decrease cell destruction rate, to promote the cell metabolism, to detoxify toxins or free radicals, to remove waste products from the cells, and to bio-recharge cells to the full voltage level for maintaining optimum health of a mammal. All cells and all organs in the whole body become energized. For a biosystem, such as a wine fermentation tank, the bio-reaction speed became much faster than before. The biotech company can utilize the newly created bioenergy generator and related biophoton technology to speed up the production of pharmaceutical materials. The hospital and clinic rooms can be equipped with the newly created non-electricity-involved bioenergy generator to benefit these patients and medical professionals at the same time. The special plants can be grown in the bioenergy field powered with the newly created bioenergy generator. The application of this new technology is extremely broad.

An objective of the present invention is to provide a serial of bioenergy generators, and a combinational-generator to produce biophoton, scalar wave, infrared wave, and negative ion, to help a biological system to be energized and/or to improve the functionality. Then, the health and body function of a mammal can be brought back to an optimum status. The invention incorporates the bioenergy and far infrared energy into one device that can broadcast a bio-healing energy field in a stationary and/or portable format, which can facilitate the energy recharge of the cells in any part of the body. Utilizing the bioenergy generator, the inventors have helped many patients to improve their health conditions significantly and treated many important diseases, such as depression, attention-deficit-hyperactivity disorder (ADHD), paralysis after stroke, terminal cancers, Alzheimer's disease, dementia, hypertension, diabetes, traumatic brain injury, Parkinson's disease, etc. These people with chronic fatigue were recharged and their daily lives were full of energy. Some patients with insomnia changed to have a long-time waited for normal sleep. The depressed patients became normal individuals and were happy. The patients with mental stress were relieved. The elderly patients improved their life quality due to free of life without any physical restriction. Besides, the bottled wine was enhanced in the life force, in taste and flavor by the presence of the newly invented bioenergy generator. Domestic pets became healthier and could move freely, and even could run again after suffering arthritis for a long time when the invented bioenergy generator was placed in the sleeping space. The flowers had a long blossom time after the bioenergy generator was placed nearby. The in-house trees were healthier than before the placement of the invented bioenergy generator.

One general aspect of the present disclosure relates to a method of generating biophotons to enhance natural healing ability of a biological body, wherein the method comprises:
a) selecting and using a mixture of active stones comprising at least two types of stones with distinct electromagnetic properties, such as amethyst, tourmaline, jadestone, obsidian, and diamond;
b) selecting and using fine pieces of natural sand selected from the group consisting of white sand, black sand, pink sand, red sand, yellow sand, orange sand, and green sand;
c) selecting and using fine pieces of a metal, such as iron, copper, zinc, aluminum, calcium, magnesium, silver, and gold;
d) selecting and using water, such as natural water or energized water;
e) selecting and using a supporting material, such as mortar, to hold said natural stones, said natural sand, said metal, and said water together to form a stable solid mixture;
f) arranging said solid mixture in proximity to the biological body;
g) letting said biological body capture biophotons released from said solid mixture; and
h) allowing said biological body to utilize said biophotons to get the benefits of said biophotons.

Another general aspect of the present disclosure relates to a method of generating biophotons to treat a disease in a mammal, the method comprises:
a) selecting and using a mixture of active stones comprising at least two types of stones with distinct electromagnetic properties, such as amethyst, tourmaline, jadestone, obsidian, and diamond;
b) selecting and using fine pieces of natural sand selected from the group consisting of white sand, black sand, pink sand, red sand, yellow sand, orange sand, and green sand;
c) selecting and using fine pieces of a metal, such as iron, copper, zinc, aluminum, calcium, magnesium, silver, and gold;
d) selecting and using water, such as natural water or energized water;
e) selecting and using a supporting material, such as mortar, to hold said natural stones, said natural sand, said metal, and said water together to form a stable solid mixture;
f) arranging said solid mixture in proximity to the mammal;
g) letting said mammal capture biophotons released from said solid mixture; and
h) allowing said biophotons treat the disease.

In some embodiments, the disease is chronic obstructive pulmonary disease (COPD), asthma, hypertension, diabetes, post-COVID, lack or decrease of libido, aging, pain, cancer, side effects of cancer chemo or radiotherapy, uncontrolled glaucoma, Alzheimer's disease, dementia, myasthenia gravis, arthritis, or congestive heart disease.

In some embodiments, said mammal is a human such as a patient, or a domesticated animal.

Other features and advantages of the present disclosure are apparent from additional descriptions provided herein, including different examples. The provided examples illustrate different components and methodology useful in practicing the present disclosure. Such examples do not limit the claimed disclosure. Based on the present disclosure, the skilled artisan can identify and employ other components and methodology useful for practicing the present disclosure.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1 depicts a person sleeping inside a three-dimensional bioenergy field filled with bioenergy particles.

FIG. 2 depicts a three-dimensional distribution of the energy particles from a bioenergy field.

FIG. 3 depicts a diagram of a sphere bioenergy generator with a radius of 1 meter in accordance with embodiments of the invention.

FIG. 4. depicts an image of a rectangular bioenergy generator in accordance with embodiments of the invention.

FIG. 5 depicts an image of a cylindrical bioenergy generator in accordance with embodiments of the invention.

FIG. 6. depicts an image of an oval bioenergy generator in accordance with embodiments of the invention.

FIG. 7 depicts an image of a cubic bioenergy generator in accordance with embodiments of the invention.

FIG. 8 depicts an image of a ball-shaped bioenergy generator in accordance with embodiments of the invention.

FIG. 9 depicts an image of a rod-like bioenergy generator in accordance with embodiments of the invention.

FIG. 10 depicts an image of a bioenergy generator in a rectangular shape with inner space in accordance with embodiments of the invention.

FIG. 11 depicts an image of the contents inside of a bioenergy generator in a rectangular shape in accordance with embodiments of the invention.

FIG. 12 depicts an image of the contents inside of a bioenergy generator in a square shape in accordance with embodiments of the invention.

FIG. 13 depicts an image of the content inside of a bioenergy generator in a cylinder shape in accordance with embodiments of the invention.

FIG. 14 depicts an illustration of a bed energized with multiple bioenergy generators in accordance with embodiments of the invention.

FIG. 15 depicts an illustration of a table energized with multiple bioenergy generators in accordance with embodiments of the invention.

FIG. 16 depicts an illustration of a chair energized with multiple bioenergy generators in accordance with embodiments of the invention.

FIG. 17 depicts an illustration of a backpack energized with a bioenergy generator in accordance with embodiments of the invention.

FIG. 18 depicts an image of bioenergy scan produced by using a Bioplasm 9D-NLS Bioresonance Machine (9D-NLSBM) to show the improved cellular activities in the brain after a male patient was treated with the bioenergy generator in accordance with embodiments of the invention.

FIG. 19 depicts an image of a bioenergy scan using a 9D-NLSBM showing the improved cellular activities in the neck veins after a male patient was treated with the bioenergy generator in accordance with embodiments of the invention.

FIG. 20 depicts an image of a bioenergy scan using a 9D-NLSBM showing the improved cellular activities in the retina after a male patient was treated with the bioenergy generator in accordance with embodiments of the invention.

FIG. 21 depicts an image of a bioenergy scan using 9D-NLSBM showing the improved cellular activities of the major organs after a male patient was treated with the bioenergy generator in accordance with embodiments of the invention.

FIG. 22 depicts an image of a bioenergy scan using 9D-NLSBM showing the improved cellular activities in the pancreas and spleen after a male patient was treated with the bioenergy generator in accordance with embodiments of the invention.

FIG. 23 depicts an image of a bioenergy scan using 9D-NLSBM showing the improved cellular activities in the kidney after the man was treated with the bioenergy generator in accordance with embodiments of the invention.

FIG. 24 depicts an image of a bioenergy scan using 9D-NLSBM showing the improved cellular activities in the intestine after a male patient was treated with the bioenergy generator in accordance with embodiments of the invention.

FIG. 25 depicts a bioenergy generator placed next to the fermentation tank to increase alcohol content in accordance with embodiments of the invention.

FIG. 26 depicts an alcohol fermentation tank bubbling after the bioenergy generator was placed on the fermentation tank in accordance with embodiments of the invention.

FIG. 27 depicts a measurement of increased alcohol content resulting from placing a bioenergy generator in accordance with embodiments of the invention next to a fermentation tank compared to a control fermentation tank.

FIG. 28 depicts a pad filled with bioenergy generating materials in accordance with embodiments of the invention.

FIG. 29 depicts a bioenergy generator in a ready-to-sell status in accordance with embodiments of the invention.

FIG. 30 depicts a blood sample examined under a microscope showing blood fluidity.

FIG. 31 depicts blood sample examined under a microscope showing a crystal water in the blood.

FIG. 32 depicts a bar graph comparison of the group SF-36 score of a cancer patient before and after using bioenergy generators in accordance with embodiments of the invention.

FIG. 33 depicts a bar graph comparison of the group SF-36 score of a dementia patient before and after using bioenergy generators in accordance with embodiments of the invention.

FIG. 34 depicts a bar graph comparison of the group SF-36 score of a patient with seizure before and after using bioenergy generators in accordance with embodiments of the invention.

FIG. 35 depicts a bar graph comparison of the group SF-36 score of a patient with stroke before and after using bioenergy generators in accordance with embodiments of the invention.

FIG. 36 depicts an bioenergy comparison of the patient with back pain before and after using bioenergy generators in accordance with embodiments of the invention.

FIG. 37 depicts a meridian bioenergy comparison of the volunteer before and after using bioenergy generators in accordance with embodiments of the invention.

FIG. 38 depicts the relationship between the pain reduction efficacy and the original pain levels in Example 93.

FIG. 39 depicts the improvement of Functional Status Scale (PCFS, a 5-grade version) during the 2-week study period in Example 94.

FIG. 40 depicts the decrease of severity of Post-COVID-19 symptoms during the 2-week study period in Example 94.

FIG. 41 depicts the spirometry tests of Post-COVID-19 patients in Example 94.

FIG. 42 depicts the changes of spirometry test of Post-COVID-19 patients during the 2-week study period in Example 94.

FIG. 43 depicts the life quality SF-36 score changes from baseline in Example 95.

FIG. 44 depicts the clinical study outcomes using Montreal cognitive assessment (MOCA) to measure the patient's cognitive capacity in Example 95.

FIG. 45 depicts the clinical study outcomes using Montreal cognitive assessment (MOCA) to measure the patient's cognitive capacity in Example 96.

FIG. 46 demonstrates the changes of the indicators of the patient's common laboratory tests during cancer treatment in Example 98.

FIG. 47 demonstrate the improvement of life quality during the 4-week study period in Example 100.

### DETAILED DESCRIPTION

The embodiments of the present disclosure will be best understood by reference to the drawings, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the system and method of the disclosure, as represented in the Figures is not intended to limit the scope of the disclosure, as claimed, but is merely representative of possible embodiments of the disclosure.

In accordance with embodiments of the invention, a bioenergy generator 100 to increase the life force of a biological system is provided. The biological system may be a mammal, a plant, a biological body, or a fermentation tank, for example.

The bioenergy generator 100 includes fine pieces of natural stone 102 selected from the group of diamond, amethyst, tourmaline, jadestone with any color, and obsidian. The bioenergy generator 100 further includes fine pieces of natural sand 104 selected from the group of white sand, black sand, pink sand, red sand, yellow sand, orange sand, and green sand. The bioenergy generator 100 further includes fine pieces of metal 106 selected from the group of steel, iron, copper, zinc, aluminum, calcium, magnesium, silver, and gold. The bioenergy generator further includes water 108 and a supporting material 110 operable to hold the natural sand, the natural stone, the metal, and the water together as a mixture 111. The supporting material may be a polymer, grout, water, sand, stone, or adhesion material, for example. A container 112 is operable to hold the natural stone 102, the natural sand 104, the metal 106, the water 108, and the supporting material 110. The increase in life force is measured by an increase in Bovis values of the biological system, a Bioplasm 9D-NLS Bioresonance Machine, a Biophoton detector, Gas discharge visualization (GDV), an increase in compensatory reactions, or an increase in a proof of an alcohol fermented in the fermentation tank.

The bioenergy generator 100 disclosed in the present invention includes at least one kind of the active stone with a minimum level of life force that has a certain healing effect on a mammal, such as a diamond; one kind of sand with a minimum level of life force, one kind of fine metal wire with a minimum life force, and a supporting system. Each ingredient can be further activated by using a strong biophoton generator. The bioenergy generator 100 can automatically and constantly generate and release the life force to a 3 dimensional field in which a mammal can rest, sleep, heal, and be energized.

In accordance with embodiments of the invention, a process to manufacture the bioenergy generator 100 is disclosed. In a first step, ingredients are selected, which in an embodiment include: natural stones 102 in fine pieces with a measurable life force energy by using the Bovis scaler; natural sands 104 in fine pieces with a measurable life force energy by using the Bovis scaler; safe metal 106 in fine pieces with a measurable life force energy by using the Bovis scaler; water 108 with a measurable life force energy by using the Bovis scaler; and an unactivated ingredient 110, such as polymer or grout, with or without life force.

In a second step, the ingredients are activated by placing the natural stones, natural sands, safe metal, and water next to a strong biophoton generator. The second step may be performed for at least 7 days, for example.

In a third step, the activated ingredients are weighed to achieve an exemplary ratio of ingredients natural stones 102, natural sands 104, safe metal 106, and water 108 in the ratio: 10%:10%:10%:10%, and unactivated ingredient 110 at 60%. If the net weight of the content of the bioenergy generator 100 is 1000 g (1 kg), then said ingredients natural stones 102, natural sands 104, safe metal 106, and water 108 will be 100 g, 100 g, 100 g, 100 g and unactivated ingredient 110 600 g. The ratio can be varied in a range that those said activated ingredients natural stones 102, natural sands 104, safe metal 106, and water 108 can be in the ratio of 1%:1%:1%:1% and unactivated ingredient 110 at 96%. The ratio can be varied in a range that those said activated ingredients natural stones 102, natural sands 104, safe metal 106, and water 108 can be in the ratio of 1%:1%:1%:1% and unactivated ingredient 110 at 96%, to 20%:20%:20%:20% and unactivated ingredient 110 at 20%.

In a fourth step, mix the weighed activated ingredients natural stones 102, natural sands 104, safe metal 106, and water 108, and unactivated ingredient 110 by adding unactivated ingredient 110 first to the mixer; then add the weighed activated ingredients natural stones 102, natural sands 104, safe metal 106, and water 108.

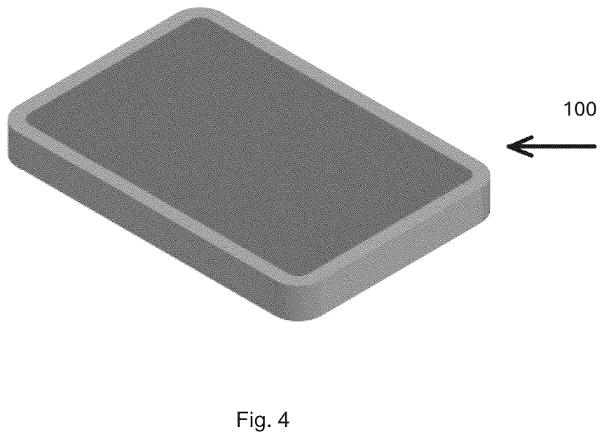

In a fifth step, pack the mixture into a container 112, such as a stainless steel container or sate plastic container. A variety of shapes of shapes may be used, as illustrated in FIGS. 4-13. The mix produced during the fourth step is added into the container. The net weight of the mix can be varied from 10 grams to 100 kg, for example. The common weight is from 1 kg to 20 kg. However, weights outside of this ranges are useful in some embodiments.

In a sixth step, measure bioenergy level using (1) Bovis scaler; (2) a group of panelists who were screened to be very sensitive to life force energy; (3) Lifeforce measurement instrument, such as a biophoton detector.

In one embodiment, the ratio of the mixture of ratio of ingredients natural stones 102, natural sands 104, safe metal 106, and water 108 was 15%:15%:15%:15%, and unactivated ingredient 110 at 40%. The net weight of the content of the bioenergy generator was 1000 g (1 kg). The user could use 1 to 4 of the small bioenergy generators 100 to aid in treating these conditions: leg skin temperature changes, finger oxygen saturation, back pain, heart disease, diabetes, high blood pressure, calm/falling asleep, chronic fatigue syndrome, insomnia, gynecological dysmenorrhea, prostate enlargement, depression, viral infection, male function, mental health, seizure, and dementia.

In one embodiment, the ratio of the mixture of ratio of ingredients natural stones 102, natural sands 104, safe metal 106, and water 108 was 15%:15%:15%:15%, and unactivated ingredient 110 at 40%. The net weight of the content of the bioenergy generator is 12 kg. The user could use 1 to 6 of the big bioenergy generators 100 to aid in treating these conditions: stroke, terminal cancer, untreatable cancer, stroke-paralysis, improving cellular activities of cerebral arteries, improving cellular activities of neck vein, improving cellular activities of retina, improving cellular activities of major organs, improving cellular activities of pancreas and spleen, improving cellular activities of kidney, improving cellular activities of intestine, and speed up fermentation process.

In one embodiment, the bioenergy generator 100 is operable to promote blood circulation. The leg-skin temperature and finger oxygen saturation were measured before and after the 15-minute use of the bioenergy generator 100. The test results are shown in Tables 1(a) and 1(b). The test results indicate that the device could promote blood circulation to warm the body surface and finger circulation reflected by increased oxygen saturation level.

TABLE 1(a)
Leg Skin Temperature Changes in Three Panelists

| | | 15-Minute | 15-Minute |
|---|---|---|---|
| | | After Use | After Use |
| | Before Use | (Treatment) | (Placebo) |
| Panelist 1 | 36.8 | 37.4 | 36.7 |
| Panelist 2 | 36.9 | 37.5 | 36.9 |
| Panelist 3 | 36.7 | 37.3 | 36.8 |
| Average | 38.8 +/− 0.1 | 37.4 +/1 0.1 | 36.8 +/− 0.1 |

TABLE 1(b)
Finger Oxygen Saturation in Three Panelists

| | | 15-Minute | 15-Minute |
|---|---|---|---|
| | | After Use | After Use |
| | Before Use | (Treatment) | (Placebo) |
| Panelist 1 | 96 | 97 | 96 |
| Panelist 2 | 97 | 98 | 97 |
| Panelist 3 | 95 | 96 | 95 |
| Average | 96 | 97 | 96 |

In one embodiment, the bioenergy generator 100 is operable to promote pain relief. A group of volunteers with bodily pains were to test if the bioenergy generator 100 had any impact on their bodily pains. All were free-living in their homes. They placed the Bioenergy Generator 100 (true or not-true) labeled with codes on their nightstands during the overnight sleeping. Each test-use was for 3 nights with each coded device. The bodily pains were reported at the end of the three-days test use period. The test results were shown in Table 2.

TABLE 2
Pain Relief for Five Volunteers

| | | 3 Nights |
|---|---|---|
| | Before Use | After Use |
| Patient 1, Female, | Pain level at 8 | Pain level at 2 |
| Back Pain | | |
| Patient 2, Male, | Pain level at 7 | Pain level at 1 |
| Shoulder Pain | | |
| Patient 3, Male, | Pain level at 7 | Pain level at 2 |
| Back Pain | | |
| Patient 4, Female, | Pain level at 6 | Pain level at 1 |
| Back Pain | | |
| Patient 5, Female, | Pain level at 6 | Pain level at 1 |
| Finger Pain | | |

These test results indicated in Table 2 show that the device can be used to reduce bodily pains.

In one embodiment, the bioenergy generator 100 is operable to aid in therapeutic effects. Through the actual use-testing among 310 users, it was observed that the bioenergy generator 100 has the obvious therapeutic effects on major diseases, such as stroke, heart disease, diabetes, high blood pressure, terminal cancer, and many other health conditions. The elderly who were physically weak and unable to take care of themselves can have a free-living life after lying down on the bed empowered with bioenergy generators 100 for a week. Besides, the bioenergy-generator treatment can rapidly improve the health status for those with sub-optimum health. No adverse events occurred during the use of the device, which was in agreement with all published studies showing that scalar wave energy did not have any side effects.

As observed, soon after the users entered into the bioenergy generator 100 powered bed and lie down, most users became calm and wanted to sleep, and it was easy for them to get into a deep sleep to heal their health conditions naturally and easily.

After a short-term treatment inside of the bioenergy field created by a bioenergy generator 100, the patient with depression, and even did not want to live in the past could open their mind and be happy to have a new life. The depressed person eliminated their boredom after being treated with the bioenergy generator 100.

The bioenergy generator 100 100 is operable to aid in the treatment of many diseases and conditions. Examples of these applications are below.

In one embodiment, the bioenergy generator 100 is operable to aid in the treatment of chronic fatigue syndrome. Thirty patients suffered from chronic fatigue syndrome. They had physical and mental exhaustion, some with the difficulty of walking. After treated with bioenergy generator 100 for at least two nights or times, all 30 patients had significant recovery in physical strength, and those symptoms of the fatigue were eliminated. The mood became pleasant; the memory was improved; and they felt 5 to 10 years younger than their current age for these who slept inside of the bed powered with bioenergy generators 100 for longer than 1 month.

In one embodiment, the bioenergy generator 100 is operable to aid in the treatment of insomnia. The 10 insomniac individuals were treated with the bioenergy generator 100. The 7 patients significantly improved their sleep, and it was easy to fall asleep, and the deep sleep time was prolonged. Among them, they had insomnia for more than 10 years, some even decades. Patients who failed by multiple treatments with modern medicines were treated with bioenergy generator 100 for two to three weeks, and their quality of sleep was significantly improved, so they could have a deep sleep.

In one embodiment, the bioenergy generator 100 is operable to aid in the treatment of back pain. The 52 middle-aged and elderly people with back pain were treated with the bioenergy generator 100, and 45 people significantly improved their quality of life, and almost eliminated their back pain. Among them, the 35 elderly regained their physical strength and could live pain-free. The 10 elderly people did not need their walking sticks after the treatment.

In one embodiment, the bioenergy generator 100 is operable to aid in the treatment of high blood pressure. The 30 patients with high blood pressure did not need antihypertensive drugs after using the bioenergy generator 100 for therapy at least for one week.

In one embodiment, the bioenergy generator 100 is operable to aid in the treatment of diabetes. The 19 people with diabetes did not rely on insulin injection after using the bioenergy generator 100 therapy at least for one week.

In one embodiment, the bioenergy generator 100 is operable to aid in the treatment of untreatable cancer. Sixteen cancer patients were treated with the bioenergy generator 100. Five advanced lung cancer patients prolonged their lifespan, and three advanced colon cancer patients were out of the dangerous phase and entered the rehabilitation period. One leukemia patient was back to the normal life. Three patients with prostate cancer were free from their bone pain. One patient with prostate cancer gained energy during the radiotherapy period. Three breast cancer patients gained energy and improved life quality significantly.

In one embodiment, the bioenergy generator 100 is operable to aid in the treatment of gynecological dysmenorrhea. The 30 women had an uncomfortable feeling, particularly backache during the period before treatment. After treated for two or three nights inside of the bed powered with bioenergy generators 100, 28 women had no more painful menstruation. Four women had a recurrence of the period after menopause for a year or so.

In one embodiment, the bioenergy generator 100 is operable to aid in the treatment of prostate enlargement. The 8 male patients with prostate enlargement had a high frequency of night urination. After treated with bioenergy generator 100 for two to five nights, their conditions improved significantly. The 7 people reduced the number of night urination to 1 time.

In one embodiment, the bioenergy generator 100 is operable to aid in the treatment of depression. Thirty people had depression. They were irritated, some even did not want to live. After sleeping for two or three months inside of the bed powered with bioenergy generator 100, 20 people were happy and cheerful, and their attitude towards life was positive.

In one embodiment, the bioenergy generator 100 is operable to aid in the treatment of a viral infection. bioenergy generator 100 was used to help 8 flu-patients to recover quickly from the viral infection by at least 3 days and reduced or eliminated the body pain caused by the influenza virus.

In one embodiment, the bioenergy generator 100 is operable to aid in the treatment of erectile dysfunction. The 36 male patients had a certain degree of erectile dysfunction before the treatment. After treated for 3 or more nights, a supermajority of these men (29) improved their libido which was reflected by the reappearance of the morning erection phenomenon.

In one embodiment, the bioenergy generator 100 is operable to aid in the treatment of mental health. As experienced by the majority of the 10 users, the bed powered with bioenergy generators 100 increased the consciousness, improved emotions of the patients, and helped them to quickly enter the state of calm and peace from the arrogant disorder. It helped to achieve a harmonious, pleasant, and balanced high-frequency state of the body, mind, and spirit. It also increased intuition and enhance concentration.

In one embodiment, the bioenergy generator 100 is operable to aid in the treatment of stroke-paralysis. Fourteen paralyzed patients due to stroke were treated. The fastest time for a patient to regain partial mobility was 6 days-nights of lying on the bed powered with 8 bioenergy generators 100. The average period to regain freedom of living was 40 days. One of them was a 49 years old African-American lady. After being treated for 6 days-nights with the bioenergy generators 100, she could walk easily without using a walker. Her body function recovered completely after one week of using bioenergy generators 100.

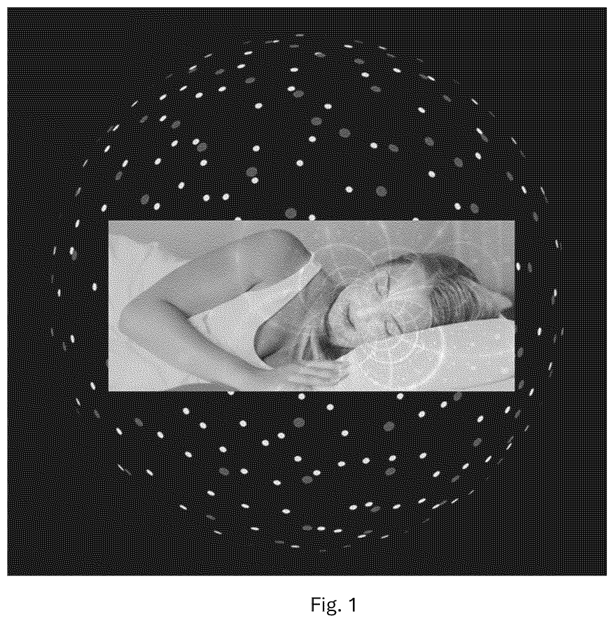

In the embodiment shown in FIG. 1, the bioenergy field 114 is created by a bioenergy generator 100 can recharge the body with bioenergy when the person is sleeping, awakening, or working. The bioenergy particles, like photons, carry bioenergy without mass. The bioenergy particles are three-dimensional, not a line or a point. They are tiny spinning waves. These spinning waves can penetrate the body easily. This is particularly advantageous in treating brain disorders since pharmaceutical particles are hard to be carried into the brain due to the blood-brain barrier (BBB).

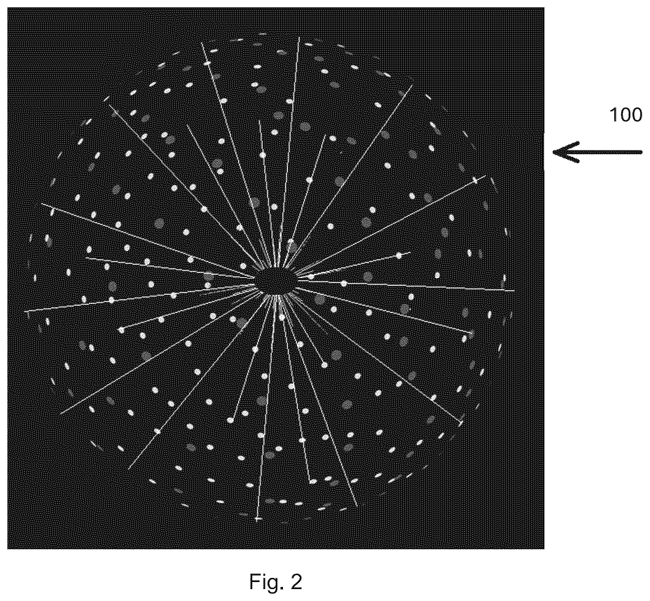

In the embodiment shown in FIG. 2, the bioenergy sphere 116 is created by a bioenergy generator 100 (in the center as a solid ball) is filled with bioenergy particles 118. The bioenergy particles 118 were concentrated nearby the bioenergy generator 100. The concentration becomes less and less as the radius of the sphere becomes longer and longer from the device 100, as measured for the strength of the bioenergy.

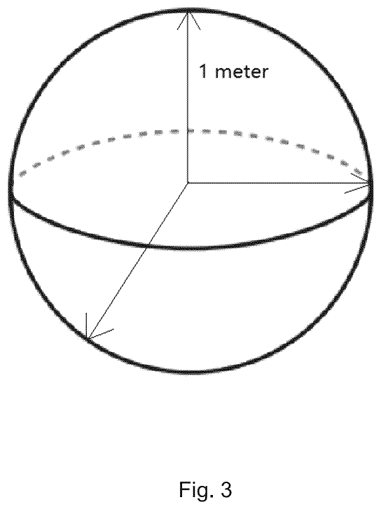

In the embodiment shown in FIG. 3, the bioenergy sphere 116 has a radius. The radius can vary from a few centimeters to several meters. For treating a mammal, the radius can be 1 to 2 meters since the supermajority of the people can be within the bioenergy sphere with a radius of 1 to 2 meters as measured.

In the embodiment shown in FIG. 4, the bioenergy generator 100 is in a rectangular shape. The short side of the generator can vary from a few centimeters to several meters. When the bioenergy generator 100 was made in a large size, such as longer than 1 meter for the short side and 2 meters for the long side with 0.5 meters in depth, the bioenergy field can be large enough to empower a whole house with 2200 square feet according to the bioenergy measurement.

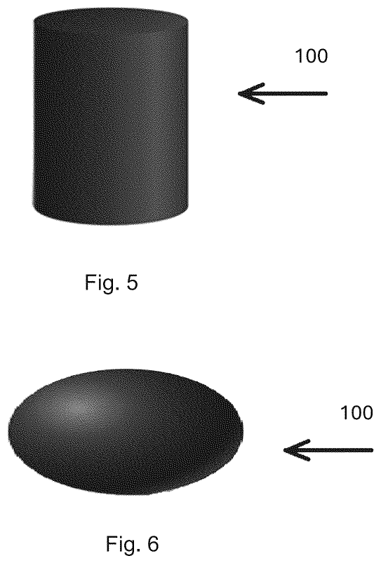

In the embodiment shown in FIG. 5, the bioenergy generator 100 is in a cylinder shape. The radius of the cylinder can vary from a few centimeters to several meters. When the bioenergy generator 100 was made in the large size, such as 1 meter in diameter and 2 meters in height, the bioenergy would be enough to empower a swimming pool with a 20 meters×30 meters with a depth of 1 meter of water according to the bioenergy measurement.

In the embodiment shown in FIG. 6, the bioenergy generator 100 was made in an oval shape. The length of the short and long diameter can vary from a few centimeters to several meters. For helping a woman to gain the ovary function, the size of the bioenergy generator 100 can be made with the short diameter as 10 centimeters, and long diameter, 15 centimeters. The bioenergy released from the device 100 can enter into the user's body to increase a user's ovary energy level according to the measurement before and after 1 hour of use.

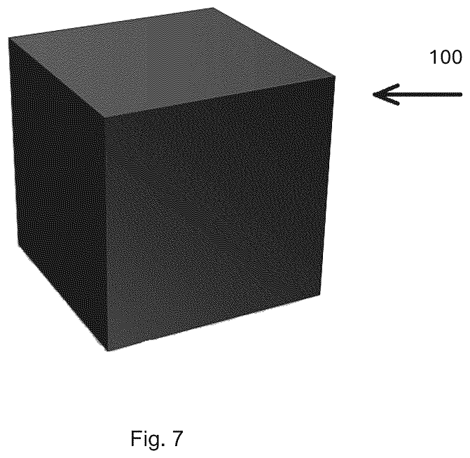

In the embodiment shown in FIG. 7, the bioenergy generator 100 was made in a cubic shape. The side of the cube can vary from a few centimeters to several meters. For empowering a clinic room with 14×14 feet size, the side of the cube was 20 to 40 centimeters.

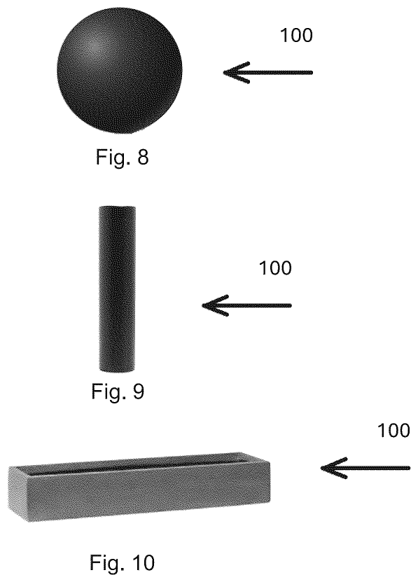

In the embodiment shown in FIG. 8, the bioenergy generator 100 was made in a ball shape. The diameter of the ball can vary from a few centimeters to several meters. For promoting the growth of farm animals, such as chickens, ducks, pigs, sheep, cows, horses, the size of the bioenergy generator 100 can be as large as the animal or as small as 1% of the animal in size or weight. If for promoting the health of domestic pets, such as dogs or cats, the size of the bioenergy generator 100 can be as large as the pet, or as small as 1% of the pet in size or weight. If for promoting a fermentation process, like making wines, the size of the bioenergy generator 100 can be as large as a 10% of the fermentation tank, or as small as 1% of the fermentation tank. The bioenergy released from the device can enter into the body of animals or the fermentation tank. As tested, the common domestic animals, like dogs or cats, can be energized when a bioenergy generator 100 was placed next to them than the control.

In the embodiment illustrated in FIG. 9, the bioenergy generator 100 was made in a long rod shape. The length of the rod can vary from a few centimeters to several meters, and the diameter can be a few centimeters to 1 meter. For helping a senior to sleep better, the size of the bioenergy generator 100 was made with the diameter of 8 centimeters and the length of 30 centimeters, then it was placed alongside of the user's pillow. The bioenergy released from the device 100 was effective to promote the user's sleep according to the feedback from the user's immediate family member.

In the embodiment shown in FIG. 10, the bioenergy generator 100 was made in a long rectangular shape and with a space inside of the rectangular. The length of the rectangle can vary from a few centimeters to several meters, and the other two sides can be a few centimeters to 1 meter. For helping to bioenergize a meat product, the rectangle bioenergy generator 100 was made with the length of 2 meters and the other two sides, 1 meter. The internal space is 1.6×0.8×0.8 diameters. The bioenergy released from the device 100 was effective to bioenergize the meat from 14K to 21K life force units within 48 hours according to the measurement.

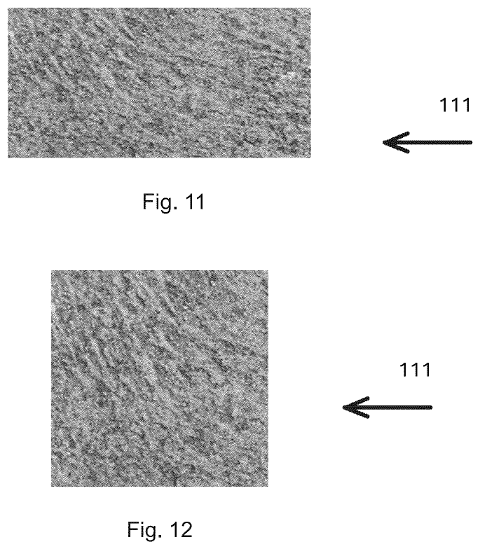

In the embodiment illustrated in FIG. 11, the content 120 inside of the package of a bioenergy generator 100 can be made in a rectangular shape. The size of the rectangle can vary from 0.1 cubic meters to 2 cubic meters.

In the embodiment illustrated in FIG. 12, the content 120 inside of the package of a bioenergy generator 100 can be made in a cubic shape. It can also be made like a ball. The size of the cube or a ball can vary from 0.1 cubic meters to 2 cubic meters.

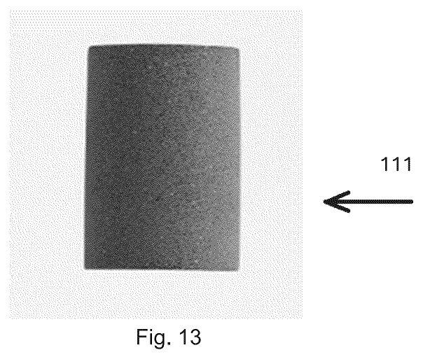

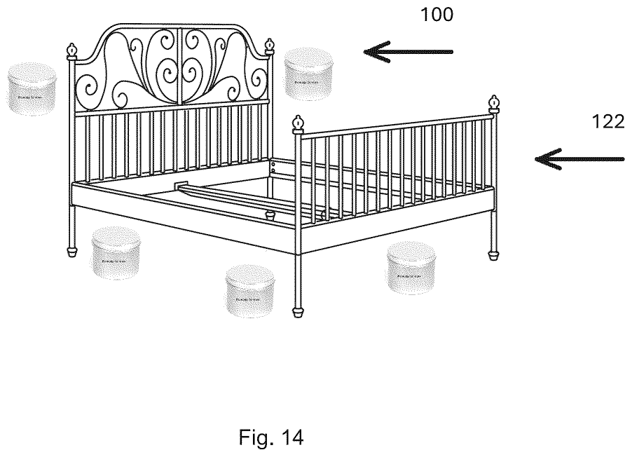

In the embodiment illustrated in FIG. 13, the content 120 inside of the package of a bioenergy generator 100 can be made in a cylinder shape. It can also be made like a rod. The size of the cylinder or a rod can vary from 0.1 cubic meters to 2 cubic meters.

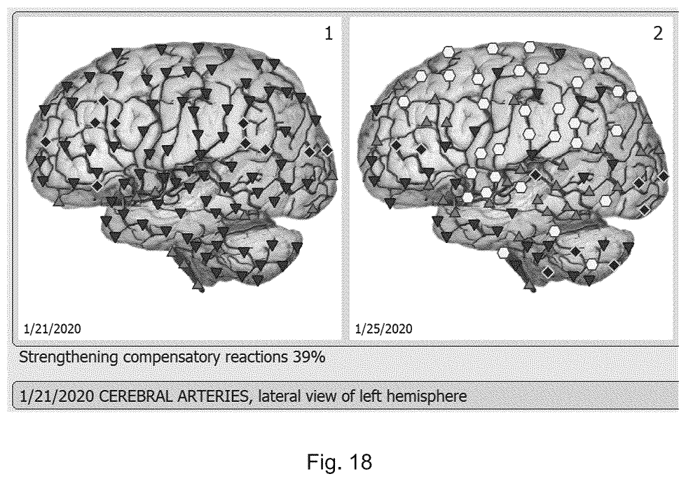

In the embodiment illustrated in FIG. 14, the bioenergy generator 100 can be placed anywhere in a relationship with a bed 122. The number and bioenergy strength of the bioenergy generator 100 can be varied depending on the user's need for the bioenergy, such as the different age or health condition. Common people using a bioenergy-empowered bed to gain energy would need one or two bioenergy generators 100 for promoting wellbeing and working efficiency. For these aged patients with chronic disease, such as arthritis, would need 4 or more bioenergy generators 100 to provide higher power of the bioenergy for recovery, as experienced by several patients. The bioenergy level of the organs of the user was measured by using a Bioplasm 9D-NLS Bioresonance Machine (9D-NLSBM) (Bioplasm 9D-NLS Bioresonance Machine—Aura Chakra Healing, available at https://www.biophiliatracker.com/Bioplasm-9D). The 9D-NLSBM is widely used in the international bioresearch community. Basically, 9D-NLSBM was designed to provide the most accurate diagnostic results because it focuses on the source, the brain. Therefore, the brain is where anyone begins accumulating biophoton on the changes that occur in the body. The sensors in the headphones placed on the ears of the subject will trigger the brain to communicate the biophoton that occur within the body. Each and every change of the biophoton at the cellular level was detected and compared before and after using a bioenergy generator 100. FIG. 18 to FIG. 24 were the outcome of the measurements by using the 9D-NLSBM.

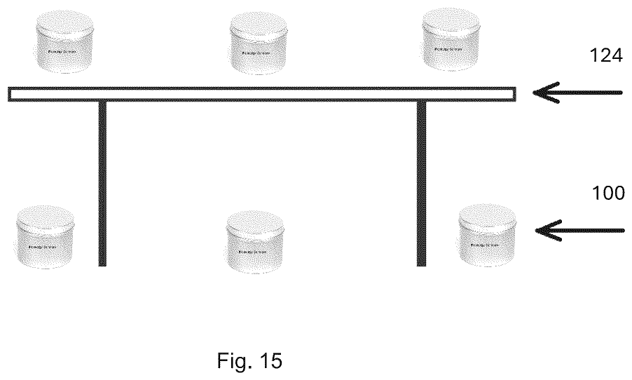

In the embodiment illustrated in FIG. 15, the bioenergy generator 100 can be placed anywhere in a relationship with a table 124. The number and bioenergy strength of the bioenergy generator 100 can be varied depending on the user's need for the bioenergy, such as the different age or health condition. A working person using a bioenergy-empowered table to gain energy would need one or two bioenergy generators for promoting working efficiency. For these who have chronic disease, such as arthritis, would need 2 or more bioenergy generators to provide higher power of the bioenergy for recovery, as experienced by several patients. The bioenergy level of the organs of the user was measured by using a Bioplasm 9D-NLS Bioresonance Machine (9D-NLSBM).

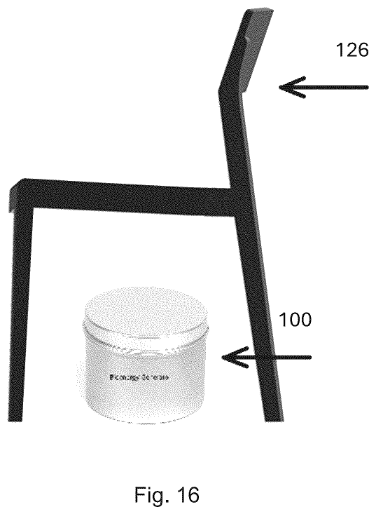

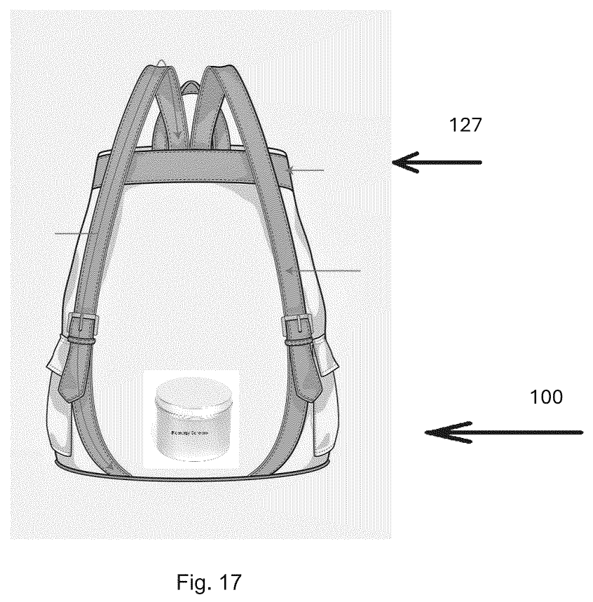

In the embodiment illustrated in FIG. 16, the bioenergy generator 100 can be placed under a chair 126. The number and bioenergy strength of the bioenergy generator 100 can be varied depending on the user's need for the bioenergy, such as a young or an old person. A young adult who uses bioenergy-empowered chair to gain energy would need one bioenergy generator 100 for promoting working efficiency. For these who is over 50 years of age, would need 2 or more bioenergy generators to provide higher power of the bioenergy under a chair or sofa. The bioenergy level of the organs of the user was measured by using a Bioplasm 9D-NLS Bioresonance Machine (9D-NLSBM).

In the embodiment illustrated in FIG. 17, the bioenergy generator 100 can be placed in a backpack 128. The size and number of the bioenergy generator 100 can be varied depending on the user's need for the bioenergy, such as an active adult or a senior person with chronic fatigue. The active adult who uses bioenergy-empowered backpack to gain energy would need one bioenergy generator 100. For these who is over 50 years of age with any chronic health condition, would need 2 or more bioenergy generators. The bioenergy level of the organs of the user was measured by using a Bioplasm 9D-NLS Bioresonance Machine (9D-NLSBM).

In the embodiment illustrated in FIG. 18, is a scan image of the 9D-NLSBM showing the improved cellular activities of the cerebral arteries after an adult who was treated with the bioenergy generator 100 for 4 nights as compared to the baseline condition. The cellular repair rate of cerebral arteries was increased by 39% after using the bed powered with 8 bioenergy generators 100 for the 4 nights. This result indicated that the treatment with a bioenergy generator 100 for four nights significantly improved the cellular activity of the brain arteries. The improved brain arteries can supply the brain nerve tissue with constant blood, so the brain cells can be surely nourished with nutrients and oxygen. Many sub-organs inside of the brain, such as the visual cortex of the brain, could have a better function, or improved vision

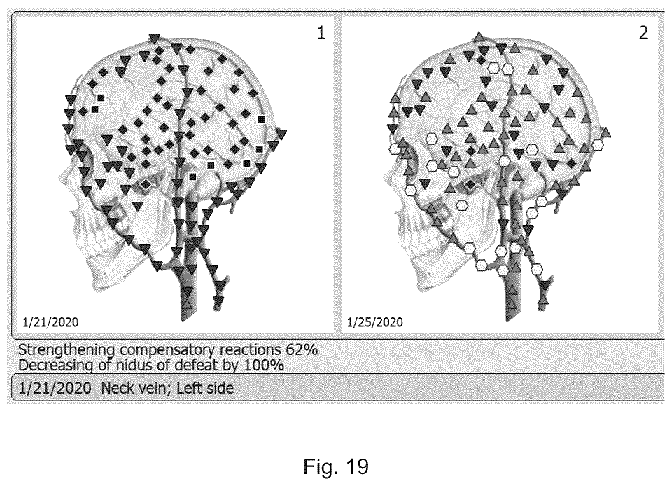

In the embodiment illustrated in FIG. 19, a scan image using the 9D-NLSBM shows the improved cellular activities of the neck vein after an adult who was treated with the bioenergy generator 100 for 4 nights as compared to the baseline condition. The cellular repair rate of the neck vein was increased by 62%. The cellular destruction rate was decreased by 100%. This result indicated that the treatment with a bioenergy generator 100 for four nights significantly improved the cellular activity of the neck vein. The improved neck vein can increase the blood circulation of the brain.

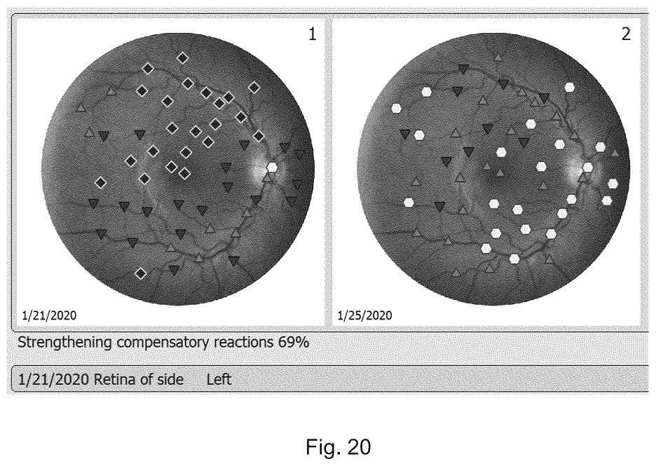

In the embodiment illustrated in FIG. 20, a scan image using the 9D-NLSBM shows the improved cellular activities of the retina after an adult who was treated with the bioenergy generator 100 for 4 nights as compared to the baseline condition. The cellular repair rate of the retina was increased by 69%. This result indicated that the treatment with a bioenergy generator 100 for four nights significantly improved the cellular activity of the retina. The improved retina can increase visual function.

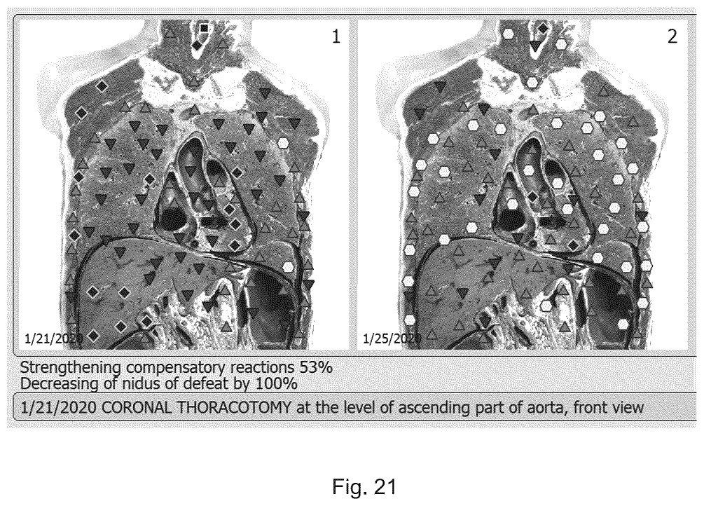

In the embodiment illustrated in FIG. 21, a scan image using the 9D-NLSBM shows the improved cellular activities of the major organs of an adult who was treated with the bioenergy generator 100 for 4 nights as compared to the baseline condition. The cellular repair rate of those organs was increased by 69%. This result indicated that the treatment with a bioenergy generator 100 for four nights significantly improved the cellular activity of the major organs.

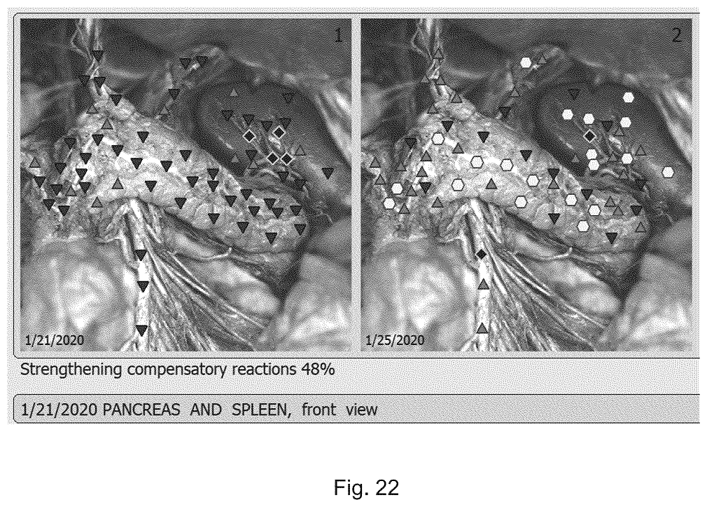

In the embodiment illustrated in FIG. 22, is a scan image using the 9D-NLSBM shows the improved cellular activities of the pancreas and spleen of an adult who was treated with the bioenergy generator 100 for 4 nights as compared to the baseline condition. The cellular repair rate of the pancreas and spleen was increased by 48%. This result indicated that the treatment with a bioenergy generator 100 for four nights significantly improved the cellular activity of the pancreas and spleen. The improved cellular activities of the pancreas and spleen can increase digestion efficiency.

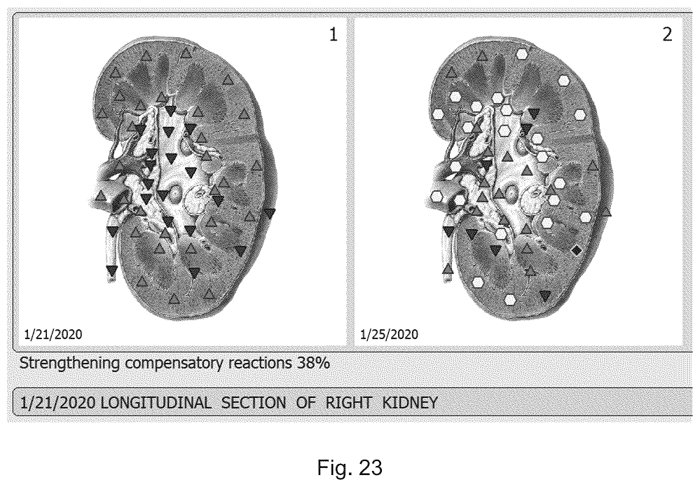

In the embodiment illustrated in FIG. 23, a scan image using the 9D-NLSBM shows the improved cellular activities of the kidney of an adult who was treated with the bioenergy generator 100 for 4 nights as compared to the baseline condition. The cellular repair rate of the kidney was increased by 38%. This result indicated that the treatment with a bioenergy generator 100 for four nights significantly improved the cellular activity of the kidney. The improved cellular activities of the kidney can increase the detoxification capacity of the kidney.

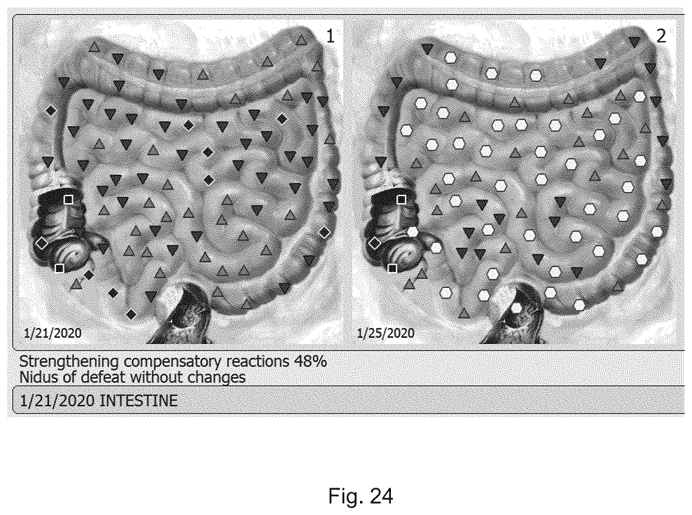

In the embodiment illustrated in FIG. 24, is a scan image using the 9D-NLSBM showing the improved cellular activities of the intestine of an adult who was treated with the bioenergy generator 100 for 4 nights as compared to the baseline condition. The cellular repair rate of the intestine was increased by 48%. This result indicated that the treatment with a bioenergy generator 100 for four nights significantly improved the cellular activity of the intestine. The improved cellular activities of the intestine can increase the absorption rate of the foods.

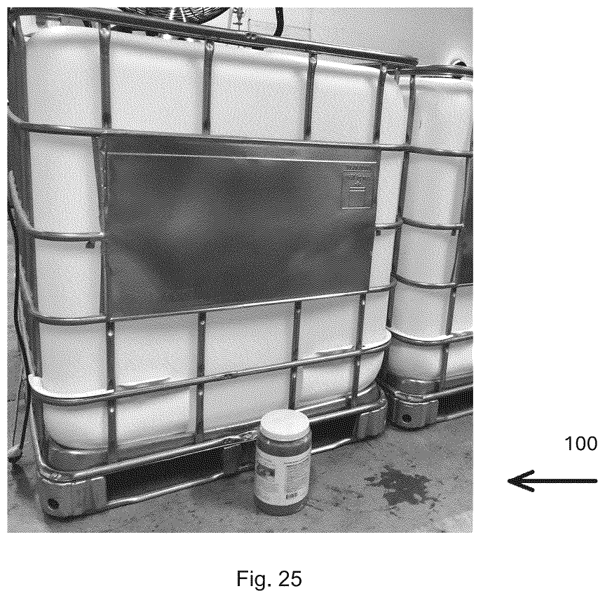

In the embodiment illustrated in FIG. 25, is a photo showing that the bioenergy generator 100 was placed next to a fermentation tank 128 to speed up the fermentation process in making wines using strawberry. The 240-gallon fermentation tank was fully loaded with freshly harvested strawberry. A 12 Kg bioenergy generator 100 was used to re-start the fermentation process. After 8 hours, the air-bubbles were continually flowing out, which indicated that the fermentation inside of 240-gallon tank reactivated.

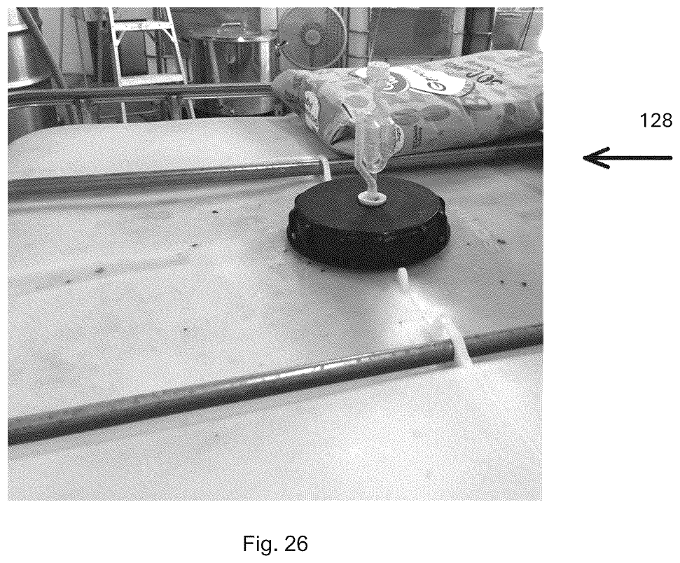

In the embodiment illustrated in FIG. 26 is a photo showing the 240-gallon fermentation tank 128 was bubbling after the bioenergy generator 100 was placed next to the fermentation tank for 8 hours or more. The fermentation process inside of 240-gallon tank was continually active for 2 more days as compared to the same size-tank as the control. The newly added bioenergy generator 100 increased the overall yield of the ethanol production by 10% as measured by the operator.

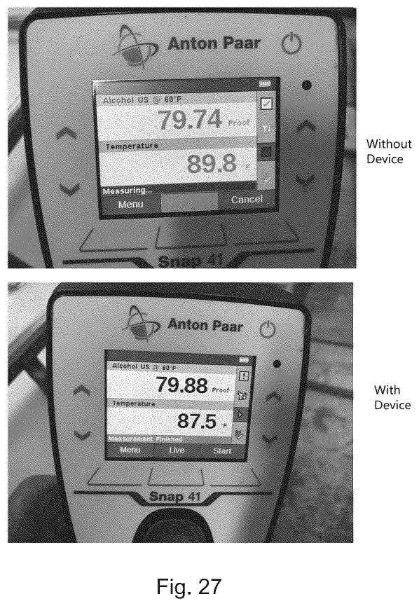

In the embodiment illustrated in FIG. 27, two photos show the alcohol proof number. After the finished beverage was placed next to the bioenergy generator 100, the alcohol proof number was significantly higher than that without the device. A 0.14 proof difference was significant.

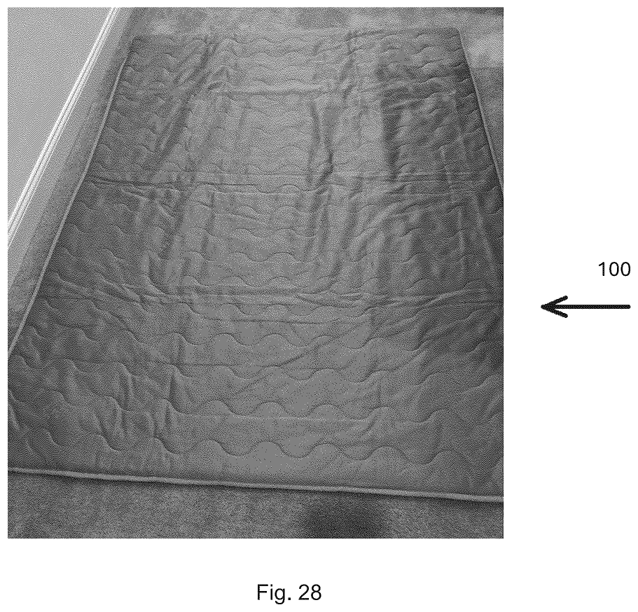

In the embodiment illustrated in FIG. 28 is a pad manufactured with the composition and materials to produce both life force (scalar wave) and infrared wave. The size of the pad can be varied to cover the smaller mattress, like a full bed, or a large mattress, like a king bed.

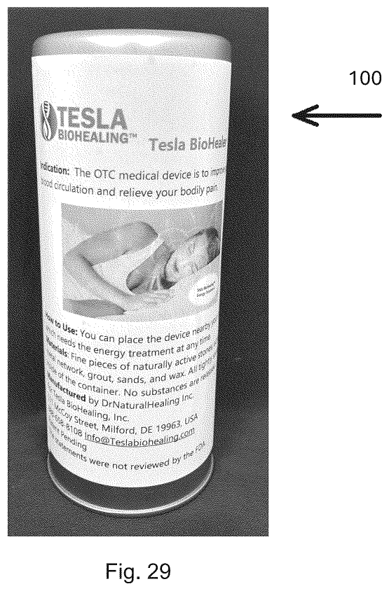

In the embodiment illustrated in FIG. 29, a photo of the bioenergy generator 100 with all materials to produce life force. It shows a supporting structure to hold the fine pieces of natural minerals and a network of a fine metal wire together to consistently generate a life force stronger than the natural minerals and network of a fine metal wire that exists alone.

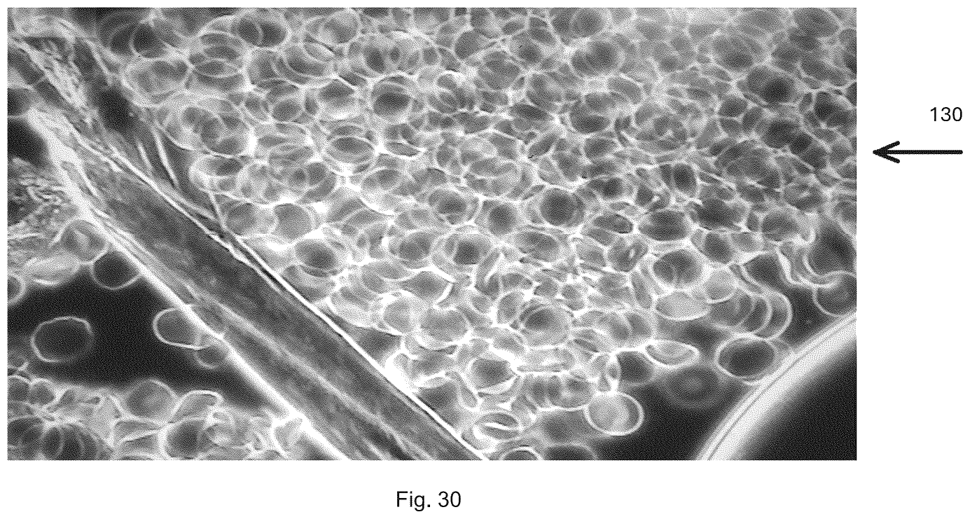

FIG. 30, is a photo of a blood specimen 130 is examined under a microscope. The sample was taken from the Inventor number one who used the bioenergy generator 100s for 4 nights. The fluidity of the blood was significantly improved.

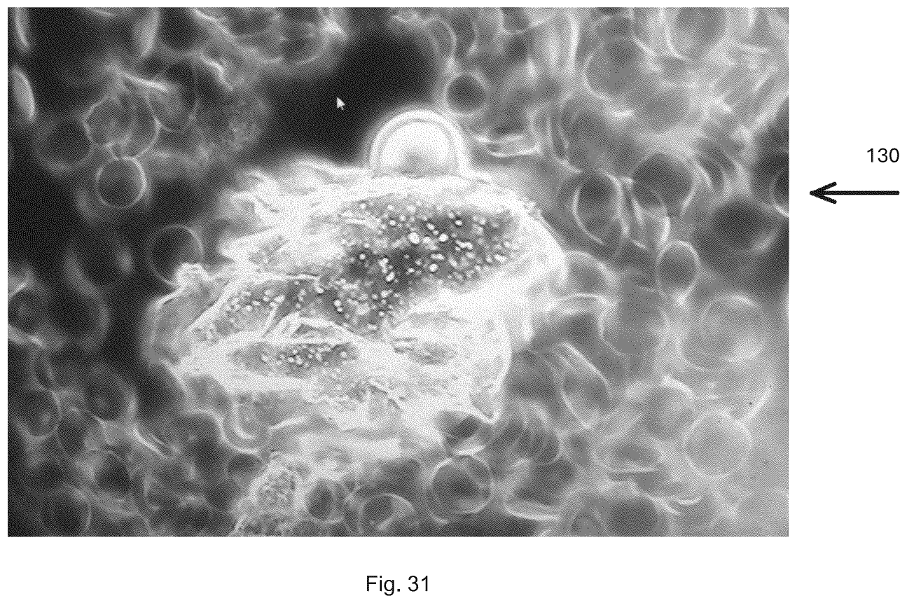

FIG. 31 is a photo of the blood specimen 130 is examined under a microscope. The sample was taken from the first named inventor who used the bioenergy generators 100 for 4 nights. The waste material was dropping off from the blood vessel and floating in the blood, then to be removed from the blood. Removing waste pieces from the blood vessel will make the blood vessel have large internal diameter so it is easier for the blood flow.

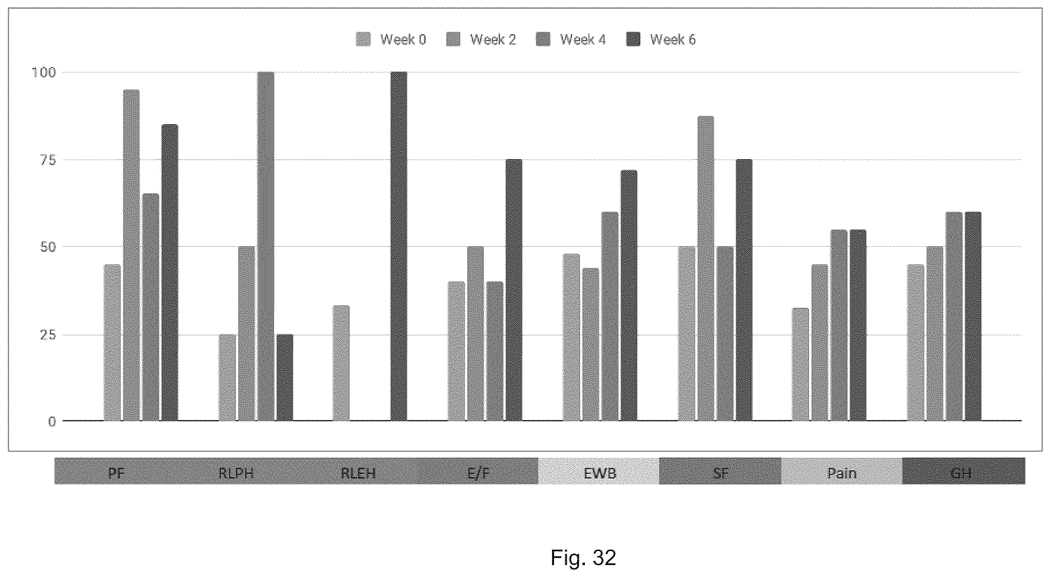

FIG. 32 is an SF-36 questionnaire comparison bar graph for a cancer patient (K. M.) at baseline (0 week) and 4 weeks after used bioenergy generators 100. Seven out of the 8 group SF-36 scores were significantly increased. The full name of the brief group score of the SF-36 respectively are: Physical functioning (PF); Role limitations due to physical health (RLPH); Role limitations due to emotional health (RLEH); Energy/fatigue (E/F); Emotional well-being (EWB); Social functioning (SF); Pain (Pain); General health (GH).

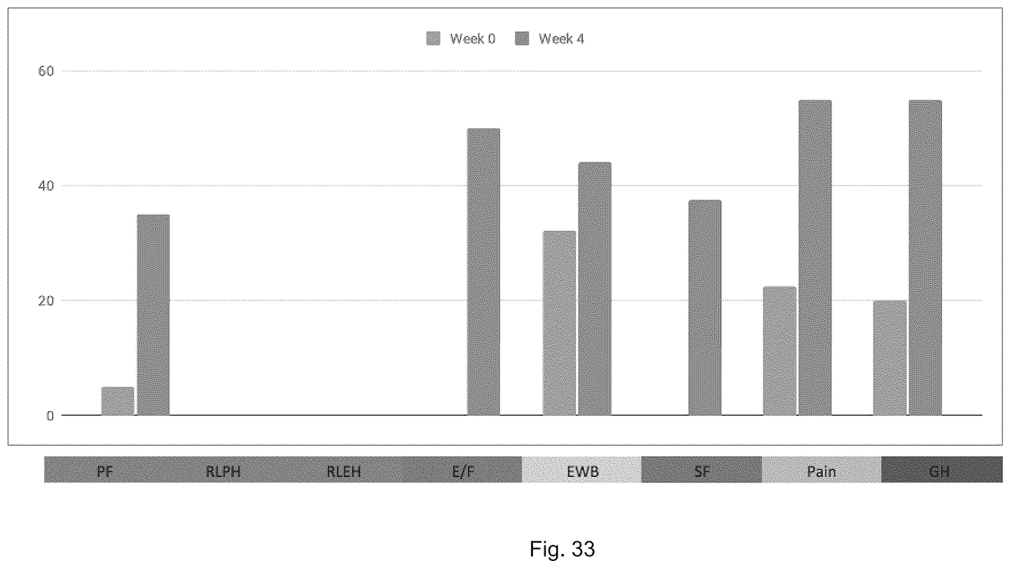

FIG. 33 is an SF-36 questionnaire comparison bar graph for a dementia patient (E. B.) at baseline (0 week) and 4 weeks after used bioenergy generators 100. Five out of the 6 group SF-36 scores were significantly increased. This patient could live independently again.

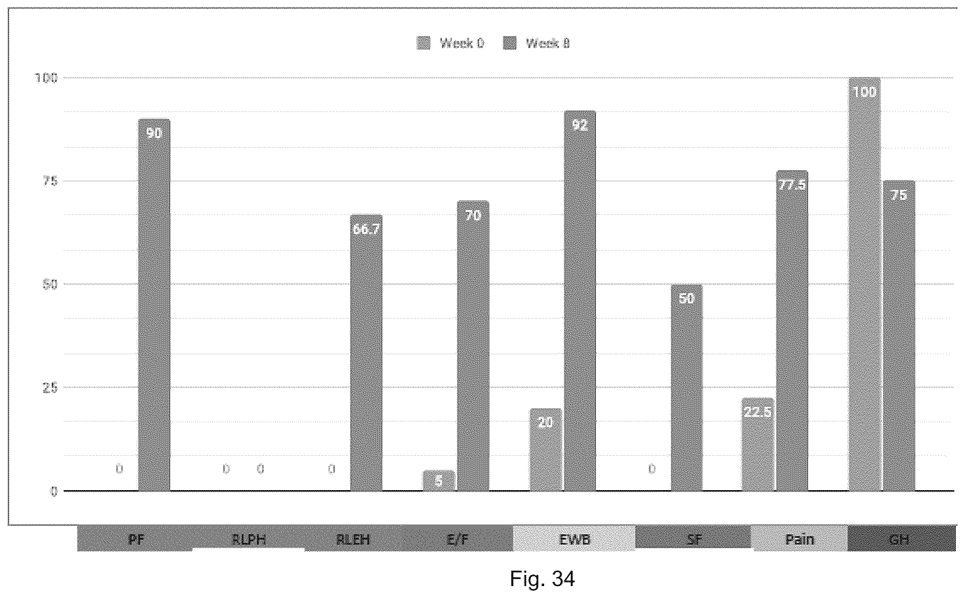

FIG. 34 is an SF-36 questionnaire comparison bar graph for a patient with seizure (C. S.) at baseline (0 week) and 8 weeks after used bioenergy generators 100. Six out of the 7 group SF-36 scores were significantly increased. This patient virtually eliminated the seizure event 8 weeks after using bioenergy generators 100.

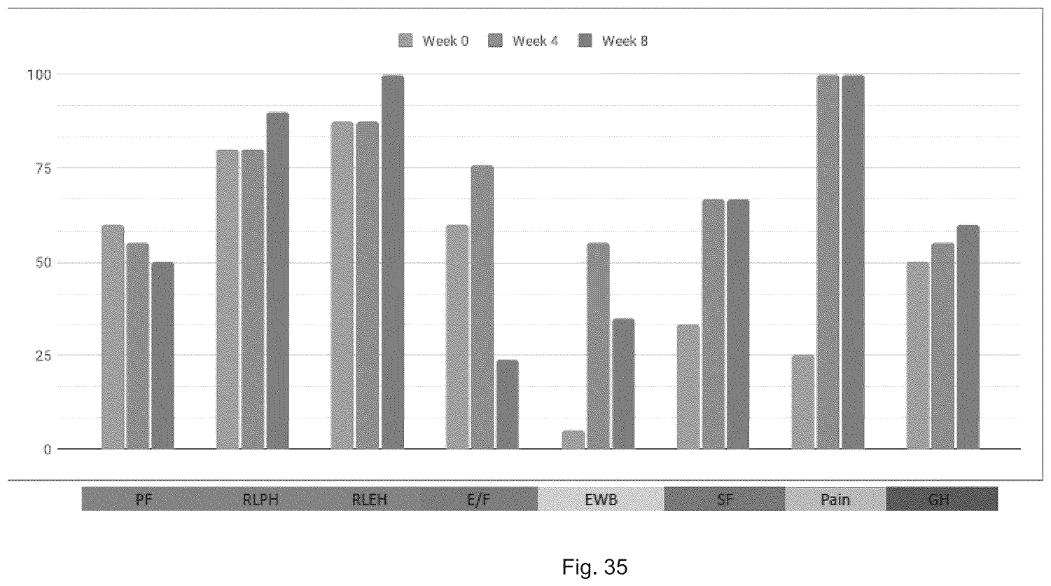

FIG. 35 is an SF-36 questionnaire comparison bar graph for a patient with stroke (C. N.) at baseline (0 week) and 4 weeks after using bioenergy generators 100. Seven out of the 8 group SF-36 scores were significantly increased. This patient regained the mobility from total paralysis for 2 years.

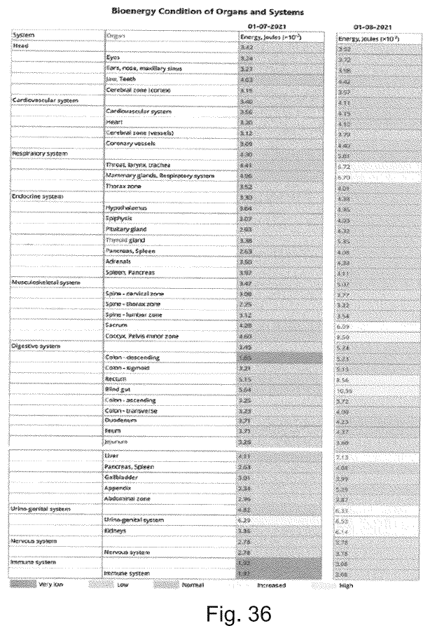

FIG. 36 is a bioenergy comparison for a volunteer with severe back pain after one night use of bioenergy generators 100. For all of the 52 body parts or systems, the bioenergy level was significantly increased. This is the mechanism of action why the bioenergy generators 100 can increase the body energy for empowering the self-healing. The organ bioenergy was measured by using a gas discharge visualization (GDV) device.

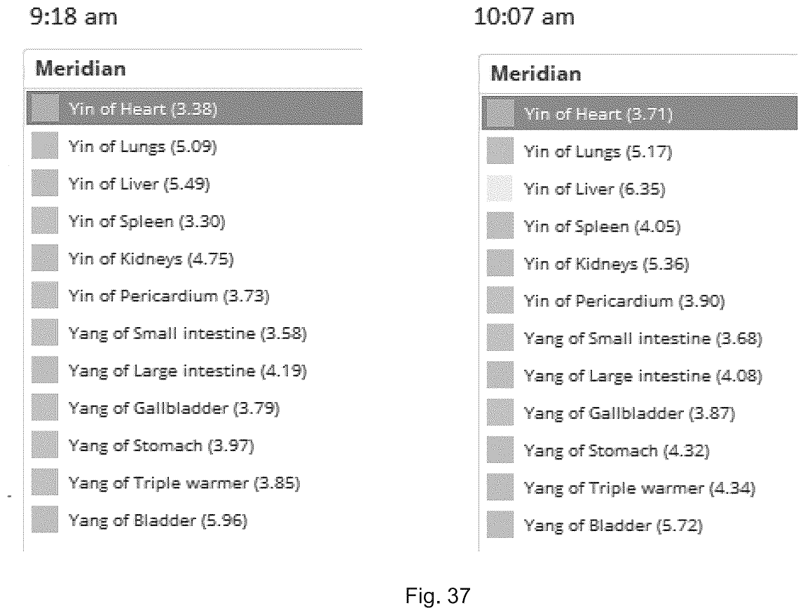

FIG. 37 is a meridian bioenergy comparison for a volunteer (the first named inventor) after using the bioenergy generators 100 for 49 minutes, all of the 12 meridians increased the bioenergy level. This is another mechanism of action why the bioenergy generators 100 can have the broad therapeutic effects. The bio-well is used globally. The increased meridian bioenergy can have a better health, which was the same as acupuncture therapy. The meridian bioenergy was measured by using a gas discharge visualization (GDV) device.

An object of the current invention is to provide a series of medical devices to generate bioenergy to empower a biological system, which is a mammal, a plant, a fermentation process, or a biological body, such as an egg or a tomato, for example. The newly generated bioenergy is not linear radiation, rather, it is a 3-D sphere, as illustrated in FIG. 2. Depending on the source of the bioenergy generator 100, the radius of the bioenergy sphere can be varied in a large range, which is to meet the different needs of any biological system. Bioenergy generator 100 is the core of energy medicine.

The nature of the bioenergy sphere produced by the current invention was a scalar wave, or it can be called Tesla wave, or biofield, or Chi (Qi), or aura. The mechanism of actions of the scalar wave to benefit a biological system are very different from the drugs. Although the effective drug can accurately target the therapeutic area, however, the principle of the drug treatment, on most occasions, was to kill a pathogen or to kill the cancer cells, or suppress the inflammation process. Suppressing cancer cells or inflammatory reactions carries a certain degree of side effects for the normal cells. It is hard for a drug to be a pure benefit to a person. Therefore, an object of the current invention is to provide the life force to meet many people's desire of maintaining or increasing natural health by using the internal stem cell growth method, one of the ideal natural healing approaches. Certain drugs, like growth hormones for promoting stem cell production, need an injection and can have many other risks. Hormone replacement therapy also carries the risk of causing cancers. Energy therapy, or energy medicine, such as acupuncture to adjust the level of Qi on each of the 12 meridians, Qi Gong practice, Tai Qi, are a few examples of the energy therapy used in Traditional Chinese Medicine (TCM) without noticeable long-term side effects. Using these forms of energies required a person with special training to do the treatment. Far-infrared therapy, one of the modern energy medicines, has many beneficial effects when used appropriately. However, infrared is a one-directional ray, not a 3D sphere energy. Nonetheless, it is beneficial to combine scalar wave energy and infrared energy in this invention. The current invention provides the device to generate both infrared energy and scalar wave energy, so they can benefit a biological system synergistically.

An object of the newly invented energy therapy is that the applied energy was to increase the body's innate ability in self-protection and self-defense against pathogens and reduce inflammation. This is particularly important when the body was to win the battle against chronic disorders, such as stroke-related paralysis, dementia, terminal cancer, or post-treatment Lyme disease syndromes. Overall, an object of the current invention is to provide new bioenergy and a new method in the field of energy medicine to enhance self-healing.

The newly invented energy therapy is a continuation of medicines with a long history. Back in 400 B.C., the great Greek physician Hippocrates of Kos told people that everyone had an innate healing system. It is still very important that the innate healing system should be enhanced and utilized for maintaining normal health. Optimum health heavily relies on the innate healing capacity of the body. An object of the current invention is to enhance the body's innate healing ability.

The newly invented energy therapy is also a continuation of Traditional Chinese Medicine (TCM) with more than 4000 years of successful history. Li Shizhen, a well-known physician and pharmacist in the history of the TCM told people that “for body healing, food was better than a drug; water was better than food, sleep was better than water.” Therefore, the importance of a good night's sleep for maintaining health and normal body function was recognized a very long time ago. An object of the current invention is to improve people's sleeping efficiency and to increase the body's energy level.

The operability of the current invention of promoting sleep was supported by modern scientific research. On Nov. 1, 2019, a group of scientists at Boston University and Harvard University reported that only during deep sleep time, the brain can effectively remove the metabolic wastes-such as amyloid-beta. If the metabolic wastes were accumulated, the cognition capacity and brain tissue health would be in question, which could be a risk factor for developing Alzheimer's disease (AD). Based on the current medical science, AD is an irreversible, progressive brain disorder that slowly destroys memory and thinking skills and, eventually, the ability to carry out the simplest tasks. So far, there is no effective therapy for AD. The current invention is operable to reverse the course of AD. The preliminary use-test revealed that the current invention helped the dementia patient to recover and reverse the cognitive capacity.

Several materials can be used to make the scalar wave generators without electricity according to the current disclosure. Steel wool, also known as wire sponge, is a bundle of very fine and flexible sharp-edged steel filaments. It is used safely as an abrasive in finishing and repair work for polishing wood or metal objects, cleaning household cookware, cleaning windows, and sanding surfaces. Steel wool is also a cheap, efficient tool for keeping out those nasty mice and rats from the house, tent, or any other living space. Simply stuff the steel wool into small cracks and small open spaces (or use them to plug a hole, perhaps in the baseboards, pipes, or small entryways) and cover the steel wool with duct tape. The inventors disclosed that steel wool is one of the suitable materials which can be used as one of the components from 1% to 50% of the overall weight to form a bioenergy generator 100 to produce life force. Particularly, the usefulness of the metal wool for generating life force was increased dramatically when it was pre-activated by place the package next to a strong scalar wave generator.

It is disclosed that the fineness of the metal wool impacts the capacity of the new device to generate life force very significantly. On the same weight basis, the finer the metal wool is, the stronger the life force can be generated. Below was the grade of steel wool and the ability to generate life force after mixed with the other component.

| Grade | 0000 | 000 | 00 | 0 | 1 | 2 | 3 | 4 |
|---|---|---|---|---|---|---|---|---|
| Name | Finest | Extra | Fine | Medium | Medium | Medium | Coarse | Extra |
| | | Fine | | Fine | | Coarse | | Coarse |
| Inches | 0.001 | 0.0015 | 0.0018 | 0.002 | 0.0025 | 0.003 | 0.0035 | 0.0047 |
| MM | 0.03 | 0.04 | 0.04 | 0.05 | 0.06 | 0.08 | 0.09 | 0.1 |
| Microns | 25 | 35 | 40 | 50 | 60 | 75 | 90 | 100 |
| Life | Highest | Very | High | Low | Lower | Weak | Very | Weakest |
| Force* | | High | | | | | Weak | |
| Generated | | | | | | | | |

The levels of life force generated are as follows: Highest: If the net increase in life force by at least 100K units; Very High: If the net increase in life force by no less than 50K units; High: If the net increase in life force by no less than 30K units; Low: If the net increase in life force by no less than 20K units; Lower: If the net increase in life force by 10K units; Weak: If the net increase in life force by 5K units; Very Weak If the net increase in life force by only 2K units; and Weakest: If the net increase in life force by only 1K units.

The stainless steel wool has a similar capacity to steel wool for being used as a component to generate life force after being mixed with the other essential component. The finer the stainless steel wool is, the stronger the life force is generated.

The copper wool has a similar capacity to steel wool for being used as a component to generate life force after being mixed with the other essential component. The finer the copper wool is, the stronger the life force is generated.

The other metal wools can be used, and if the life force is high enough (more than 12K units) and the right fineness is acceptable, the material can be used to make the device generate life force. Metal wires, including copper wire, or copper coils, can be used for the life force units. After each component was tested and accepted, it can be used to make the device generate life force.

Several bioactive solids varied in size, or fineness, such as colorful sands, or small stones, which can be used to make the life force generators 100. Minerals are solid chemical compounds that occur naturally and were tested as having the bioenergy not less than 10K units. Minerals are most commonly associated with sands and rocks due to the presence of minerals within sands and rocks. These sands and rocks may consist of one type of mineral or may be an aggregate of two or more different types of minerals, spatially segregated into distinct phases. Compounds that occur only in living beings are usually excluded from the sands or stones after deposition and evaporation for thousands of years, but some minerals are often biogenic or are organic compounds in the sense of chemistry. Moreover, living beings often synthesize inorganic minerals that also occur in rocks. The inventors discovered and disclosed that certain minerals are suitable materials for use to make the device generate life force in combination with the other components. They can be used to make a serial of devices to provide certain levels of life force. Those devices can be used for a variety of purposes, such as to promote sleep or treat a disorder. Particularly, the usefulness of the bioactive solids for generating life force was increased dramatically when it was pre-charged with a strong scalar wave source.

The grading and maximum size of colorful sands are important parameters in making the devices for generating life force. They affect relative proportions in mix, workability, economy, porosity, and shrinkage of the content of the solid to generate life force. Our experience has shown that very fine sands produced the highest life force when the other essential components were fixed. The very coarse colorful sands produced less life force. The fineness modulus of colorful sands and their ability to produce life force was listed below.

| Sieve Size | | Life Force Production |
|---|---|---|
| (Fineness) | | Capacity |
| 4.75 | mm | Not Strong, Less than 5K Units |
| 2.36 | mm | Less Strong, ≥5K Units |
| 1.18 | mm | Strong, >10K Units |
| 0.6 | mm | Stronger, >25K Units |
| 0.3 | mm | Very Strong, >50K Units |
| 0.15 | mm | Strongest, >100K Units |

Bioactive sands have many colors, such as white, black, pink, red, orange, green. After testing for the life force units, these with at least 10K units measured with a Bovis Life Force Scaler. The color of sand provides clues as to its makeup. Sand's color is derived from its mineralogy or the physical structure of the crystals that populate the sand. These minerals can come from erosion of nearby landscape, volcanic eruptions, and even the grounding up of seashells over decades, so the color and content of sand reflect the makeup of the surrounding landscape. The natural color sands, after being screened to have a good level of life force units, were used in the current invention.

Amethyst can be used as a material to generate a strong life force. The original ingredient of amethyst was tested to have a low level of life force. It can be activated by using a strong lifeforce generator. It is a good material for being used to make the bioenergy sphere generator with the other components. Amethyst has been used as a healing stone for thousands of years and is a great all-purpose healing stone. This is largely due to its ability to generate far-infrared rays and negative ions as reported in the literature.

Amethyst is a superconductive gemstone and generates a small magnetic field. Amethyst can also generate infrared rays that penetrate the body 6-8 inches to stimulate cellar metabolism and detoxification. Amethyst can induce Delta/Theta brain waves to promote deep relations, meditation, and restorative sleep. Metaphysically amethyst has been known to stimulate and soothe the mind and emotions. It activates the “crown chakra” and “third eye chakra” which are the gateways to expand oneself spiritually and to the expanded universe beyond our bodies. These chakras are largely connected to the dream state which may be why amethyst is considered an excellent stone to assist in sleeping. However, Amethyst itself only has a very low level of life force. When it was used with the other components, the new mixture generated 10 or even 100 times more life force units as disclosed herein.

Tourmaline can be used as a material to generate a strong life force. Tourmaline was tested to be a good material for being used to make the bioenergy sphere generator 100 with the other components. Tourmaline itself can produce negative ions when it was heated up or at room temperature. For example, tourmaline produces 1500+ negative ions per cubic centimeter when heated. Tourmaline is known as the electric stone as it produces natural piezoelectric currents which are partly the source of the stones naturally generated negative ions. Tourmaline is superconductive and generates a natural Infrared wave. Tourmaline radiates energy at 8-13 Hz per second which is the identical frequency as the brain's alpha waves. This can bring a person's mind into an alpha state much quicker than normal. The alpha state of the brain can reduce anxiety, bring calmness, increase energy while reducing fatigue, improve mood, and is ideal for mediation. Tourmaline counteracts the effects of unnatural/high-frequency electromagnetic fields through its release of negative ions. Metaphysically tourmaline is known as a stone good for grounding and healing oneself. It helps create a connection between one's base chakra and the earth. When the base chakra and earth connection are out of balance individuals frequently will find themselves lethargic, unmotivated, unenthusiastic, and in need of constant stimulation. By strengthening the base chakra and ground connection the physical body can regain its strength & stamina. However, Tourmaline itself only provides a very low level of life force. When it was used with the other components, the new mixture generated 10 or even 100 times more life force units than the raw material used alone in the current invention.

Jade stone can be used as a material to generate a strong life force. Jade stone was tested as a good material for being used to make the bioenergy sphere generator with the other components. It is known as one of the best natural heat absorbers. Adding jade stone to amethyst and tourmaline enhances their natural attributes such as increasing negative ion production and producing Far Infrared Rays. When left unheated jade stone can also be used to provide a relaxing coolness. When heated jade stone typically produces around 700 negative ions per cubic centimeter. Metaphysically Jade stone opens the heart chakra which promotes love, compassion, forgiveness, and trust. When the heart chakra is blocked it can lead to grief, depression, loneliness, and fear. Different jade stones have special usefulness. Black Jade can be effectively used to help protect against viral and bacterial infection, parasitic infestation, and associated illnesses. It is excellent to use when traveling. Blue Jade assists in calming inflammation, swelling, arthritic conditions, asthma, and bronchial conditions. Green Jade is generally strengthening the energy systems, but there are distinct differences in the physical effects of Nephrite and Jadeite. Green Nephrite Jade is a powerful physical heart healer. It also helps strengthen and soothe the nervous system. Green Jadeite offers support in detoxification and the regeneration of the tissues after trauma or surgery. Orange Jade stone benefits the skin encouraging the healing of skin diseases such as eczema, dermatitis, and other allergy-related skin conditions. This stone will boost and cleanse the circulatory system. Purple Jade helps soothe the nervous system. It can assist those who suffer from hives, rashes, or outbreaks that are exacerbated by stress. Red Jade is a tonic stone—an excellent invigorator of all the systems of the body. All of these jade stones can be used as one of the components to make the current invention generate a huge amount of life force. The newly produced life force with jade stone is at least 2 times stronger than the jade stone alone, or even 80 times higher.

Obsidian was tested to be a good material for being used to make the bioenergy sphere generator with the other components. The Obsidian crystal stone can be traced back to Paleolithic times where it was used to make arrowheads and other tools. Cherished for its deep, glossy sheen, Obsidian is often called volcanic glass because it is formed from molten lava that has cooled very quickly. Obsidian has the effects of amplifying the properties amethyst, tourmaline, and jade, it is a natural pain reliever, it blocks geopathic stress and environmental pollution, it is an emotional healing stone. Metaphysically Obsidian is a very protective stone and is excellent for removing negative energy on the physical level (pain) and the emotional level (depression). It clears the psychic smog from the aura while protecting one from psychic attacks. It grounds the patient to the earth through the base chakra and repels unloving thoughts. The newly produced life force with Obsidian is at least 2 times stronger than Obsidian alone, or even 80 times higher.

The other sands and stones can be tested, and if the life force is high enough, and the right particle size can be obtained after a special process.

The manufacturing process of the current invention was disclosed hereunder. Step 1 is to test the life force of raw materials. Step 2 is to weigh each of the raw materials. Step 3 is to mix the weighed raw materials in a container. Step 4 is to put the mix into a container. Step 5 is to seal the container tightly. Step 6 is to measure the life force for each device. Step 7 is to add a label to the container. Step 8 is to place each finished device in the shipping case.

It is disclosed that the fineness of the above specified bioactive stones impacts the capacity of the bioenergy sphere 100 to generate life force significantly. On the same weight basis, the smaller the bioactive stone is, the stronger the life force can be generated.

In accordance with embodiments of the invention, a bioenergy generator 100 generates and releases the life force to empower the body's cells for promoting the health and function of the body. This bioenergy generator 100 has been test-used and remarkable results were observed.

The new therapeutic effects of bioenergy generators 100 were observed after it was used to treat dementia and other health conditions. All those who used the new device improved the body function meaningfully. The newly invented biofield is verified to be beneficial to a man, a woman, a young, an old, and anyone in between. These therapeutic effects are important parts of the claims of the current invention. This invention has a very broad therapeutic effect and a high level of safety. No side effects were observed from any users.

The bio-recharge system can be called a quantum cell activator. After the use-tested of the current invention, the inventors observed numerous excellent effects from many in the past. The quantum-cell activator provided by the bioenergy generator 100 did not only restore the users' energy level but also improved the sub-health constitution. More impressionably, the patients with high blood pressure, diabetes, terminal cancer, insomnia, depression, heart failure, and osteoporosis, recovered to a very satisfactory level. Many users' physical and mental exhaustion was significantly improved, which were also parts of the therapeutic effects. Those elderly who were physically weak and unable to take care of themselves were significantly improved and could self-support for daily life. The below examples are to summarize the therapeutic effects experienced by many patients and users.

The life force generated by using the current invention has numerous applications, such as:
Treat or cure human diseases, such as Alzheimer's Disease.
Correct or cure a human disorder, such as hypertension.
Produce stem cells naturally to the human body to promote health.
Beautify human skin, including facial skin.
Increase the cognitive capability of a human, particularly for those who have a low intelligence level, such as a low IQ.
Reverse the lost human cognitive capability, particularly for those with dementia.
Increase body growth for a growing human body, particularly for the child who has a short stature.
Increase the metabolism of a human, particularly for the one with a poor digestion capacity.
Improve blood circulation, particularly for those who have diabetes.
Increase immunity for a human, particularly for those who are easy to get an infection.
Increase wound healing speed, particularly for those who have surgery.
Increase energy level, particularly for those who need physical strength.
Increase sleeping efficiency, particularly for those who have poor sleep.
Reduce inflammation, particularly for those who have arthritis.
Improve hormone balance, particularly for those who have a hypothyroid function.
Improve Chi (Qi, or aura), particularly for those who are lack Chi based on traditional Chinese medicine.
Improve mood status, particularly for those who have a mental disorder.
Improve bone health, particularly for those who have osteoporosis.
Use bioenergy to charge these regular hospitals or clinic beds to promote a patient's recovery.
Use bioenergy to charge these regular beds or sofa to promote people's health during the night.
Use bioenergy to charge these regular chairs and tables to promote people's health during working time and to increase productivity.
Use bioenergy to charge this regular backpack or any carrying tool to promote the user's health at any time.
Use bioenergy to charge these regular rooms or spaces to promote people's health at any time.
Use bioenergy to charge these conference rooms to promote people's health during the meeting and so increase the meeting's efficiency.
Use bioenergy to charge these transportation tools to be the bioenergy charged tools to promote people's health during traveling.
Use bioenergy to upgrade any facility to promote people's health and happiness.
Use bioenergy to upgrade the training facility for these sportsmen or sportswomen to improve their energy and health for better competition.
Use bioenergy to upgrade the training facility for these artists to improve their energy and performance.
Use bioenergy to upgrade the classrooms for these students to have energy and to focus on what they are taught.
Use bioenergy to upgrade the training facility for these soldiers to have more energy and to be healthier.
Use bioenergy to upgrade the living center for these seniors to have more energy and to improve health.
Use bioenergy to upgrade the daycare center for these children to improve immunity and to have less infection.
Treat or cure an animal disease, such as Lyme disease.
Correct or cure an animal disorder, such as arthritis.
Produce stem cells naturally by an animal body to promote the animal's health.
Increase the bioenergy of animals for competition and work productivity.
Increase the growth of farmer animals so more meat can be produced.
Increase the life force of meat so the freshness can become longer.
Increase the life force of hens so they can lay more eggs.
Increase the life force of eggs so that the eggs can be fresh longer.
Facilitate the growth of fruits and vegetables.
Increase the life force of plants so the yield of plants can be increased.
Keep and increase the life force of fruits and vegetables to be fresh for a longer time.
Maintain and increase the life force of flowers for a longer life span.
Increase the life force of vitamin and mineral products so their health benefit is increased.
Increase the life force of processed food so their health benefit is increased.
Increase life force for soft drinks, such as orange juice.
Increase fermentation speed so more alcohol can be produced.
Speed up the aging process so the wine yield can be increased significantly.
Increase biological process so the yield of the big molecule can be increased for the pharmaceutical industry.
Increase fermentation speed so more large molecules can be produced.

Lifeforce generators can have many other applications. Where there is life, there is a need to use a life force generator. Where there is a biological process, there is a need to use a life force generator.

One general aspect of the present disclosure relates to a method of generating biophotons to enhance natural healing ability of a biological body, wherein the method comprises:
a) selecting and using a mixture of active stones comprising at least two types of stones with distinct electromagnetic properties, such as amethyst, tourmaline, jadestone, obsidian, and diamond;
b) selecting and using fine pieces of natural sand selected from the group consisting of white sand, black sand, pink sand, red sand, yellow sand, orange sand, and green sand;
c) selecting and using fine pieces of a metal, such as iron, copper, zinc, aluminum, calcium, magnesium, silver, and gold;
d) selecting and using water, such as natural water or energized water;
e) selecting and using a supporting material, such as mortar, to hold said natural stones, said natural sand, said metal, and said water together to form a stable solid mixture;
f) arranging said solid mixture in proximity to the biological body;
g) letting said biological body capture biophotons released from said solid mixture; and
h) allowing said biological body to utilize said biophotons to get the benefits of said biophotons.

According to embodiments of the present disclosure, such as FIGS. 18-24, the bioenergy generator described herein can improve cellular activities of a variety of tissues and organs, including cerebral arteries, neck vein, retina, aorta, pancreas, spleen, kidney, and intestine. The cellular repair rate is increased, which indicates that the cellular activity is improved. In addition, the cellular destruction rate is decreased. Therefore, the above results describe a method of generating biophotons to enhance natural healing ability of a biological body.

In some embodiments, the weight percentage of said natural stones, said natural sand, said metal, said water, and said supporting material is from 1% to 95%, relative to the total weight of said solid mixture.

In certain embodiments, the weight percentage of said natural stones is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said natural sand is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said metal is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said water is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said supporting material is between 20% and 96%, such as 20%, 30%, 40%, 50%, 60%, 70%, 80%, 90%, or 96%, or any number in between thereof, relative to the total weight of said solid mixture.

In some embodiments, the method further comprises selecting and using a container to hold said stones, said sand, said metal, said water, and said supporting material together.

In certain embodiments, the container is a stainless steel container or sate plastic container.

In certain embodiments, the container holds said stones, said sand, said metal, said water, and said supporting material together for at least 1 day, 2 days, 3 days, 4 days, 5 days, 6 days, or 7 days.

In some embodiments, said biological body is a human, an animal, or a plant.

According to embodiments of the present disclosure, said natural healing ability can increase cellular repair rate, decrease cell destruction rate, promote cell metabolism, detoxify toxins or free radicals, remove waste products from the cells, and bio-recharge cells to the full voltage level for maintaining optimum health of a biological body. All cells and all organs in the whole body become energized.

In some embodiments, said natural healing ability has a strong cellular repair capacity of said biological body.

In some embodiments, said natural healing ability has a strong cellular capacity against a harmful factor to damage cells of said biological body.

In some embodiments, said natural healing ability has a strong cellular capacity against unhealthy cells of said biological body.

In some embodiments, said natural healing ability has more energized cells to replace injured or aged cells of said biological body.

Another general aspect of the present disclosure relates to a method of generating biophotons to treat a disease in a mammal, the method comprises:
a) selecting and using a mixture of active stones comprising at least two types of stones with distinct electromagnetic properties, such as amethyst, tourmaline, jadestone, obsidian, and diamond;
b) selecting and using fine pieces of natural sand selected from the group consisting of white sand, black sand, pink sand, red sand, yellow sand, orange sand, and green sand;
c) selecting and using fine pieces of a metal, such as iron, copper, zinc, aluminum, calcium, magnesium, silver, and gold;
d) selecting and using water, such as natural water or energized water;
e) selecting and using a supporting material, such as mortar, to hold said natural stones, said natural sand, said metal, and said water together to form a stable solid mixture;
f) arranging said solid mixture in proximity to the mammal;
g) letting said mammal capture biophotons released from said solid mixture; and
h) allowing said biophotons treat the disease.

According to embodiments of the present disclosure, the biophotons generated by the bioenergy generator described herein can facilitate energy-recharge of the cells in any part of a biological body. The biophotons improve health conditions of said biological body and treat many diseases, such as chronic obstructive pulmonary disease (COPD), asthma, hypertension, diabetes, post-COVID, lack or decrease of libido, aging, pain, cancer, side effects of cancer chemo or radiotherapy, uncontrolled glaucoma, Alzheimer's disease, dementia, myasthenia gravis, arthritis, and congestive heart disease.

In some embodiments, the weight percentage of said natural stones, said natural sand, said metal, said water, and said supporting material is from 1% to 95%, relative to the total weight of said solid mixture.

In certain embodiments, the weight percentage of said natural stones is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said natural sand is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said metal is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said water is between 1% and 20%, such as 1%, 2%, 3%, 4%, 5%, 6%, 7%, 8%, 9%, 10%, 11%, 12%, 13%, 14%, 15%, 16%, 17%, 18%, 19%, or 20%, or any number in between thereof, relative to the total weight of said solid mixture.

In certain embodiments, the weight percentage of said supporting material is between 20% and 96%, such as 20%, 30%, 40%, 50%, 60%, 70%, 80%, 90%, or 96%, or any number in between thereof, relative to the total weight of said solid mixture.

In some embodiments, said mammal is a human such as a patient, or a domesticated animal.

In some embodiments, said disease is a brain disorder, a respiratory tract disorder, an endocrinologic disorder, a cardiovascular disorder, a malignant disease such as cancer, pain, uncontrolled glaucoma, myasthenia gravis, or arthritis.

In certain embodiments, said disease is a brain disorder, such as Alzheimer's disease, dementia.

In certain embodiments, said disease is a respiratory tract disorder, such as a chronic obstructive pulmonary disease (COPD), asthma, post-COVID.

In certain embodiments, said disease is an endocrinologic disorder, such as diabetes.

In certain embodiments, said disease is a cardiovascular disorder, such as hypertension, congestive heart disease.

In certain embodiments, said disease is a malignant disease, such as prostate cancer.

In certain embodiments, said disease is a malignant disease, such as breast cancer.

In certain embodiments, said disease is a malignant disease, such as lung cancer.

In certain embodiments, said disease is a malignant disease, such as colon cancer.

In certain embodiments, said disease is a malignant disease, such as skin cancer.

In certain embodiments, the disease is uncontrolled glaucoma.

In certain embodiments, the disease is myasthenia gravis.

In certain embodiments, the disease is arthritis.

In certain embodiments, the method reduces cancer size, reduces cancer pain, and/or reduces side effects of cancer chemo or radiotherapy.

In certain embodiments, the method reduces severe pain.

In certain embodiments, the method increases libido.

In certain embodiments, the method promotes younger looking.

In one another general aspect, provided herein is a method of preventing or mitigating a side effect of a cancer treatment in a subject, the method comprising:
(a) placing one or more biophoton generators close to the subject within a distance; and
(b) keeping the one or more biophoton generators close to the subject daily over a period of time;
   wherein the subject is either scheduled to receive or is receiving the cancer treatment.

As used herein, the term “cancer treatment” refers to any cancer treatment known in the field. Examples include, but are not limited to, radiotherapy, chemotherapy, or targeted therapy.

As used herein, the side effect of the cancer treatment can be any side effect known in the field. Examples include, but are not limited to, pancytopenia, fatigue, pain, nausea, vomiting, hair loss, anemia, weight loss, and organ toxicity.

In some embodiments, the method further enhances the adherence of the cancer treatment, improves the efficacy of the cancer treatment, and/or improves the subject's life quality. As to the improvement of life quality, the method can lead to better sleep, reduced pain, reduced stress, improved energy and vitality, and normalization of health parameters (e.g., easy to go to work and able to help the family).

In some embodiments, the subject has a solid tumor, such as breast cancer, kidney cancer, lung cancer, prostate cancer, colon cancer, skin cancer, brain cancer, gastrointestinal stromal tumor, esophageal carcinoma, cervical cancer, endometrial cancer, ovarian cancer, or bone sarcoma, or any solid tumor known in this field.

In some embodiments, the subject has blood cancer, such as leukemia, lymphoma, or multiple myeloma.

According to embodiments of the present disclosure, the use of the biophoton generators does not reduce the efficacy of the cancer treatment, or at least does not significantly reduce the efficacy of the cancer treatment.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the biophoton generator is kept close to the subject for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the biophoton generator is kept close to the subject for at least 8 hours per day.

In some embodiments, the distance in step a) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present disclosure, the biophoton generator can be positioned at any location that is close to the subject at any direction of the 3-dimensional space of the biophoton generator, and can be used any time of the day, such as daytime or night time, whenever the subject is awake or asleep. The biophoton generator can be used continuously or intermittently, and the use can commence at any time of the day.

In some embodiments, the period of time in step b) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

In some embodiments, the period of time in step b) is longer than, shorter than, or equal to, the duration of the cancer treatment.

In some embodiments, the method can be initialed during any stage of the cancer treatment. For example, the method can be initialed before the cancer treatment, early stage of the cancer treatment, or later stage of the cancer treatment.

In one another general aspect, provided herein is a method of treating cancer in a subject in need thereof, the method comprising:
(a) administering a standard cancer treatment to the subject;
(b) placing one or more biophoton generators close to the subject within a distance; and
(c) keeping the one or more biophoton generators close to the subject daily over a period of time.

As used herein, the term “standard cancer treatment” refers to any cancer treatment known in the field. Examples include, but are not limited to, radiotherapy, chemotherapy, or targeted therapy.

According to embodiments of the present disclosure, the method can reduce the side effect of the standard cancer treatment. The side effect of the cancer treatment can be any side effect known in the field. Examples include, but are not limited to, pancytopenia, fatigue, pain, nausea, vomiting, hair loss, anemia, weight loss, and organ toxicity.

In some embodiments, the method further enhances the adherence of the cancer treatment, improves the efficacy of the cancer treatment, and/or improves the subject's life quality.

In some embodiments, the subject has a solid tumor, such as breast cancer, kidney cancer, lung cancer, prostate cancer, colon cancer, skin cancer, brain cancer, gastrointestinal stromal tumor, esophageal carcinoma, cervical cancer, endometrial cancer, ovarian cancer, or bone sarcoma, or any solid tumor known in this field.

In some embodiments, the subject has blood cancer, such as leukemia, lymphoma, or multiple myeloma.

According to embodiments of the present disclosure, the use of the biophoton generators does not reduce the efficacy of the standard cancer treatment, or at least does not significantly reduce the efficacy of the standard cancer treatment.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the biophoton generator is kept close to the subject for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the biophoton generator is kept close to the subject for at least 8 hours per day.

In some embodiments, the distance in step b) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present disclosure, the biophoton generator can be positioned at any location that is close to the subject at any direction of the 3-dimensional space of the biophoton generator, and can be used any time of the day, such as daytime or night time, whenever the subject is awake or asleep. The biophoton generator can be used continuously or intermittently, and the use can commence at any time of the day.

In some embodiments, the period of time in step c) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

In some embodiments, the period of time in step c) is longer than, shorter than, or equal to, the duration of the standard cancer treatment.

In one another general aspect, provided herein is a method of treating cancer in a subject in need thereof, the method comprising:
(a) placing one or more biophoton generators close to the subject within a distance; and
(b) keeping the one or more biophoton generators close to the subject daily over a period of time.

In some embodiments, the method further improves the subject's life quality, such as better sleep, reduced pain, reduced stress, improved energy and vitality, and normalization of health parameters.

In some embodiments, the method further improves the subject's anemia.

In some embodiments, the subject has a solid tumor, such as breast cancer, kidney cancer, lung cancer, prostate cancer, colon cancer, skin cancer, brain cancer, gastrointestinal stromal tumor, esophageal carcinoma, cervical cancer, endometrial cancer, ovarian cancer, or bone sarcoma, or any solid tumor known in this field.

In some embodiments, the subject has blood cancer, such as leukemia, lymphoma, or multiple myeloma.

According to embodiments of the present disclosure, the use of the biophoton generators does not reduce the efficacy of the cancer treatment, or at least does not significantly reduce the efficacy of the cancer treatment.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the biophoton generator is kept close to the subject for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the biophoton generator is kept close to the subject for at least 8 hours per day.

In some embodiments, the distance in step a) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present disclosure, the biophoton generator can be positioned at any location that is close to the subject at any direction of the 3-dimensional space of the biophoton generator, and can be used any time of the day, such as daytime or night time, whenever the subject is awake or asleep. The biophoton generator can be used continuously or intermittently, and the use can commence at any time of the day.

In some embodiments, the period of time in step b) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

### EXAMPLES

The following examples are provided to illustrate certain aspects of bioenergy generator 100 and to aid those of ordinary skill in the art in practicing the invention. These examples are not intended to limit the scope of the invention.
Example 1. Increased bioenergy level. After used the bioenergy generator 100 for 9 days, a 70-year old lady reported to Dr. James Liu that “I ALSO thank you so very much for the Tesla device that you gave to each of us and to let you know what I have already noticed from using it: Increased energy; Improved mood when waking up (hopeful vs. what is sometimes a bit sad); Improved skin on face—tighter and more radiant; Fast healing of a cut that I had on my face; Vivid dreams every night (I have rarely remembered a dream in the past); Improved sleep—sleeping more deeply and waking up feeling much more refreshed.” The patient utilized a 1 kg bioenergy generator 100.

Example 2. Speed up injury recovery. A senior gentleman aged 71 years wrote to Dr. James Liu: I have a story to share with you that may have to do with your Jar of Energy. Saturday morning about 6:45 AM I was on my way to the Milford office. I had been having trouble with the steering on an old delivery pickup I was driving but had it into the garage for servicing and they told me that they could not find anything wrong with it. I was navigating a turn from Marshall Street to McCoy Street over the bridge and the wheel was locked in a left turn position and I could not move the steering wheel. It was locked in a left turn position. Anyway, due to the rains, we had on Friday I skidded off the road and ended up in the creek in 6′ of water. With the front of the truck submerged I climbed out the window and got to the bed of the truck and jumped to the bank. In the process of the crash and crawling out of the truck I pulled a hamstring and took a hit to my forehead, knee, and rib cage. My biggest concern was the hamstring. At my age thinking it was not going to heal fast. The diagnosis was a tear in the Biceps Femoris (Hamstring tear). Since I have been testing your magic Jar of Energy (bioenergy generator 100) for my back issue, maybe it will help. Other than a wrap and Aleve I have not been able to do much but met this morning with the therapist and he wants to know what I have been doing? He thinks my recovery has been “remarkable”. I am not moving fast but the pain is minimal when I walk and I am not ready to run or work out but I can't help but think the “Jar of Energy” had something to do with it. I still sleep better, wake up in the morning refreshed, and have less arthritis inflammation in my hands. The patient utilized a 1 kg bioenergy generator 100.
Example 3. Reduced pain due to arthritis. An old gentleman had a history of arthritis on his two hands for years and had the pain day and night. He placed the portable bioenergy generator 100 on his nightstand during the night. Then, he had less arthritis inflammation in his hands. The patient utilized a 1 kg bioenergy generator 100.
Example 4. Promoting health and skin looking. A senior lady had fatigue symptoms for a while. She placed the portable bioenergy generator 100 next to her nightstand during the night. One week late, she observed that her skin felt smoother, and she could sleep a lot better. Her energy level becomes high and she could do a lot more work than before. The patient utilized a 1 kg bioenergy generator 100.
Example 5. Treat high blood pressure. An overweight gentleman had high blood pressure for years and needed multiple medicines to control his blood pressure. He placed the bioenergy generator 100 next to him during the night's sleep. One week later, he went to his cardiologist to do the normal follow-up visit. The nurse and the doctor repeatedly measured his blood pressure. All the measurements were similar: his blood pressure readings were around 120/80. His doctor was wondering what brought his blood pressure to such a healthy level. He told his doctor about sleeping with a bioenergy generator 100. His doctor planned to verify this case. The patient utilized a 1 kg bioenergy generator 100.
Example 6. Promoting libido. A gentleman over 60 years of age used the portable bioenergy generator 100 for five nights. After the second night, he felt that the morning erection of his reproductive organ became strong. After 5 nights, he happily reported that his libido level was almost the same as 10 years ago. The patient utilized a 1 kg bioenergy generator 100.
Example 7. Repair Cognitive Impairment. An old lady over 80 years of age suffered from Dementia for several years. She was scored to have severe cognitive impairment (score 25) two months ago. After she used the bioenergy generator 100 generator for 48 days, her dementia total score was reduced to the level of mild cognitive impairment (score 14). She participated in her family activities more often and could remember what was happening in the past. Her energy level was also significantly improved. The patient utilized a 1 kg bioenergy generator 100.
Example 8. Reduced back pain. A senior female patient with a history of back pain for more than 10 years. After she used the bioenergy generator 100 for three nights, her back pain was significantly reduced. She could do more work with less impact from her old back pain. The patient utilized a 1 kg bioenergy generator 100.
Example 9. Reduced menstruation pain. A mid-aged lady has a history of menstruation pain. After she used the bioenergy generator 100 during her 4-day period, her pain level was reduced to almost non-noticeable. She did not have that monthly suffering this time. The patient utilized a 1 kg bioenergy generator 100.
Example 10. Reduced symptoms due to prostate enlargement. A 78-year old male patient had a prostate enlargement for years. He needed to go to the bathroom day and night often in an urgency. After he used the bioenergy generator 100 during the 5-day-night test-use period, he reduced his frequency of bathroom visits by more than 50%, and also reduced the urgency to go to the bathroom. The patient utilized a 1 kg bioenergy generator 100.
Example 11. Promoting hair regrowth. A 64-year old male lost his hair on the top part of his head for more than 5 years. After he used the bioenergy generator 100 during the 25-day-night test-use period, his hair reappeared on top of his head. His hair on his front head also seemed to become dark as compared to one month ago-many white hairs then. The patient utilized a 1 kg bioenergy generator 100.
Example 12. Promoting calmness. A 27 years old young man has a long history of attention deficit hyperactivity disorder (ADHD). After he placed the portable bioenergy generator 100 on his chest, he felt an immediate effect of calmness. He used the bioenergy generator 100 during his sleep time for 5 nights. He could sleep much better every night. His energy level was much higher than before, and his overall behavior during the working hours was much like a more mature man. The patient utilized a 1 kg bioenergy generator 100.
Example 13. Increasing Life Force A group of volunteers test-used the newly invented portable bioenergy sphere generator, and their Life Force Units were measured weekly. The changes for each patient are as follows: A F: 13K to 16 K after two weeks of sleeping with 1 Tesla BioHealer; B D. 14K to 17 K after three weeks of sleeping with 1 Tesla BioHealer; I D. 14K to 17 K after three weeks of sleeping with 1 Tesla BioHealer; C M. 14K to 21 K after five weeks of using 1 Tesla BioHealer; J L. 19K to 22 K after 3-nights of using 4 Tesla BioHealers. All volunteers did not have any side effects. These patients utilized a 1 kg bioenergy generator 100.
Example 14. Reduced the suffering from a brain tumor. A middle-aged woman had a non-operatable brain tumor and suffered the pain and other symptoms daily. After she placed the portable bioenergy generator 100 nearby her body every night for five nights, she felt significant relief from these symptoms. The patient utilized a 1 kg bioenergy generator 100.
Example 15. Help the stroke patient's recovery. A 65 years old woman was paralyzed after a stroke. She placed two portable bioenergy generator 100 in her bed all the time. After two weeks, her paralyzed arm and leg showed a sign of increased movement. She could sleep better and felt energized. She was continually using the devices to help her towards a full recovery. The patient utilized a 1 kg bioenergy generator 100.
Example 16. Improved the symptom of a dog with leg arthritis. The dog owner observed her aged dog could not move much in the last few months. The veterinary medical doctor examined her dog that the arthritis of the legs was the reason for the dog's less movement. After she placed the portable bioenergy generator 100 in her dog's bed for 3 days-nights, she saw her dog could move much easier than before. She continually observed for another 10 days. Her dog almost acted like a normal dog with the freedom of running around. The patient utilized a 1 kg bioenergy generator 100.

Referring to Examples 17-26 below, all biological bodies need to have a normal life force energy. Our life force energy comes from foods we eat, come from oxygen in the air we inhale, and come from water we drink, and also come from the environment. After these foods and drink were increased their life force, their value and benefits increased.
Example 17. Increased the life force of water. It was tested that the bottled water can be charged with the life force by using the newly invented bioenergy generator 100. Six bottles of water were placed on the top of the bioenergy generator 100, and the other six bottles of water were placed in a normal room without any additional bioenergy source, both for 24 hours. The life force unit was measured by using the Bovis bio-scaler. These bottles of water placed on the top of the bioenergy generator 100 were measured for their life force to be 18,000 units. These bottles of water placed in the normal room were measured for their life force to be 13,000 units. This test was conducted utilizing a 12 kg bioenergy generator 100.
Example 18. Increased the life force of an alcohol beverage. It was tested that the rice wine can be charged with the life force by using the newly invented bioenergy generator 100. Three bottles of rice wine were placed on the top of the bioenergy generator 100, and the other three bottles of rice wine were placed in a normal room without any additional bioenergy source, both for 24 hours. The life force unit was measured by using the Bovis bio-scaler. These bottles of rice wine placed on the top of the bioenergy generator 100 were measured for their life force to be 19,000 units. These bottles of rice-wine placed in the normal room were measured for their life force to be 14,000 units. This test was conducted utilizing a 12 kg bioenergy generator 100.
Example 19. Increased the life force of all-purpose flour. It was tested that all-purpose flour can be charged with the life force by using the bioenergy generator 100. Two boxes of all-purpose flour were placed on the top of the bioenergy generator 100, and the other two boxes were placed in a normal room without any additional bioenergy source, both for 24 hours. The life force unit was measured by using the Bovis bio-scaler. These boxes placed on the top of the bioenergy generator 100 were measured for their life force to be 21,000 units. These boxes of all-purpose flour placed in the normal room were measured for their life force to be 14,000 units. This test was conducted utilizing a 12 kg bioenergy generator 100.
Example 20. Increased life force unit of multiple vitamin tablets. It was tested that the bottled multiple vitamins and minerals can be charged with the life force by using the newly invented bioenergy generator 100. Six bottles of multiple vitamins and minerals were placed on the top of the bioenergy generator 100, and the other six bottles of multiple vitamins and minerals were placed in a normal room without any bioenergy source, both for 24 hours. The life force unit was measured by using the Bovis bio-scaler. These bottles placed on the top of the bioenergy generator 100 were measured for their life force to be 20,000 units as compared to 13,000 units 24 hours before the test. These bottles of multiple vitamins and minerals placed in the normal room were measured for their life force to be 13,000 units before and after the 24 hour test time. This test was conducted utilizing a 12 kg bioenergy generator 100.
Example 21. Increased the alcohol content of the bottled wine. It was tested that the newly invented bioenergy generator 100 can increase the ethanol content. Six bottles of wine made from the fermented strawberry were placed on the top of the bioenergy generator 100, and the other six bottles of the strawberry wine were placed in a normal room without any additional bioenergy source, both for 48 hours. The life force unit was measured by using the Bovis bio-scaler. These bottles placed on the top of the bioenergy generator 100 were measured for their life force to be 19,000 units. These bottles of wines placed in the normal room were measured for their life force to be 13,000 units. The two taste panelists also tested that the bottles energized with the bioenergy generator 100 tested much stronger than the control. The ethanol meter showed that the two readings were 79.88 proof and 79.74 proof, respectively, for the energized and the control. This test was conducted utilizing a 12 kg bioenergy generator 100.
Example 22. Increased speed of fermentation in wine production. After 24 hours of adding all materials and water into the two wine fermentation tanks, nothing happened. The bioenergy generator 100 was then placed on the top of the tank at 8:30 pm. The next morning at 8:30 am, that tank had many air bubbles. The other tank without placing the bioenergy generator 100 was still quiet, without observable fermentation. Therefore, it showed clearly that the bioenergy generator 100 helped the speed of the fermentation. This test was conducted utilizing a 12 kg bioenergy generator 100.
Example 23. Increased speed of fermentation in wine production. After 24 hours of adding all materials and water into the two fermentation tanks, nothing happened. The bioenergy generator 100 was placed on the top of the tank at 8:30 pm. The next morning at 8:30 am, that tank had many air bubbles. The other tank without placing the bioenergy generator 100 was still quiet, without observable fermentation. Therefore, it showed clearly that the bioenergy generator 100 helped the speed of the fermentation. This test was conducted utilizing a 12 kg bioenergy generator 100.
Example 24. Increased blooms on time of rose flower. Twelve rose flowers in the test group were provided with a bioenergy generator 100 for 20 minutes each day, 10 minutes was done at 8:00 am, and the other 10 minutes was done at 8:00 pm. The control twelve rose flowers were placed at the normal room temperature. The rose flower energized with a bioenergy generator 100 had nice blooms on for 14 days, while the control had 9 days. This test was conducted utilizing a 1 kg bioenergy generator 100.
Example 25. Keep fruits and vegetables fresher longer. It is desirable to increase the length of the freshness of vegetables and fruits in the refrigerator. Blueberries, strawberries, spinach, red grape, white grape, green pepper, banana, were easy to lose their freshness. For the Test-Treatment, one set of these was placed in the refrigerator along with the bioenergy generator 100 for 5 days. For the Control-Treatment, the other set of the same fruits and vegetables were placed in the refrigerator without the bioenergy generator 100 for 5 days. At the end of the 5-day test, these fruits and vegetables were measured for their life force and visually observed their freshness. For the set kept alone with bioenergy generator 100, their life force measurements were either increased, or not reduced, or reduced less than that in the Control. Below was the table to record these measurements. This test was conducted utilizing a 1 kg bioenergy generator 100.

### Life Force Units of the Fruits and Vegetables

| | Control Set | | Treatment Set | |
|---|---|---|---|---|
| | Before | After 5 Day | Before | After 5 Day |
| Red Grape | 14K | 13K | 14K | 16K |
| White Grape | 14K | 13K | 14K | 16K |
| Spinach | 15K | 13K | 15K | 14K |
| Strawberry | 16K | 12K | 16K | 14K |
| Blueberry | 16K | 12K | 16K | 15K |
| Green Pepper | 16K | 14K | 16K | 15K |
| Banana | 14K | 13K | 14K | 15K |
| Total Unit | 105 | 90 | 105 | 105 |

A panel of three people observed the freshness of these fruits and vegetables. In the Control set, red grape, white grape, spinach, strawberry, blueberry, and banana significantly lost their freshness. The counter fruits and vegetables in the Treatment set were still fresh. The bioenergy generator 100 can be used to keep the freshness of fruits and vegetables.
Example 26. Increased speed of regrowth of a Money-tree stem. It was a common technique to breed the Money tree plant through stem cuttings and the best time to do this is in the summer. The test was done in July 2020 by cut the stem 15 cm long and place them in the soil immediately. Four stems were made. Two were placed next to the bioenergy generator 100 for 30 minutes each day for 7 days (15 minutes at 8:00 am and 15 minutes at 8:00 pm). The other two were placed in the normal room, also for 7 days. The two stems energized with the bioenergy generator 100 had new leaves 2 days faster than the control stems. This test was conducted utilizing a 1 kg bioenergy generator 100.
Example 27. Improved Brain Clarity for a Patient with Lyme Disease. A 54-year old female had signs of post-treatment Lyme disease. She often had fatigue, pain foggy, hard to sleep, and a certain level of depression. After she used the bioenergy generator 100 for 5 nights, she slept much better, and her brain function seems improved a lot. She felt that her brain foggy was gone. This test was conducted utilizing a 1 kg bioenergy generator 100.
Example 28. Relived Chronic Reginal Pain Syndrome. The patient was a 52-year-old female who was diagnosed with Chronic Reginal Pain Syndrome (CRPS). CRPS was a mysterious chronic pain disorder usually affecting a single limb. This patient used a bioenergy generator 100 for 7 nights and her pain level was significantly reduced. This test was conducted utilizing a 1 kg bioenergy generator 100.
Example 29. Helped the Patient with Anoxic Brain Injury. The female patient suffered an anoxic brain injury due to acute myocardial infarction (heart attack). Her daughter stated that since she was placing the bioenergy generator 100 1 foot next to the patient's head, the patient was sleeping more and the energy level was higher than before. This test was conducted utilizing a 12 kg bioenergy generator 100.
Example 30. Helped the Patient with Chronic Back Pain. A senior female patient has suffered chronic back pain for the last 15 years The newly made pad (containing the mixture of 1 kg) with life force (21K Units) energy and infrared wave generated from tourmaline powder. After one week of sleeping on the pad, the patient reported that her back pain was significantly reduced. She can help the family to do a lot more housework.
Example 31. Helped the Patient with Depression. A 49-year old female had chronic back pain then progressed to a herniation at her low back. She progressed quickly to severe depression. After using the bioenergy generator 100 for 7 days-nights, the patient reported that her mood was improved significantly and she was actively collaborating with her physician to treat her injured back. This test was conducted utilizing a 1 kg bioenergy generator 100.
Example 32. Maintain a High Energy Level. A 26-year old man was not a professional athlete but he likes to play tennis and run a marathon. In the past, he felt very tired at the last ⅕ of the 5-mile marathon. The man was sleeping on the newly made pad containing the 1 kg mixture with force (21K Units) energy and far-infrared wave. After two-week of sleeping on the pad, the young gentleman was running another 5-mile marathon. He had much higher energy during the entire run and his over-performance was significantly improved. He also played tennis with a much-improved energy level.
Example 33. Helped the Diabetes Patient. A 53-year old female had type 2 diabetes for more than 5 years. She must self-inject insulin daily. After using the sphere generator made pad (containing the mixture of 1 kg) with life force (21K Units) for 5 days-nights, the patient reported that her energy level was improved significantly, and she could eat well. After meeting with her physician, the doctor decided to reduce her insulin dose by half.
Example 34. Helped the Cancer Survivor. A 71-year old female with breast cancer had surgery to remove her cancer and then had 4 cycles of chemotherapy. She often felt very tired every day. After using the bioenergy generator 100 for 7 days-nights, the patient reported that she could sleep better, her energy level was much higher, and her appetite was also improved. This test was conducted utilizing a 1 kg bioenergy generator 100.
Example 35. Helped the Musician. A 62-year old female music teacher had her hand-finger arthritis for many years. In the past, when she played an instrument, she just took a pain killer. She played the bioenergy generator 100 on her nightstand for 4 nights, then she felt that her arthritis symptoms were reduced and she slept better. She continually slept along with the bioenergy generator 100 for 14 nights, and her symptoms of arthritis were almost gone. This test was conducted utilizing a 1 kg bioenergy generator 100.
Example 36. Help to Energize the Spouse. A 44-year old female patient placed the bioenergy generator 100 under her pillow to sleep for three nights. She was sleeping better and gained more energy. And her husband received very similar benefits. The effective space for the bioenergy generator 100 is more than 2 cubic meters so her husband received effective bioenergy, although the source of the energy was directly under the pillow of the patient. This test was conducted utilizing a 1 kg bioenergy generator 100.
Example 37. Help to Energize the Pet. A 45-year old female used the bioenergy generator 100 for two weeks and reported that “My personal experience has been a great one. . . . I feel more renewed upon waking as well as with pain relief. . . . It's funny. . . . When my dog is napping I put it by her. . . . I see much more vitality in her already . . . ” This test was conducted utilizing a 1 kg bioenergy generator 100.
Example 38. Help to Speed Up Qigong Practice. A 46-year old man practiced qigong for the last 10 years with normal slow progress. When he placed the bioenergy generator 100 next to him when he practices qigong, his qigong energy increased much faster than before. This test was conducted utilizing a 12 kg bioenergy generator 100.
Example 39. Help to Increase the Patient's Chi (Qi). An acupuncturest has been practicing TCM in the State of New York for many years. She likes to use medical devices to increase the chi of her patients. She has provided the portable bioenergy generator 100s to her patients at the end of the office visit. She did follow up and observed that the additional treatment with chi-supplier helped the patient have a quick recovery of the energy level particularly for these patients who had chemotherapy or radiotherapy after the cancer surgery. This test was conducted utilizing a 1 kg bioenergy generator 100.
Example 40. Help to Increase Freedom of Life. A small business owner who managed her senior center in the State of Wisconsin for the last 20 years. The major daily works for the center was to help the seniors to have a normal daily life. The manager provided the portable bioenergy generator 100 to one member who needed the most care daily. One week thereafter, that senior gained a lot of strength and could conduct most daily living activities, such as feeding, drinking. The small business owner was very happy about using the new device. This test was conducted utilizing a 1 kg bioenergy generator 100.
Example 41. Using two Tesla BioHealers (one type of bioenergy generator of the present disclosure) to improve chronic obstructive pulmonary disease (COPD). J. C.'s husband has COPD/Emphysema. J. C. used two Tesla BioHealers to help her husband. Using two J. C. reported that her husband's COPD status significantly improved, and his energy level improved within a month. After three months, he no longer needed his nebulizer. He has occasional flare-ups, but overall, his improvement has been remarkable to his family members. Now he breathes easily. Their son was so impressed with his father's progress that he bought two BioHealers for himself. The first time he used them, he felt such strong vibrations that he had to move them further away. He has since obtained two BioHealers for his fiancée as a big gift. Everyone in J. C.'s family was curious about what's in the BioHealers, and J. C. referred them to the Teslabiohealing.com website for information. J. C. thought it was important to allow everyone to make their own health decisions. Freedom in health choices is paramount.
Example 42. Using two Tesla BioHealers to improve COPD and sleep. C. S. had COPD that had progressed to the point of causing severe pain due to ballooning lungs. After she used Tesla BioHealers for a few weeks, she could go for pain-free walks and no longer cough as much, allowing her to sleep 8 to 9 hours a night. She had not slept that well in years. She was very thankful for the new product.
Example 43. Using two Tesla BioHealers to improve COPD, asthma, and sleep. D. M. used two BioHealers for 4 weeks. He had a fractured hip, fused vertebrae, shoulder damage, and other health issues. He was on 25 medications, including opioids, but managed to get off them despite his doctor being a drug pusher. Since using the BioHealers, his pain has calmed down significantly. His asthma, COPD/emphysema, and breathing all improved. He wanted to use a much powerful product towards being drug-free. Tesla BioHealers have reduced his pain levels drastically, and his breathing has improved. He can now walk longer with less discomfort. He was looking forward to seeing more positive results.
Example 44. Using two Tesla BioHealers to improve COPD, Lung Cancer, energy and sleep. C. C. obtained two BioHealers for her husband, who had severe lethargy and trouble sleeping due to lung cancer and COPD/emphysema. Since using the BioHealers, he has been more energized and ready to go to work every morning, a significant improvement from before.
Example 45. Using two Tesla BioHealers to improve COPD. M. T. initially bought two Tesla BioHealers and a noticeable improvement in breathing was quickly experienced after one week of use. He had COPD and an infection in his mouth implants. While he still had both conditions after one week of use, his COPD symptoms have improved.
Example 46. Using two Tesla BioHealers to improve congestive heart failure, COPD and asthma. P. P. obtained two Tesla BioHealers and noticed an immediate difference. Her COPD and asthma symptoms improved within a few days, and she felt better overall. Despite severe intercostal muscle pain, she improved much faster than expected. Her congestive heart failure is now well-controlled, and she felt stronger each day. Per P. P.'s experience, Tesla BioHealers have provided amazing results, with slow but steady progress. She could go grocery shopping without exhaustion and walk around their circular drive. She had been using the Kryon method for over a decade, and the healing process has accelerated with these canisters.
Example 47. Using two Tesla BioHealers to improve congestive heart failure, COPD, asthma, and pain. P. P used two BioHealers at the beginning of the trial treatment. She noticed a difference immediately on the first day of use. After setting the two BioHealers on a table and leaving the room briefly, she felt a strong energy surge upon returning. She was 75 and had multiple health issues, including COPD and acute asthma. Within a few days, she was breathing easier and generally feeling better. She obtained two more BioHealers because she could already see the benefits. Despite contracting intercostal muscle pain on both sides of my rib cage, the pain improved much faster than expected. By the end of the third week, she felt significantly better. Her congestive heart failure and asthma were well-controlled. Two months ago, she was not sure how long she would live, but now she felt stronger each day. Tesla BioHealers have provided her with amazing results, with slow but steady progress. She recommends starting with two BioHealers as they help. She can now go grocery shopping without exhaustion and walk around her circular drive. This was amazing progress for her. She had been using the Kryon method for over a decade, and the healing process has accelerated with these canisters.
Example 48. Using one Tesla BioHealer to improve hypertension and COPD. D. T. had COPD and pneumonia about five and a half years ago. He reported that he used one Tesla BioHealer for two months, and it seems to be doing what it is supposed to. Before using the Tesla BioHealer, he was doing breathing treatments with ipratropium bromide about six times a day. Now, he used the same medicine no more than twice a day, and his breathing was much better. Additionally, his blood pressure was slowly returning to normal. Initially, he was skeptical, and it didn't seem to be doing much. But gradually, he started seeing results. His goal was to get rid of his wheelchair. He can now walk about 150 to 200 feet, compared to 30 to 50 feet before. He also started using an exercise bike to strengthen his legs. Now he quit all his pharmaceutical drugs, which included medication for blood pressure, blood sugar, and cholesterol, as he no longer needed any of them. He knows Tesla BioHealer was working for him.
Example 49. Using two Tesla BioHealers to improve diabetes, COPD, heart disease. G. W. had diabetes, COPD, heart disease, and needed surgeries for carpal tunnel and a knee replacement. Since using the BioHealers, she had reduced her diabetes medication by 40% and did not need to use her Albuterol inhaler for a while. Her pain level has decreased significantly. She was looking forward to the rollout of med beds and hoped to visit one.
Example 50. Using one Tesla BioHealers to improve Myasthenia Gravis. J. C. had Myasthenia Gravis, a rare autoimmune disorder. She had significant weakness in her muscles. After she placed one Tesla BioHealer next to her body during the night sleeping, she significantly improved her symptoms of Myasthenia Gravis. She stated that was a true believer.
Example 51. Using one Tesla BioHealers to improve Myasthenia Gravis and rheumatoid arthritis. M. L. had rheumatoid arthritis and myasthenia gravis. After she used one Tesla BioHealer for 56 days (7 weeks), she got another one. When she started using the Tesla BioHealers, she took pictures of her hands. She was happily surprised that the good result was almost unbelievable. Her family members and friends were shocked. Her neurologist and rheumatologist could not believe it. She gave them the information about Tesla BioHealers the doctors were going to do research on the product. M. L. has 4 sisters and all of them were using BioHealers. She sincerely wanted to send pictures of her hands.
Example 52. Using two Tesla BioHealers to improve hypertension and Long COVID. J. J. had hypertension for years and had Covid a month earlier, before coming to Tesla Medbed Center for the overnight experience. The first thing she noticed was the tremendous detoxing she believed was trying to get rid of anything left by the Covid viruses. When she returned home with two Tesla BioHealers within two weeks, she noticed her blood pressure and pulse had improved within normal limits. Within a month she noticed that her hair was also growing faster (a lot of her hair fell out due to Covid). She was beginning to get more of a restful sleep every night and slept for longer hours. It seems to be helping her blood pressure and calming down her nervous body. She kept the devices with her all the time and was very happy with them.
Example 53. Using two Tesla BioHealers to improve hypertension and neck pain. J. J. had neck pain for a long time but was gone after using two Tesla BioHealers for two weeks. Her right hip and leg suddenly were giving her trouble but now gone after using two Tesla BioHealers. She also had pulmonary fibrosis which she did not experience if Tesla BioHealers could help. Her high blood pressure had not much improved for a while but now it gradually improved, and it was gone. She would recommend Tesla BioHealers to anyone who has her health problems.
Example 54. Using two Tesla BioHealers to improve energy and sexual drive using two Tesla BioHealers. A Navy veteran reports that his friends think he's lost his sexual drive, but he had gotten it back after he used Tesla BioHealers. He expresses skepticism about how a metal can of sand-mix could make a difference, but the product indeed worked for him. He felt ten years younger, found himself taking stairs two at a time, and could perform heavy yard work for hours with less fatigue and faster recovery. He was sleeping with the devices and experienced deep, vivid sleep and woke up feeling tingling and excited. Despite having tinnitus, which has not disappeared after seven weeks with two BioHealers and three nights with a MedBed, he remains optimistic as it seems reduced. He believed that the devices have anti-aging effects, extending his life and improving his life quality. He recommends BioHealers for most people and MedBeds for those with more serious issues.
Example 55. Using two Tesla BioHealers to improve energy and sexual performance. J. K. expresses amazement, stating that he felt 30 years younger and loved the life force provided by the products. At 75, he claimed to perform like a 50-year-old, with better sleep, manageable blood sugar levels, and improved sexual performance.
Example 56. Using two Tesla BioHealers to improve blood pressure, energy and sleep. R. B. shared her positive experience with the BioHealer, which she initially bought for her husband who had trouble sleeping deeply. She used it herself first and experienced deep, refreshing sleep with vivid dreams. Her husband also started sleeping better and feeling reenergized after a few weeks of use. Both she and her husband now have more energy, feel calmer, and are in better moods. Rose notes a significant drop in her blood pressure, from 130/80 to 113-117/78.
Example 57. Using two Tesla BioHealers to improve cognitive capacity and motor functions. S. I. gave Tesla BioHealers high marks, detailing her husband's progress in daily tasks after using two Tesla BioHealers. Her husband was suffering from TBI and Dementia Pugilistica in the past. Now he sits up straight, eats neatly, and helps dress himself. After starting two Tesla BioHealers, they were adding two MedBed generators. His cognitive and motor functions improved significantly, allowing him to engage with family more naturally and remember his wife.
Example 58. Using two Tesla BioHealers to improve energy, better sleep, and reduced back pain with one Tesla BioHealer and visiting the MedBed Center. K. L. reported her positive experience with the Friends and Family package at a MedBed Center. She notes reduced back pain, increased energy, better sleep, and improved skin clarity. Her dogs also show increased vitality around the BioHealer. She appreciates the clean and comfortable environment of the center and the helpful staff.
Example 59. Using two Tesla BioHealers to have younger-looking skin. K. K. reports improvements in eyesight and younger-looking skin after using the BioHealer for 4 weeks. She placed the devices next to her head during the night and kept them nearby her during the daytime. She described these as the best benefits received.
Example 60. Using two Tesla BioHealers to increase energy and younger looking. J. N. states that herself and husband have more energy than ever, with cellular activity back to optimal levels. They feel balanced and are living younger for longer, as confirmed by the lab cellular testing.
Example 61. Using two MedBed generators to significantly improve sleep, pain reduction, energy levels, and a more youthful appearance. R. W. stated that his mother's experience with Tesla BioHealing products was very satisfactory. In the past, she was suffering from crippling health issues, and she saw minimal improvement with other therapies. After using two MedBed Generators for two weeks, she experienced significant improvements in sleep, pain reduction, energy levels, and a more youthful appearance. She can now engage in activities she loves with less dependence on pain medication.
Example 62. Using one Tesla BioHealer to significantly increase energy, better blood sugar and better wellbeing than before. J. M., 75-Year-Old Retired Colonel, reported that he was feeling younger and having a positive outlook after using Tesla BioHealer for two weeks. He experiences an energy enhancement similar to what he felt 30 years ago, better blood sugar management, and reduced feelings of loneliness. As a widower, he feels more comfortable and grateful for his situation, noting a significant improvement in his overall well-being.
Example 63. Using four Tesla BioHealer to have more clarity and focus, better hand movement. A U reported that her mother had a lot of improvements after her mother used 4 Tesla BioHealers for more than 8 hours a day. After two weeks, they were thrilled with her mother's progress. Here's what she has noticed: (1) More Clarity and Focus: She couldn't concentrate at work since the stroke, but now she can. (2) Improved Hand Movement: When she held the device in her left hand (the stroke-affected side) all night, her hand was able to move more the next morning. (3) Playing with Her Dog: She played with her dog on the floor, something she hasn't done since the stroke. (4) Improved Smile: The left side of her mouth is less droopy, enhancing her smile. (5) Better Arm Stretch: Her left arm can stretch further now. She was able to close the trunk of her car from the top, which she couldn't do before. (6) Better Sleep: She's now sleeping through the night, getting 5 to 6 hours of uninterrupted sleep. Before, she kept waking up in the middle of the night. (7) Improved Voice: Her voice has improved with less hesitation and slurring. (8) Left Hand Numbness: Her left hand is still numb, especially her thumb and index finger. Reducing numbness and increasing movement is the goal for the next two weeks.
Example 64. Using four Tesla BioHealer to significantly improve depressive episodes and decreased anxiety. D. E. reported that for as long as she can remember, she has struggled with depression and severe anxiety. She had never been able to find a combination of medication that helps without causing severe side effects, so she went years without the assistance of medication. Since she began using the Tesla BioHealers 3 months ago, she has noticed significant improvements in her depressive episodes, increased energy and motivation, and decreased anxiety, coupled with feelings of calmness, centeredness, and mental clarity. She can't thank this company enough for what the product has done for her.
Example 65. Using four Tesla BioHealer to significantly improve mental clarity. J. S. reported that the 4 Tesla BioHealer has ramped up her energy levels and mental clarity-basically everything it said it would do, it has. She obtained 4 of them and used them day and night for two months. Her overall health has been improved significantly.
Example 66. Using one Tesla BioHealer to significantly improve libido, energy, and sleep. S. D. reported that her husband and herself sleep with it every night. She also kept it on her computer desk during the day to ward off the harmful rays. They noticed that they both slept much better and fell asleep more easily. She believed that Tesla BioHealer has also improved their blood circulation. S. D. stated that it might be her imagination, but she felt a soft, warm, relaxing energy from it at night. Her husband doesn't feel that, though-he tends to be a bit of an Eeyore at times. Despite being seniors, they believe the husband's libido and emotional state have noticeably improved. The positive effects of our BioHealer are subtle, but they know it's a beneficial device.
Example 67. Using four Tesla BioHealers to significantly improve libido, energy, and reduce pain. M. J. B. was using the lifeforce energized facility at the Tesla MedBed Center in Tampa, FL for 4 days. As he stated, his first visit was amazing, but the second one was so powerful that it significantly heightened his libido. I spoke to the center Director about his experience, and she confirmed that it is a normal response for some. The energy was wonderfully comforting and helped with some pain I experience. I am new to this type of healing and highly recommend it for many reasons, with the increase in libido being a bonus. The Center is very calm and serene, which I appreciated as it allowed me to relax and heal despite others receiving treatments and tours. He looked forward to his next two sessions with great anticipation. Thank you, the Center Director, for your expertise and confidentiality. You are truly a gift to the Center and to this method of healing.
Example 68. Using four Tesla BioHealers to significantly improve Hashimoto's symptoms, libido, vital energy using Tesla MedBed equipped facility. M. R. reported that although she did not usually write reviews unless an experience was profound or extraordinary, this was both. During my 1-hour treatment using Tesla MedBed equipped facility, it took only 15 minutes for her to feel relaxed and calm, with an increase in vital energy she has not felt in years. She felt fantastic after the session and realized she has not had this much energy since being diagnosed with Hashimoto's in her 20s (She is now 52). That night, she slept like a baby and had vivid dreams for the first time in years. The next day, she felt calm, energized, and clear all day. As a professional leadership coach, podcaster, and author, focusing on her current book project had been challenging. However, within 30 minutes of sitting down to write, she outlined the entire book, something she had been struggling with for a year. Her husband and business partner remarked on how radiant, calm, and articulate I was. Additionally, my eczema, which I've had for years, cleared up in one ear and is improving in the other. The unexpected boost in libido was also appreciated. She cannot wait for my next session.
Example 69. Using Tesla BioHealer to treat chronic arthritis and pet tumors. R. L. reported that after he used two Tesla BioHealers for a few days, he was off his sleeping medicines and now enjoyed great sleep. He reduced his arthritis pain significantly and reduced his medicines for treating chronic arthritis just in the first week of using the BioHealers. He hoped to be off all meds soon. Another amazing thing happened with their 14-year-old 2 dogs. They have been restored to puppy status. One pup had large cancer tumors on her back and head, which have now completely disappeared. Their hair, skin, and eyes look amazing, and they are playing and running again like they haven't in years. Friends and family are stunned by the results. He was very impressed.
Example 70. Using one Tesla BioHealer to treat pet tumors. X. A. reported that he purchased the BioHealer for his 13-year-old German Shepherd named Sydney, who had a cancer tumor on her eyelid. CBD oil could only do so much. Sydney sleeps with the BioHealer at night, and X. A. was happy to say the tumor is almost gone. She also seems to be in less discomfort from her hip dysplasia.
Example 71. Using Tesla BioHealer to increase energy and to treat pet tumors. A. P. & J. D. reported that they wanted to share that since they started using the Tesla BioHealer for adults, A. P.'s hands don't hurt nearly as much as they did before. Their dog had a large fatty tumor on her neck, and it's gotten smaller after one week of using Tesla BioHealer. They said they were not kidding! They have more energy. I think the BioHealers are helping them all, including three dogs. They truly appreciate what they have Tesla BioHealer and can't wait to visit a MedBed Center in person. They look forward to each morning now. They emotionally stated that “Tesla! Where have you been all my life?”
Example 72. Using Tesla BioHealer to treat arthritis and to treat pet's Cushing's disease. C. S. reported that “I felt as her arthritis in her joints has improved, and I am sleeping much better! The real miracle was with her little dog, Chloe. She was diagnosed with Cushing's disease a little over four years ago and was expected to live maybe 10 more years with medication. Without it, she would have only had 2 to 3 years. She had a tumor on her pituitary gland due to the disease and had to undergo expensive bloodwork regularly. The medication made her very nauseous, requiring additional medication. I ordered two Tesla BioHealers and started using them in late October 2021. Chloe sleeps with us, with a BioHealer on each side of the bed. During the day, I place one near her bed where she naps. By early December, her bloodwork showed she no longer had Cushing's disease, and the tumor was gone! My vet had never seen a dog recover from Cushing's disease. Whether it was the Tesla BioHealer or a miracle, we are thrilled to have our healthy puppy back. She is eating, playing, and has gained over a pound.”
Example 73. Using Tesla BioHealer to treat arthritis and to treat pet's cancer. K. T. reported that “Our precious Golden Retriever, Zoey, is still going strong in her 21st year, despite living with cancerous tumors and other challenges. Before using the Tesla BioHealer, she had difficulty getting up and walking. I had to pick her up and massage her hind legs before she could move. By the second day, she stood up on her own and walked to the kitchen without stumbling. We were stunned. She is more vocal, more demanding, and walks a little bit further each day. We've even seen her run a little. She has active dreams, running and barking in her sleep. She is more alert and comfortable, with the BioHealer beside her 95% of the time. We highly recommend the Tesla BioHealer.”
Example 74. Using Tesla BioHealer to treat arthritis and to treat pet's cancer. L. W. reported that “My dog, Oreo, has cancer and is undergoing chemo treatments. Sometimes he barely gets out of bed and looks like he is struggling. Since using the Tesla BioHealer, he is acting like a puppy again, running, jumping, and playing like he used to. His bloodwork and vitals are better than ever, according to the vet, who doesn't know about our BioHealers. I definitely recommend it!”
Example 75. Using Tesla BioHealer to stop growth of cancer. P. A. was a cancer patient who used four Tesla BioHealers for 6 weeks and reported that “I waited to share this feedback until I heard from my oncologist about my latest CT scan. He was encouraged that the growth of my cancer metastases has stopped, except for a few minor increases in my liver metastases. He wants to keep me on the same treatment protocol, which usually has a 30% chance of success. I believe the Tesla BioHealers have helped with my cancer. While I am undergoing chemotherapy, I will experience some severe side effects requiring transfusions, etc., so my ability to function well will be diminished. Thank you.”
Example 76. Using one Tesla Medbed generator (another type of bioenergy generator of the present disclosure) to improve cognition, to stop growth of cancer, and reduce extreme pain. T. B. and her husband experienced many benefits as she stated “We have so much good to say! Initially, we noticed more energy and cognitive improvements, but we weren't sure. Now, three months in, my husband's cancer is gone, and we just canceled the surgery. I have a Medbed generator. I had neuropathy in my feet and legs, extreme pain, and was losing the use of my hands. Now, the pain is gone, and I can use my hands again. There was no solution for this medically. My dog, who sleeps in my room with a Tesla Adult BioHealer, had large tumors all over her back and belly, and after three months, we can see they are shrinking. I am seeing more improvements every day. Praise God. Thank you, Tesla BioHealing!”
Example 77. Using one Tesla Medbed generator to reduce pain, and to stop growth of cancer. X. B. reported that “Over the last four weeks, I have been feeling better, with less back pain, more motivation to do things, standing taller, happier, and in a better mood. Additionally, my nurse practitioner informed me that my CT scan showed no new cancerous growths and that the tumors continue to shrink. I haven't yet met with the doctor to see if they have anything else to add. THANK YOU!”
Example 78. Using two MedBed generators to reduce pain from prostate cancer and reduce the tumor size of lymphedema. R. K.'s father had stage 4 prostate cancer and he was suffering from severe cancer pain. R. K. reported that “We believe without a shadow of a doubt that the Tesla Life Force Generators (MedBed) are the foundation of my Dad's recovery from Stage 4 prostate cancer. At the very least, we know that the pain from his metastasized cancer is gone, if it still exists. We took a bone scan with dye, and we still saw the dark spots where the cancer was, but there was no growth. Since we are using this new technology, our doctors may not be accounting for its effects, so their diagnosis might be incorrect regarding whether the cancer still exists. The only pain he deals with now is from lymphedema, which has significantly reduced after using the Tesla Biohealing devices. We can't imagine being without them.”
Example 79. Using two Tesla BioHealers to reduce tumor size. C. H. reported that “I was diagnosed with invasive ductal breast cancer, 5.7 mm, confirmed on Feb. 24, 2024, with surgery planned for Mar. 17, 2024. Your Tesla BioHealers arrived four days before surgery. According to the pathology report, the tumor had reduced to 4.3 mm, and no chemo or radiation therapy was required. My Saint Bernard, Mandy, had swelling on both elbows the size of tennis balls. She sleeps at the bottom of my bed, where I placed one Bio Healer at the head of the bed and another under the mattress at the foot of the bed. Both swellings were gone in two months. Thank you from the bottom of my heart.”
Example 80. Using two Tesla BioHealers to reduce cancer markers. M. M. reported that “In one month, my cancer marker dropped by 246 points. For the last two years, the number had continued to rise. This is the first significant decrease I have seen. I hope it keeps going down!”
Example 81. Using two Tesla BioHealers to reduce side effects of cancer chemo or radiotherapy. J. C. was a 62-year-old male. He had his prostate removed in 2017. His PSA started rising in mid-2019, and he was referred to his urologist for further treatment. He got a BioHealer when he began his 33 radiation treatments. Although he felt a bit tired initially, he did not notice it much and slept very soundly throughout the treatment process. About halfway through, he noticed a significant improvement in his energy levels. Before using a BioHealer treatment, he needed to take breaks of half an hour to two hours to mow his lawn. Now, he can finish his three-quarter acre lawn, including trimming, in one hour. He has also been able to continue his seven-mile walks and worked every day during treatments. He experienced no side effects, and his cancer doctors and nurses were amazed at how well he was doing, expecting him to eventually crash. His doctor recently gave him good news that he would be finished after 33 radiations. He was able to treat the cancer on his pelvis as well.
Example 82. Using four Tesla BioHealers to reduce side effects of cancer chemo or radiotherapy. S. L. reported that she had cancer quite severely, so she was using four BioHealers. She underwent chemotherapy every three weeks and has had two treatments so far. She had seen several doctors who had given her good vital readings and commented that I look good and remain upbeat despite her illness. Her daughter was surprised that S. L. did not need pain medicines yet. The cancer is in her left lung, bones, and she had a tumor against her vocal cord, discovered in March, which has not grown. She also had a tumor in her back that broke her 4th vertebrae, and she had radiation for a spot on her brain. Her prognosis was not great, but she was relying on her four Tesla BioHealers to prolong her life, prevent the chemo from harming her, and continue replacing her cancer cells with healthy ones. She felt the 4 BioHealers were helping. Her oxygen level is 98%, which she thought was good considering her health condition. Additionally, she heard from a friend with Lyme disease that in one of her groups, someone mentioned the Tesla BioHealers as the most helpful treatment, which gave S. L. more hope and confirmed their effectiveness.
Example 83. Using four Tesla BioHealers to reduce cancer pain. J. E. reported that his mother was a registered nurse and had 6/10 hip pain from breast cancer that spread there and had to use a walker. After using the Tesla BioHealing adult unit for two weeks, her pain gradually reduced to 1/10. She uses it all night next to her affected hip. She has been nearly pain-free and walking without any help or walker for two months, and we pray it continues.
Example 84. Using four Tesla BioHealers to reduce cancer pain. M. K. reported that “This amazing product helped my husband through the worst kind of cancer pain. He endured stage 4 bone cancer without any pain medication. His pain went from a 10 to a 0, and doctors are amazed that he no longer takes his pain meds. It worked so well for him that I bought one for my sister, who suffers from an autoimmune disease. She is now able to walk without pain and no longer experiences flares or breakouts. Am I a believer and a fan? Yes, I am.”
Example 85. Using four Tesla BioHealers to reduce cancer pain. C. R. reported that “I bought four BioHealers after being diagnosed with stage 4 peritoneal cancer, a rare and aggressive mutation from another organ. The first two nights I slept with all four units next to my body and had the best sleep in a long time. After that, my nights returned to what was normal for me. I noticed some improvement with pain here and there. I still sleep with them every night, hoping they are improving my quality of life, even if nothing dramatic is apparent. They are too expensive to give up on. The company's good rating means something.
Example 86. Using four Tesla BioHealers to reduce cancer pain. J. L. reported that “We purchased this two weeks ago for a terminal cancer patient. We are praying it will work fast! Since the first day, sleep has been deeper, and waking up is more refreshing. Kidney and back pain have disappeared, and we feel clearer and calmer.”
Example 87. Using four Tesla BioHealers to reduce cancer pain. J. G. reported that “I have been battling cancer for many years and recently received a secondary cancer diagnosis. A friend highly recommended a stay at a MedBed Center. After the first night, my sinuses cleared, which was such a relief. I feel better and can tell my body is detoxing. My ankle pain has decreased, allowing better rotation, and my abdominal pain is greatly reduced. I had a great stay and experienced these results without any changes to my daily routine except for my stay at the center.”
Example 88. Using four Tesla BioHealers to reduce cancer pain. B. L. reported that “I have been suffering from metastatic prostate cancer for eight years and have endured numerous trials as the disease progressed. In September, my wife researched Tesla BioHealer and spoke with your representative. I've been using BioHealer for nearly two months now and have noticed decreased bone pain, less dizziness, and more energy. My PSA level has dropped 150 points. While I can't call it a miracle cure, it has made my life more comfortable. Thank you.”
Example 89. Using four Tesla BioHealers to reduce cancer pain. M. K. reported that “We purchased six BioHealers: two for my daughter, two for my spouse, and two for myself. We have been using them for over two months. My daughter, who is being treated for metastasized breast cancer, feels they have helped increase her energy and decrease her pain. My spouse and I, who are both over 75 with joint and back pain, have also experienced less pain and increased energy. We are very pleased with the product.”
Example 90. Using four Tesla BioHealers to reduce cancer pain. N. N. reported that “We are LOVING the BioHealing canisters, both large and small! My husband, Jim, has had to take pain medication every four hours for the last three years to alleviate his pain from chordoma cancer. He keeps the small canisters near him in his wheelchair or recliner, and the large canisters are under our bed. Since using the Tesla BioHealing products, he can go almost nine hours without pain medication. As people of prayer, we believe that prayer, the BioHealers, and chemotherapy pills are all working together to bless and heal his body. We wish everyone we know could use these special products. Thank you!”
Example 91. Using four Tesla BioHealers to reduce cancer pain. S. M. reported that “My wife has metastatic breast cancer that caused fractures in two of her bones, resulting in intense and almost constant extreme pain. Her oncologist initially prescribed morphine-grade pharmaceuticals for pain relief, which barely helped. After radiology sessions and purchasing the BioHealer units, she is now no longer using any pain medication and sleeps well at night. She has been prescribed a new pill-only regimen for her cancer, and we are cautiously optimistic about the future. The speed and strength of her recovery have been impressive and unexpected. I am confident she would not have made this much progress so quickly without the help of the BioHealing units.”
Example 92. Using Tesla BioHealers to Treat Uncontrolled Glaucoma. Three patients with long standing history of open angle Glaucoma who were found to have uncontrolled intra-ocular pressures in spite of being treated on maximum medical therapy and having undergone surgical or laser procedures and who subsequent to utilizing the Tesla Biophoton Generators demonstrated a reduction of 3 to 5 mmHg in their intra-ocular pressure readings after 4 weeks of use.
Case 1. A 63-year-old white female with a long-standing history of open angle glaucoma on maximum medical therapy including 3 anti-glaucoma medications and Diamox sequels orally who had undergone a canaloplasty surgical procedure in both eyes one year prior with significant decrease in intra-ocular pressure was found to have progressively increased pressures in both eyes after 6 months post-op. Despite placing the patient on maximum medical therapy, the patient continued to have uncontrolled IOP in the 19 to 20 mmHg range in both eyes when the desired range was determined to be 15 mmHg or less due to her degree of optic nerve head damage. The patient was given a Tesla BioHealing adult unit and instructed to use it for at least 8 hours daily by placing it at 3 feet or less away from her as is the recommendation of the Tesla BioHealing company as per their studies regarding the radius of effective energy dispersion from the unit. After 4 weeks of use, the patient returned for a follow-up visit and the intra-ocular pressure readings were OD 14 mmHg and OS 15 mmHg. The patient was kept on her maximum medical therapy as noted above and will return for follow up in 3 months. Optical Coherence Tomography (OCT), Humphrey's Visual Fields and Visual acuity remained unchanged. However, the patient reported experiencing or feeling that her vision had improved.
Case 2. A 72-year-old Hispanic male patient with long standing history of open angle glaucoma in both eyes and significant optic nerve head damage of 0.8 c/d was found to have uncontrolled IOP's in the 18 to 19 mmHg range on maximum medical therapy including 4 eye drops and having had bilateral SLT (laser) treatments. The preferred IOP for his degree of ONH damage was determined to be 16 mmHg or less. Due to his uncontrolled IOPs and progression of nerve cell layer damage as measured by OCT the patient was advised to consider canaloplasty surgery in both eyes. However, an alternative method of treatment was explored, and he was also provided with a Tesla BioHealing adult unit as patient of Case1 and given the same instructions for usage. After 4 weeks of use, the patient returned for a follow-up visit and the intra-ocular pressure readings were OD 16 mmHg and OS 15 mmHg. The patient was then told to continue use of the Tesla BioHealing unit as the ideal IOP had been achieved in both eyes and any further surgery was postponed. The Optical Coherence Tomography was unchanged.
Case 3. A 75-year-old Hispanic female patient with history of advanced open angle glaucoma in her only visually functional eye was found to have highly uncontrolled IOPs on maximum medical therapy that included 4 eye drops and Diamox sequels. The patient had previously undergone cataract surgery and trabeculectomy 4 years prior. The patient's IOP was found to be uncontrolled in the 21-22 mmHg range for her degree of ONH damage which was 0.75 c/d and progression of central visual loss was noted at the 20/50 level. OCT had shown progression of nerve fiber layer loss. The patient was provided with a Tesla BioHealing adult unit as previous patients in Case 1 and 2 and given the same instructions for usage. After 4 weeks, the patient returned for follow-up visit and her IOP was found to be 17 mmHg which was only 1 mmHg above the ideal pre-determined value but still significantly lower and clinically better for her degree of optic nerve head damage. The OCT was also unchanged. The patient was advised to continue on all her medical therapy and continue the use of the Tesla BioHealing unit as indicated and return for follow-up in 2 months.
Example 93. Using Tesla BioHealers to Reduce very severe and worst possible pain. Inventors conduct post-marketing research by using the standard Likert Pain Scale. Four hundred and seven (407) participated in the marketing research. They had no pain or had varied pain level before using the products provided their answers. All of them used Tesla BioHealing® Products. Among them, 96% used Tesla BioHealer for Adults. Below table summarizes the market research results.

TABLE 3
407 Users of Tesla BioHealing Products Reported
Their Pain before and after 1 Month of Use

| Pain Scores | 0 | 1-2 | 3-4 | 5-6 | 7-8 | 9-10 |
|---|---|---|---|---|---|---|
| Pain Grade | No | Mild | Mod- | Severe | Very | Worst |
| | Pain | Pain | erate | Pain | Severe | Possible |
| | | | Pain | | Pain | Pain |
| Number of Users | 27 | 36 | 131 | 98 | 76 | 39 |
| Starting Pain Score | 0 | 1.9 | 3.6 | 5.5 | 7.6 | 9.6 |
| Reduced Pain Score | 0 | 1.0 | 1.9 | 2.9 | 4.2 | 5.1 |
| after 1 Month | | | | | | |
| Remaining Pain | 0 | 0.8 | 1.7 | 2.6 | 3.4 | 4.5 |
| Score after 1 Month | | | | | | |
| Reduced Pain Level | N/A | N/A | 73 | 71 | 60 | 32 |
| at Least 2 Points | | | | | | |
| Effective Rate of | N/A | N/A | 55.7 | 72.4 | 78.9 | 82.1 |
| Reducing Pain At | | | | | | |
| Least 2 Points (%) | | | | | | |

Among 407 users, 5 used Tesla BioHealer for Children (1%), 11 used Tesla BioHealing® MedBed Generator (3%), and 391 used Tesla BioHealer for Adults (96%). Therefore, Tesla BioHealer for Adults is an effective device to reduce pain.

On the average basis 1 month after using the device:
The patients with the “worst possible pain” reduced to “Moderate.”
The patients with “Very Severe Pain” reduced to “Moderate” pain.
The patients with “Severe Pain” reduced to “mild” pain.
The patients with “Moderate Pain” reduced to “Mild pain.”
The patients with “Mild” Pain reduced to “No pain.”

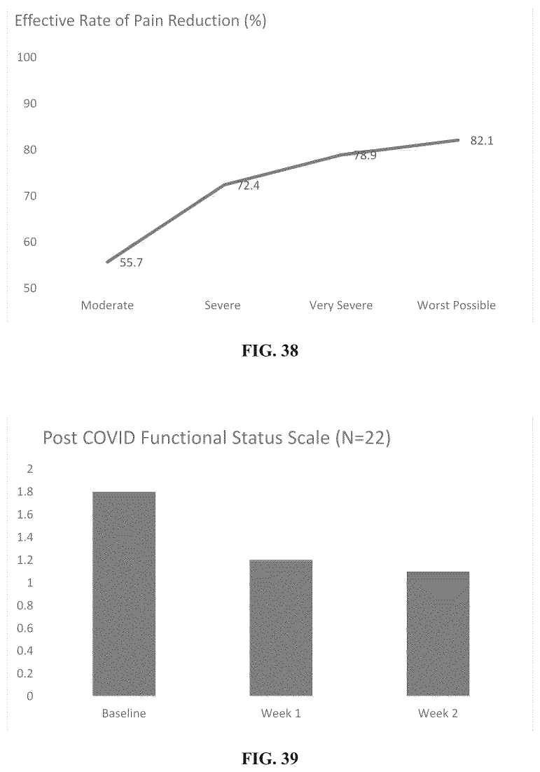

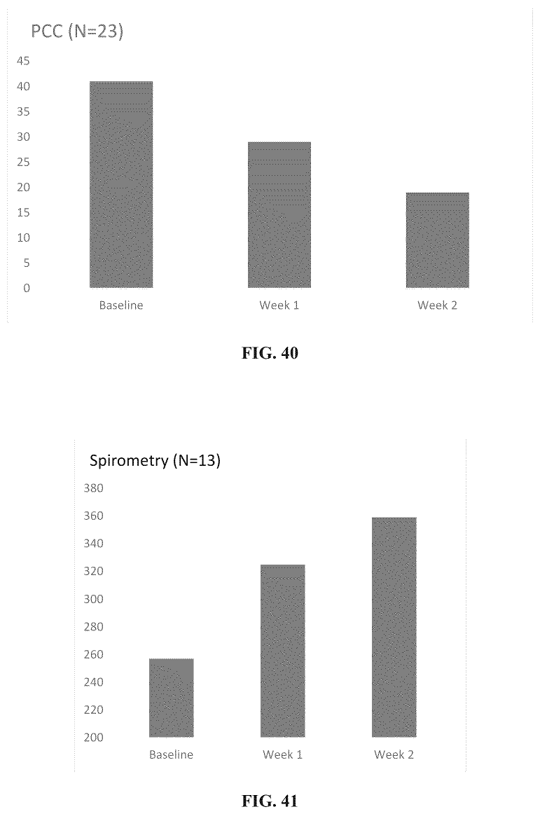

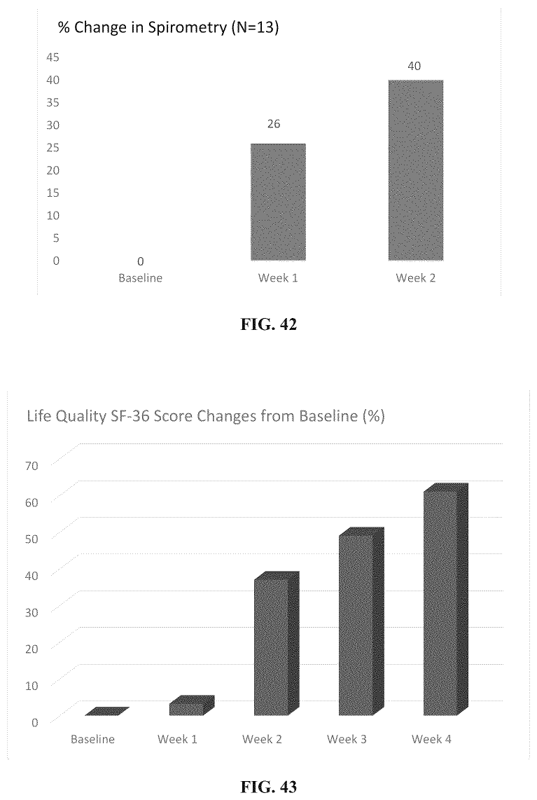

Among those patients with moderate to severe and worst pain (N=344), 289 patients reduced pain at least one level (84%); 235 reduced at least 2 level (68%), and 164 reduced at least 3 levels 48%. It is very important to point out that those who had “Worst Possible Pain” has an 82.1% success rate by reducing pain levels at least for 2 points, and average by 5.1 points. For those with “Very Severe Pain,” the success rate is 78.9%, and the average reduction is 4.2 points. The levels of pain and the treatment effective rates are shown in FIG. 38.
Example 94. Using Tesla BioHealers to Treat Post-COVID. The purpose of our clinical research was to verify if the patients with post COVID-19 conditions (PCC), or Long COVID, can improve breathing capacity quickly by using Testa BioHealing® Biophoton Generators to energize the lung and other organs of the body daily. Below are main outcomes:
   (1) Post-COVID-19 Functional Status Scale (PCFS, a 5-grade version) was significantly improved during the 2-week study period. See FIG. 39. After the 2-week treatment, the 22 patients with Long COVID reduced the severity of many symptoms and the Post-COVID-19 Functional Status was improved weekly.
   (2) The Severity of Post-COVID-19 symptoms was decreased weekly as shown in below figure. See FIG. 40.
   (3) Respiratory capacity was improved. The results of a spirometry tests showed that the FEV1 was increased by 26% as compared to the baseline measurement one week after biophoton therapy, and 40% increase two weeks after biophoton therapy. See FIGS. 41 and 42.

In summary, patients with Long COVID were successfully treated by using biophoton generators as reflected in the decrease in the severity of these symptoms and increase in respiratory capacity. Per CDC, 18 million patients with Long COVID need help. About 18 million US adults have had long COVID: CDC-ABC News (go.com) This is to meet the national need to improve the life quality of 18 million patients with Long COVID.

### Example 95. Treating Patients with Alzheimer's Disease

(1) EEG Reported Behavioral Motor Test in the Patient with Alzheimer's Disease.

The EEG measurements were performed weekly during a 4-week observational study. Below Table 4 recorded certain EEG measurements.

TABLE 4

| | Baseline | Week 2 | Week 4 |
|---|---|---|---|
| Reaction Time Score (ms) | 887 | 816 | 720 |
| Reaction Time Variance | 8 | 3.5 | 3.6 |
| Missed Responses | 20.0% | 1.7% | 0.0% |
| Wrong Responses | 0.0% | 0.0% | 0.0% |

(2) In a preliminary clinical study, patients with Alzheimer's Disease completed the 4-week observational study. Their life quality was improved weekly. See FIG. 43 and FIG. 44. It was observed that the patient's cognitive capacity was improving every week.

In summary, the life quality of all patients with Alzheimer's disease improved significantly after they received the treatment with biophoton generators. Compared with those patients who only received the standard of care (SOC), the newly observed evidence indicated that biophoton generator(s) should be used to treat patients with Alzheimer's disease. The disease progress becomes reversable. The Alzheimer's disease management history has a new chapter.

### Example 96. Improve Cognition and Life Quality of Patients with Dementia

Two seniors with dementia participated in the at-home study and cared for by their adult children who were medical professionals. The two medical professionals completed “The Alzheimer's Questionnaire (AQ©) of their senior parents. The first senior's AQ was 25 at the baseline and changed to 14 after 4 weeks of using the Biophoton Generator. The second senior's AQ was 24 at the baseline and changed to 14 after 4-weeks of using the Biophoton Generator. When the AQ changed to 14, the two seniors previously with severe cognition impairment (AQ >20) were able to live independently.

### (1) Clinical Study Outcomes Using Montreal Cognitive Assessment (MOCA)

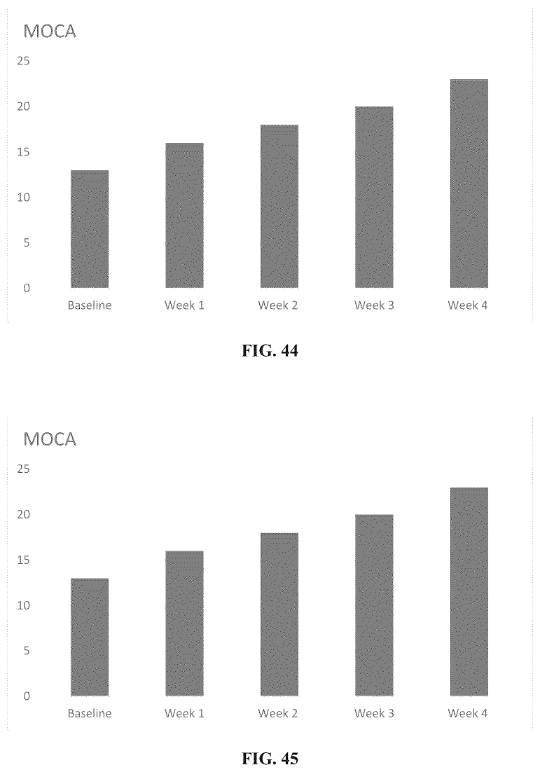

It was observed that the patient's cognitive capacity was improving every week. See FIG. 45.

### (2) Patients or Relative's Reports

Many users bought the wellness products to meet their needs. Some of them were to help family members with dementia. After using the product for a few days or few weeks, they observed a significant improvement in cognition. They sent their feedback to the manufacturer. Below are some of the examples of reporting positive outcomes. No negative feedback on worsening the condition.

A.M. Seattle, Washington reported that “My position as Resident Manager for a senior care facility has allowed me to buy four of your adult healers for the seniors, my employer and myself, and witness the improvements in each of us. (Note: Before giving the Tesla BioHealers to the seniors, I got written permissions from all of their POAs, which was required for our type of facility and all of them were thrilled.)

The first resident, whose POA approved the use, had increased energy and more mental clarity. We noticed she was a lot more relaxed and had more self-awareness. The second resident had less pain, allowing her to tolerate a decrease in her methadone used for a hip injury, and was able to sleep more soundly. The staff noticed the changes in the third resident quicker than she did. Her memory improved as well as her anxiety. Where she would have shut down at the simple 2 choice option, she now can choose logically between 3 options and her anxiety has lessened enough for her to restart her once routine walking outside again. My employer has severe arthritis pain, among other health conditions. She has more energy and can go longer between her pain meds.

For myself, I have nerve damage. I used to have to take methocarbamol to stop excruciating spasms that made me scream, along with the stabbings, pokes, acid-burning sensations, ice-dripping feelings all over from head to bottoms of my feet. Also I, too, have arthritis in my left foot. I started (after following the distance requirement and slowly bringing it closer), resting my left foot on the Tesla adult BioHealer whenever I was taking a break, or while the residents were eating a meal. This caused my left foot to cease the sensation of feeling like the bones were about to crunch or the ankle were about to go into another sprain while going downstairs. Now, I use it between both of my feet to stop the spasms (takes less than a couple of minutes). The Tesla BioHealer works faster than the methocarbamol (which takes 12-15 minutes). I plan on buying more, a second one for each of the residents starting next year. Thank you from all of us.”

W. I. reported that “My father has dementia. He has been using the Tesla BioHealer devices for 6 days now, both he & I have seen & felt many positive benefits. My father's sleep is more consistent & for longer durations throughout the night. He has not wet the bed since he started sleeping with the device directly next to him. He physically got up to urinate on Sunday & went directly back to sleep. Prior to receiving the device, he would wet the bed approximately 3-5 times per week, rarely attempting to get out of bed to urinate, instead depending on the Depends. Due to the newfound balanced sleep patterns, he appears physically (more color in face & hands), mentally & emotionally more aware, alert, positive & motivated throughout the daytime hours. He's been proactively participating in both physical therapy sessions & neurological activities. His speech, reasoning & communication skills appear & sound stronger with directed intent. Just last night he mentioned to me without asking that he believes something is “working” after using the device for 6 days now. I'm thrilled & encouraged by witnessing & experiencing all the positive benefits the Tesla BioHealing device is providing my father & I.”

R. S. reported that “My wife has dementia and I have been taking care of her about 8years. The last 5 years is bed ridden and she cannot sit up or stand or feed herself. She is 82 years old. Two months ago, I purchased the large 2 Tesla healing canasters to put under her bed. Her skin thickness was very thin, and she bruised easily. Now the skin is thicker and firmer and less likely to bruise. She has not spoken a word in over 5 years. She is beginning to speak a few words and even answering questions with a yes or no. She held a cup to drink from a straw for the first time in years. She even sat on the edge of the bed for about 30 seconds without falling over. Seeing what is happening outside is a clue to the good things happening inside, including the brain. I am excited to see what is to come in the next 2 months. She is more alert and aware of her surroundings than before. Thank God to a prayer's being answered and Tesla Bio Healing products.

Rose Reported that “My husband has advanced dementia. Since having the Tesla BioHealing Devices for two weeks, he has been putting on his clothes independently, has more clarity in thoughts and ideas, and has been sleeping better and sound!”

### Example 97. Biophoton co-therapy to enhance tolerance to cancer treatments for optimized outcomes—Case 1

A 48-year-old Hispanic female with a history of advanced breast cancer was treated at Cobre Valley Regional Medical Center in Globe, Arizona. Her treatment regimen included a lumpectomy, radiotherapy, chemotherapy, and a partial nephrectomy to address renal cancer. During her cancer treatments, the patient experienced multiple severe symptoms, including pancytopenia, hip and flank pain, joint pain, severe fatigue, hair loss, weight loss, acute pyelonephritis, recurrent infections, and treatment-related neuropathy.

In October 2021, the patient enrolled in a pilot study program to incorporate biophoton co-therapy into her treatment regimen. After consultation with her family, the patient initiated the use of four Tesla BioHealing® Automatic Biophoton Generators (ABGs), another type of bioenergy generator of the present disclosure. The ABGs were strategically placed around her bed to ensure nightly exposure to biophoton energy during her ongoing cancer therapies.

### Clinical Timeline

November-December 2021: Following the initiation of biophoton therapy, the patient began experiencing notable improvements in side effects, including reduced fatigue and pain. Weekly complete blood count (CBC) tests, conducted to monitor her cancer treatment, showed a return to normal levels by Dec. 23, 2021. These results remained stable throughout the course of her intensive cancer treatments. (Refer to Table 5 below for CBC test results from December 2021 to April 2022.)

April 2022-June 2023: The patient continued her cancer therapies without interruptions, as side effects were significantly minimized. Hair regrowth became evident during this period. Her oncologist, impressed by her progress, inquired about her recovery process. She disclosed her nightly use of four Tesla BioHealing® ABGs. In June 2023, after examining tissue samples under a microscope, a pathologist found no evidence of cancerous cells, indicating a negative result for cancer in the examined area.

June 2024: A routine mammogram and laboratory tests confirmed no evidence of malignancy. The patient was declared cancer-free for one year.

TABLE 5
CBC Test Results from December 2021 to April 2022

| | Normal | Dec. 23, | Dec. 30, | Jan. 7, | Jan. 14, | Jan. 21, | Jan. 28, | Feb. 4, | Feb. 17 | Apr. 5, |
|---|---|---|---|---|---|---|---|---|---|---|
| | Range | 2021 | 2021 | 2022 | 2022 | 2022 | 2022 | 2022 | 2022 | 2022 |
| WBC | 3.4-10.9 | 7.20 | 6,79 | 6.56 | 6.58 | 5.98 | 5.24 | 5.68 | 6.97 | 7.65 |
| ((K/mm3) | | | | | | | | | | |
| RBC (M/mm³) | 3.2-5.5 | 4.03 | 4.04 | 4.31 | 4.35 | 4.38 | 4.21 | 4.32 | 4.54 | 4.43 |
| HGB (g/dl) | 9.7-16.6 | 11.7 | 11.9 | 12.6 | 12.7 | 12.9 | 12.3 | 12.3 | 13.2 | 12.5 |
| HEMATOCRIT | 32.8-46.7 | 36.7 | 36.7 | 38.6 | 39.4 | 39.6 | 38.6 | 39.4 | 39.3 | 38.0 |
| (%) | | | | | | | | | | |
| MCV (fl) | 79.8-100.7 | 91.1 | 90.8 | 90.3 | 90.6 | 90.4 | 91.7 | 91.2 | 86.6 | 85.8 |
| MCH (pg) | 27.1-34.0 | 29.0 | 29.5 | 29.2 | 29.2 | 29.5 | 29.2 | 28.5 | 29.1 | 28.2 |
| MCHC (g/dl) | 31.7-36.0 | 31.9 | 32.4 | 32.4 | 32.2 | 32.6 | 31.9 | 31.2 | 33.6 | 32.9 |
| RDW (%) | 11.9-15.7 | 14.9 | 14.4 | 13.5 | 13.2 | 13.2 | 13.1 | 13.2 | 13.2 | 13.9 |
| PLT.Ct | 128-361 | 294 | 295 | 281 | 247 | 222 | 244 | 239 | 263 | 250 |
| (K/mm³) | | | | | | | | | | |
| MPV (fl) | 8.9-12.2 | 9.6 | 9.5 | 9.1 | 9.2 | 8.8 | 8.9 | 9.1 | 9.2 | 9.1 |
| NEUTR (%) | 41.7-80.1 | 62.0 | ND* | NE | ND | ND | ND | ND | 64.2 | 74.8 |
| LYMPH (%) | 11.5-42.6 | 22.5 | ND | ND | ND | ND | ND | ND | 18.4 | 9.5 |
| MONO (%) | 4.6-12.9 | 5.3 | ND | ND | ND | ND | ND | ND | 9.0 | 7.3 |
| EOSIN (%) | 0.0-5.7 | 9.3 | ND | ND | ND | ND | ND | ND | 6.9 | 7.2 |
| BASOPHIL (%) | 0.0-1.4 | 0.6 | ND | ND | ND | ND | ND | ND | 0.9 | 0.7 |

*ND: Not done,

Outcome:

Biophoton therapy significantly alleviated the side effects of chemotherapy and radiotherapy, enabling the patient to complete her cancer treatments without interruptions and ultimately achieve a cancer-free health status. The patient's quality of life markedly improved, as evidenced by hair regrowth, stable weight, normal daytime energy levels, resolution of anemia, and enhanced immunity. During the cold season, the patient demonstrated a notably higher resistance to common viral infections compared to her family members.

### Example 98. Biophoton Co-Therapy to Enhance Tolerance to Cancer Treatments for Optimized Outcomes—Case 2

A 24-year-old Caucasian female was diagnosed with succinate dehydrogenase (SDH)-deficient gastrointestinal stromal tumor (GIST) in February 2020 by a team of oncologists at Mayo Clinic. She presented with a two-year history of gastrointestinal symptoms, including diarrhea and bloating, prompting an extensive diagnostic workup. Imaging studies revealed massive hepatomegaly with multiple liver lesions, along with a primary gastric mass. Subsequent endoscopy and biopsy confirmed the diagnosis of SDH-deficient GIST.

Oncologic and Genetic Evaluation: Pathology findings showed diffuse positivity for KIT and DOG1, along with loss of SDHB expression. Genetic testing revealed no germline SDH mutations or other pathogenic variants. Tumor genomic sequencing identified several variants of uncertain significance (VUS), including SETD2, BAP1, IKBKE, and BRAF (non-V600E).

Initial Treatment and Progression: The patient was initially treated with VEGF-targeted therapy using sunitinib starting Feb. 28, 2020, which resulted in stable disease after two months. However, by August 2020, imaging revealed progressive enlargement of liver lesions. As a result, her treatment regimen was switched to regorafenib, starting on Sep. 18, 2020, with a dose escalation to 160 mg daily. Imaging in December 2020 indicated stable disease.

On Mar. 17, 2021, while undergoing treatment with regorafenib, the patient was evaluated by her oncologist. Side effects of the cancer therapy included fatigue, anemia, mild headaches, and occasional nausea. Notably, there was no evidence of hand-foot syndrome, mucositis, or other toxicities necessitating dose adjustments.

The patient was clinically diagnosed with five concurrent conditions:

(1) Paraganglioma (HCC), (2) Secondary malignant neoplasm of the liver (HCC), (3) Gastrointestinal stromal tumor of the stomach (HCC), (4) Hypomagnesemia, (5) Paratracheal mass, possibly aorticopulmonary paraganglioma. Other clinical observations included: Constitutional: Positive for fatigue. Cardiovascular: Positive for rapid or fluttering heartbeat. Gastrointestinal: Positive for abdominal pain, cramping, diarrhea, and heartburn. Neurological: Positive for headaches. Psychiatric/Behavioral: Positive for nervousness, anxiety, or feeling on edge within the past two weeks.

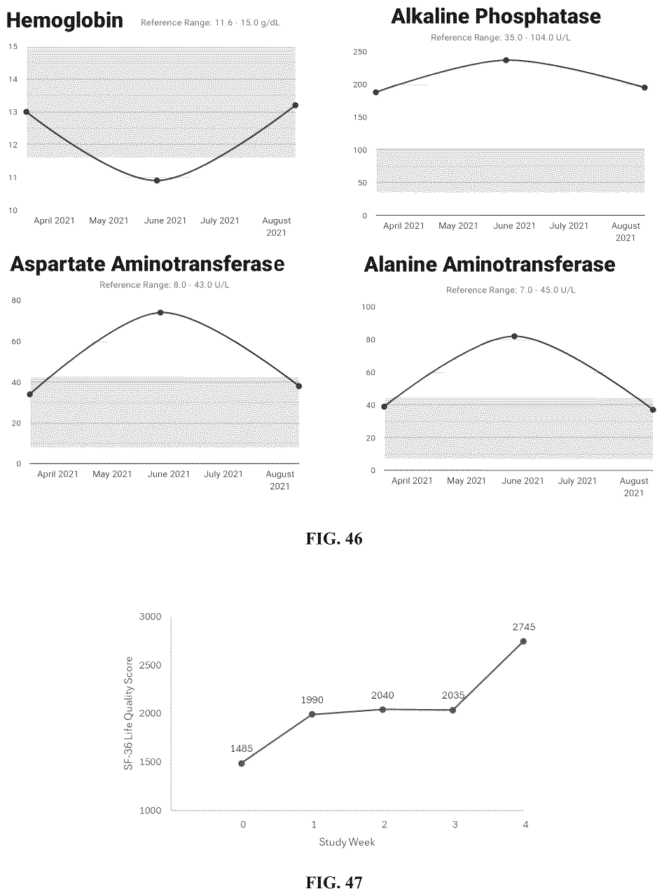

In June 2021, the patient participated in a self-observational study to evaluate the effects of biophoton co-therapy as a complementary treatment. Over an eight-week period, she used four Tesla BioHealers nightly. The therapy resulted in significant weekly improvements in her overall quality of life. She reported reduced fatigue, anxiety, and gastrointestinal symptoms. Additionally, her laboratory results, including hemoglobin levels and liver function markers, normalized during this period, as shown in FIG. 46.

The improvements were attributed to the supportive effects of biophoton co-therapy in enhancing her energy levels and tolerance to ongoing cancer treatments. The therapy also contributed to her ability to delay liver transplant surgery while maintaining her quality of life.

### Example 99. Biophoton Co-Therapy to Enhance Tolerance to Cancer Treatments for Optimized Outcomes—Case 3

The patient, a 62-year-old Caucasian male, had a medical history of prostate cancer removal in 2017. In mid-2019, rising PSA levels indicated recurrent prostate cancer, prompting a referral for follow-up and subsequent treatment. The patient underwent 33 sessions of radiation therapy as prescribed by an oncologist at Johns Hopkins University Medical Center.

In 2021, the patient visited the Tesla BioHealing Office and decided to participate in a pilot observational program. He began using one Tesla BioHealer nightly, incorporating it into his daily routine at the start of his radiation treatments.

### Observations and Patient Experience:

Energy Levels: Before starting radiation therapy, the patient experienced significant fatigue, requiring breaks of 30 minutes to 2 hours to complete tasks like mowing his three-quarter-acre lawn. By the midpoint of his radiation treatment, he reported a significant improvement in energy levels, completing the same task, including trimming, in about one hour without breaks.
Physical Activity: Despite undergoing radiation therapy, the patient maintained a rigorous daily routine, including seven-mile walks, without interruption.
Work and Lifestyle: The patient continued to work full-time throughout the treatment period, with no decline in productivity or energy levels.
Side Effects and Tolerance: The patient reported no common side effects of radiation therapy, such as fatigue, nausea, or skin irritation. Medical staff, including oncologists and nurses, expressed surprise at his resilience and energy, as they had initially predicted a significant decline in his condition.
Sleep Quality: The patient reported consistently sound sleep during the treatment process, which he attributed to the nightly use of the Tesla BioHealer.

### Clinical Outcome:

Upon completing 33 radiation treatments, the patient's oncologist confirmed the therapy's success in treating both the primary prostate cancer and metastatic lesions in the pelvic region. The patient completed the treatment without complications or adverse effects, attributing his enhanced energy and resilience to the continued use of the Tesla BioHealing device

### Example 100. Biophoton Co-Therapy to Enhance Tolerance to Cancer Treatments for Optimized Outcomes—Case 3

A 55-year-old Caucasian male was diagnosed with advanced squamous cell carcinoma of the esophagus. After achieving partial remission through chemotherapy, he was referred for radiotherapy (RT) at the University of Pittsburgh Medical Center (UPMC) Hillman Cancer Center as the next phase of treatment. Within two weeks of RT, the patient experienced escalating side effects, including severe fatigue, nausea, and localized pain in the irradiated area. His physical and psychological well-being were significantly impaired, putting his compliance with the RT protocol at risk. At the time of his participation in a pilot clinical study, the patient had extreme fatigue, could only consume liquid food, and exhibited a marked decline in energy levels.

To support recovery and mitigate the adverse effects of RT, the patient, a medical professional himself, opted to incorporate biophoton co-therapy. Fourteen Tesla BioHealing® Automatic Biophoton Generators (ABGs) were positioned under his bed for extended periods, up to 24 hours daily. The biophoton sessions were administered concurrently with RT, aiming to leverage biophotons' proposed cellular healing and immunomodulatory properties to enhance RT tolerance and reduce the overall cancer burden.

Within the first week of biophoton co-therapy, the patient reported reduced fatigue and an improved sense of energy. He gradually regained his appetite and transitioned to better nutritional intake, which allowed him to endure RT sessions more effectively. Additionally, pain levels decreased, reducing the need for pharmacological interventions. Improvements in overall quality of life, as measured by the SF-36 Score, were documented during the initial four-week study period, as shown in FIG. 47.

Clearly, the patient's life quality was gradually improved during the 4-week study period.

By the end of the 6-month treatment, the imaging studies confirmed complete remission, with no detectable signs of malignancy. A biopsy confirmed a cancer-free status. He then participated in several fishing competition events.

Outcome and Follow-Up: The patient was monitored over the following 12 months with periodic imaging and physical exams. No cancer recurrence was observed, and the patient reported sustained energy levels and absence of the RT-related adverse effects. These findings suggest that biophoton co-therapy aided in the post-RT treatment and recovery phase and potentially contributed to sustained remission.

### Example 101

44 cancer patients voluntarily provided feedback on their experiences with ABGs. Table 6 summarizes the reported improvements among these patients, highlighting the potential benefits of biophoton therapy in addressing cancer-related challenges and enhancing overall quality of life.

TABLE 6
Benefits of Biophoton Co-Therapy Reported by 44 Cancer Patients

| Benefit | Benefit Description | Percentage |
|---|---|---|
| Pain Relief | A significant number of patients reported | 84% |
| | reduced pain, particularly those suffering from | |
| | cancer-related discomfort or chronic | |
| | conditions. | |
| Enhanced Energy Levels | Improved energy and vitality were frequently | 47% |
| | highlighted, suggesting that biophoton co- | |
| | therapy may help patients combat fatigue and | |
| | regain activity levels. | |
| Healing and Recovery | Healing benefits were noted, indicating | 25% |
| | improvements in recovery processes, symptom | |
| | management, and overall health stabilization. | |
| Improved Quality of Life | Positive feedback regarding improved quality | 100% |
| | of life was reflected in testimonials about | |
| | better sleep, reduced stress, and normalization | |
| | of health parameters (e.g., easy to go to work | |
| | and able to help the family). | |
| Symptom Management | Other aspects, such as fatigue alleviation, relief | 60% |
| | from aches and arthritis, and better emotional | |
| | well-being, showing broad applicability in | |
| | supportive care. | |

Biophoton therapy demonstrates promising complementary benefits to traditional cancer treatments by enhancing patients' overall quality of life, alleviating specific symptoms, and supporting physiological recovery. Consistent themes, including significant pain relief, improved energy levels, and accelerated healing, underscore its potential value as a non-invasive and supportive therapy. These findings suggest that biophoton therapy could serve as a co-therapy or mono-therapy to improve treatment tolerance and outcomes in cancer care.

The cases presented in Examples 97-101 highlight the potential of biophoton therapy as a valuable approach in cancer care. The therapy consistently demonstrated benefits, including alleviation of side effects, enhanced energy levels, prevention of pancytopenia, and significant improvements in overall quality of life. These outcomes allowed patients to complete rigorous cancer treatments without interruptions, aligning with emerging evidence that supports the role of biophotons in mitigating the physiological challenges associated with cancer therapies.

The exceptional tolerance observed in patients undergoing chemotherapy, radiotherapy, and targeted therapies raises compelling questions about the mechanisms underlying biophoton therapy's effects.

The current invention provides a bioenergy (biophoton) generator to generate and release the life force to benefit a biological system, like a man, an animal, a plant, and a fermentation system. The bioenergy generator 100 can be manufactured with different bioenergy levels to meet a variety of different needs for life force. The bioenergy was produced and utilized to enable a part, or an entire body of a mammal to increase the life force of cells in the body, to increase stem cells, to promote cell growth, to facilitate cell repair, to enhance cell detoxification, and to reduce cell destruction. Hence, the cells, tissues, organs, systems, and the entire body of a mammal become healthy. The invention is a generator of the life force, which is a three-dimensional (3D) bioenergy field, and a method of using the 3D bioenergy to empower a mammal to restore and maintain the normal body health and function and to heal the body. Also, the simultaneous generation of the bioenergy and far-infrared wave can be made in one device. A mammal can receive the benefits of bioenergy and far-infrared waves at the same time.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

1. A method of preventing or mitigating a side effect of a cancer treatment in a subject, the method comprising:
(a) placing one or more biophoton generators close to the subject within a distance; and
(b) keeping the one or more biophoton generators close to the subject daily over a period of time;
wherein the subject is either scheduled to receive or is receiving the cancer treatment.

2. The method of claim 1, wherein the cancer treatment is radiotherapy, chemotherapy, or targeted therapy.

3. The method of claim 1, wherein the side effect is selected from the group consisting of pancytopenia, fatigue, pain, nausea, vomiting, hair loss, anemia, weight loss, and organ toxicity.

4. The method of claim 1, wherein the method further enhances the adherence of the cancer treatment, improves the efficacy of the cancer treatment, and/or improves the subject's life quality.

5. The method of claim 1, wherein the subject has a solid tumor or blood cancer.

6-7. (canceled)

8. The method of claim 1, wherein the method uses 2 to 20 biophoton generators.

9. The method of claim 1, wherein the biophoton generator is kept close to the subject for 5 minutes to 24 hours per day.

10-11. (canceled)

12. The method of claim 1, wherein the distance in step a) is from 1 cm to 20 meters.

13. The method of claim 1, wherein the period of time in step b) is from one month to 5 years.

14-15. (canceled)

16. A method of treating cancer in a subject in need therefore, the method comprising:
(a) administering a standard cancer treatment to the subject;
(b) placing one or more biophoton generators close to the subject within a distance; and
(c) keeping the one or more biophoton generators close to the subject daily over a period of time.

17. The method of claim 16, wherein the standard cancer treatment is radiotherapy, chemotherapy, or targeted therapy.

18. The method of claim 16, wherein the method reduces the side effect of the standard cancer treatment, such as pancytopenia, fatigue, pain, nausea, vomiting, hair loss, anemia, weight loss, or organ toxicity.

19. The method of claim 16, wherein the method enhances the adherence of the cancer treatment, improves the efficacy of the cancer treatment, and/or improves the subject's life quality.

20. The method of claim 16, wherein the subject has a solid tumor or blood cancer.

21. (canceled)

22. The method of claim 16, wherein the method uses 2 to 20 biophoton generators.

23. The method of claim 16, wherein biophoton generators is kept close to the subject for 5 minutes to 24 hours per day.

24-25. (canceled)

26. The method of claim 16, wherein the distance in step b) is from 1 cm to 20 meters.

27. The method of claim 16, wherein the period of time in step c) is from one month to 5 years.

28. (canceled)

29. A method of treating cancer in a subject in need therefore, the method comprising:
(a) placing one or more biophoton generators close to the subject within a distance; and
(b) keeping the one or more biophoton generators close to the subject daily over a period of time.

30-31. (canceled)

32. The method of claim 29, wherein the subject has a solid tumor or blood cancer.

33-39. (canceled)
